(19) United States
(12) Patent Application Publication
(10) Pub. No.: US 2017/0299530 A1
(43) Pub. Date: Oct. 19, 2017

(54) COMPOUNDS AND METHODS FOR ANALYSIS AND SYNTHESIS OF SACCHARIDE COMPOUNDS, AND METHOD FOR QUANTITATING SACCHARIDE

(71) Applicant: ACADEMIA SINICA, Taipei (TW)

(72) Inventor: Wen-Bin YANG, Taipei (TW)

(73) Assignee: ACADEMIA SINICA, Taipei (TW)

(21) Appl. No.: 15/640,757

(22) Filed: Jul. 3, 2017

Related U.S. Application Data
(63) Continuation-in-part of application No. 14/569,368, filed on Dec. 12, 2014.
(60) Provisional application No. 61/915,316, filed on Dec. 12, 2013.

(51) Int. Cl. G01N 24/08; G01N 33/02; G01N 33/14; G01N 33/50

(52) U.S. Cl. CPC G01N 24/087; G01N 33/50; G01N 33/025; G01N 33/143; G01N 2458/15

(57) ABSTRACT

Provided is a method for quantitating a saccharide in a liquid sample. The method comprises incubating the liquid sample with 2,3-naphthalenediamine in the presence of iodine to allow a naphthimidazole group to be linked to the saccharide to obtain a first mixture; obtaining an ¹H-NMR spectrum of the first mixture; and comparing, in said ¹H-NMR spectrum, the intensity or integral of a proton signal corresponding to the saccharide to the intensity or integral of a proton signal corresponding to an internal standard present in the first mixture.

### CROSS REFERENCE TO RELATED APPLICATIONS

This application is a continuation-in-part of copending application Ser. No. 14/569,368, filed on Dec. 12, 2014, which claims priority under 35 U.S.C. §119(e) to U.S. Provisional Application No. 61/915,316, filed on Dec. 12, 2013, all of which are hereby expressly incorporated by reference into the present application.

### FIELD OF THE INVENTION

The present invention relates to new compounds and method for analysis and synthesis of saccharide compounds. The present invention also relates to a method for quantitating a saccharide in a liquid sample by using NMR.

### BACKGROUND OF THE INVENTION

Saccharides are the one of most abounding materials in the world. Saccharides are presented in various compositions and of structures at natural materials such as foods, herbs and drugs. In addition, saccharides play essential biological functions in glycocojugates, for instance, O-, N-glycans in glycoproteins, O/M-antigens in lipopolysaccharide (LPS). Therefore, it is important to determine what saccharides a natural material is composed, and which carbohydrate structure the saccharides are of. However, most saccharides lack charge and UV absorbing, and they are difficult to be separated and detected by conventional photometric (e.g. UV or fluorescence in liquid chromatography) and electric charge (e.g. CE or HPAEC-PAD) methods.

Chemical structure analysis of saccharides is still highly challenged because the primary structural analysis of glycans (saccardises) or glycoproteins remains challenging due to variable composition, linkage, branching and anomericity of the constituent mono-saccharides in combination with the general heterogeneity. One traditional method is to detect methylacetylalditols by GC-MS to obtain the linkages information of oligo-/poly-saccharidises. The other method is to cleavage per-methylated oligo-/poly-glycan by tandem mass spectrometry (MS-MS) to obtain the linkages information.

Various analytical strategies to analyze the composition and primary structure of biological saccharides were provided but provided poor results. Recently, one solution was provided to develop a series of detection methods for native and derivatized ('tagged’) glycans. An analytical platform for a quantitative and qualitative measure of glycan sequencing was reported, wherein to confirm the structures, the sequence of a particular glycan (with/without 2AB tagged) is determined by sequential exoglycosidases digestion. Alternatively, a method of non-enzymatic analysis, such as NMR, MS, LC, GC, was provided to determine the structure of the glycan. However, it is difficult to separate between D and L configurations of sugars, and identify which configuration of a sugar is, D or L configuration because D or L configurations are optical isomers of each other, or an enantiomeric pair.

Carbohydrates are found in daily foods. Glycans are the polymer forms of sugar, such as starch, amylopectin, cellulose and fiber that exist in crop foods. It is important to understand the sugar ingredients in crop foods. Carbohydrates are also used as “added sugar” in soft drinks, cookies, candies and foods. For example, the added sugar in beverage can be sucrose, high-fructose corn syrup (HFCS) and other sweeteners. Differential sugar profiling plays an essential role in energy intake. Though carbohydrates are needed for health, excessive uptake of sugar may induce obesity, decayed teeth, and chronic diseases. For this reason, foods of low glycemic index (GI) are suggested for diabetes patients. Furthermore, many countries have introduced the sugar tax and soft-drink tax in order to reduce sugar consumption. According to the scientific recommendation by World Health Organization (WHO), the appropriate sugar intake is 25 grams per day. Since August 2015, Taiwan Food & Drug Administration (TFDA) has proposed to regulate common sugars in foods, including glucose (Glc), galactose (Gal), fructose (Fru), lactose (Lac), maltose (Mal) and sucrose (Suc). The amounts of sugars must be labeled in the “Nutrition Facts Panel” for the products of beverage and food. Even though the information of sugar content surely benefits consumers, this regulation will pose challenges to the food industry because it is difficult to quantify the sugar contents in beverages and there is no efficient analytic method for determination of the glycans in food crops.

Carbohydrate molecules lack responsive chromophores, thus analyses of carbohydrates are often performed by labeling with appropriate reagents, such as 2-aminobenzamide (2-AB), 2-aminopyridine (2-AP), phenylhydrazine, 1-phenyl-3-methyl-5-pyrazolone (PMP) and 2,3-naphthalenediamine, to form the derivatives for UV-vis or fluorescence detection. Such derivatization of carbohydrates may also increase hydrophobicity to improve ionization for mass spectrometric analysis (Lin et a/., J. Org. Chem. 2008, 73, 3848-3853; and Lin et al., Rapid Commun. Mass Spectrom. 2010, 24, 85-94). Except for high-performance anion-exchange chromatography with pulsed amperometric detection (HPAEC-PAD) that can be used to analyze original carbohydrates, prior introduction of chromophore/fluorophore to carbohydrates are usually required for detection using chromatography and electrophoresis. Labeling aldoses with 2,3-naphthalenediamine via an iodine-promoted oxidative condensation reaction to form the naphthimidazole (NAIM) derivatives is a highly sensitive method for UV-vis, fluorescence and mass analyses. For example, the sugar composition in beverages and dietary foods can be determined by HPLC analysis via their NAIM derivatives. According, there is still a need for a more time-effective method other than a method relying on HPLC analysis.

So far, although there are some methods have been used in saccharide labeling for characterization or identification of a saccharide, there are still having some limitations. Therefore, to develop a cost-effective and sensitive method for carbohydrate analysis is desired.

### SUMMARY OF THE INVENTION

The present invention is to provide a new approach for glycan sequencing of saccharides, including new isotope-labelled compounds that can be used as agents and the method using the new isotope-labelled compounds.

In one aspect, the present invention provides a compound library comprising benzimidazole derivated saccharides, having a general formula I, S-BIM, wherein S is a sugar moiety and BIM is a benzimidazole.

In some embodiment of the invention, the sugar moiety is a saccharide such as an aldose, a ketoacid sugar or a ketosugar.

In another aspect, the present invention provides benzimidazole-like derivated compounds having a general formula II, S-Y-W, wherein S is a sugar moiety, W is a benzimidazole like moiety, and Y is a function providing moiety.

In some embodiment of the invention, the function providing moiety may be an isotope, a halogen, a peptide, a protein, a biotin, a dye, a fluorescein isothiocyanate (FITC), or a solid support such as a resin, a (nano) particle, a plate or a chip.

In some embodiment of the invention, the benzimidazole like moiety is a moiety derivated from imidazole, such as benzimidazole, quinoxalinone or hydrazine.

According to the invention, some examples of benzimidazole-like derivated compounds include:
(1) saccharide-Y-benzimidazoles (also called as “the SYBIM derivatives”) having a general formula II(a), SYBIM, wherein S is a saccharide (such as an aldose), Y is a function providing moiety (such as an isotope) and BIM is benzimidazole;
(2) ketoacid-Y-quinoxalinones (also called as “the SYBQX derivatives”) having a general formula II(b), SYBQX, wherein S is a ketoacid sugar, Y is a function providing moiety (such as an isotope) and BQX is quinoxalinone; and
(3) ketosugar-Y-hydrazine (also called as “the SYBHZ derivatives”) having a general formula II(c), SYBHZ, wherein S is a ketosugar, Y is a function providing moiety (such as an isotope), and BHZ is hydrazine.

In a further aspect, the invention provides new isotope-labelled compounds having a general formula II(a)′, S-Y′-BIM, wherein S is a sugar moiety, Y′ is an isotope-containing sensor moiety, and BIM is benzimidazole.

In one embodiment of the invention, the isotope-containing sensor moiety is an isotope, such as an isotope of hydrogen or halogen.

In one example of the invention, the isotope is selected from the group consisting of ¹H, ²H, ³H, ¹⁹F, ³⁵Cl, ³⁷Cl, ⁷⁹Br, and ⁸¹Br. In one particular example of the invention, the isotope is ¹⁹F.

In some examples of the invention, the isotope is selected from the group consisting of ¹H, ²H, ³H, ⁹B, ¹⁰B, ¹¹B, ¹³C, ¹⁴N, ¹⁵N, ¹⁶O, ¹⁸O, ¹⁹F, ³¹P, ³³S, ³⁵Cl, ³⁷Cl, ⁷⁹Br, and ⁸¹Br.

Accordingly, the invention provides particular isotope labelled compounds for saccharide analysis, for example, D-Gal-BIM/L-Gal-BIM (Gal=galactose) and D-Fuc-BIM/L-Fuc-BIM (Fuc=fucose) that are labelled with an isotope, such as ¹⁹F.

According to the invention, the new isotope-labelled compounds can be used as agents for saccharide analysis, by such as nuclear magnetic resonance spectroscopy (NMR), liquid chromatography (LC), gas chromatography (GC), high-pressure liquid chromatography (HPLC) or mass spectrometry (MS).

In addition to the use as agents for saccharide analysis, the isotope labelled compounds can be used for diagnosis or prognosis of saccharides, and also can serve as physiological probes or cell-function reporters.

In some embodiments of the invention, the sugar moiety is an aldose, a ketoacid sugar or a ketosugar.

In one example of the invention, Y is a UV or a fluorescent. Accordingly, the SYBIM can be used as a tool to facilitate the glycans separation or structural identification by using enzymatic degradation. Analysis for the sugar types, linkages or L-/D-forms of a glycan can be obtained.

In a yet further aspect, the present invention provides a method for preparing a neoglycopeptide or neoglycoprotein containing benzimidazole, comprising allowing an amino acid building block to be linked to a DAB linker having the structure:

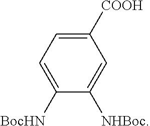 [SMILES: CC(C)(C)OC(=O)Nc1ccc(C(=O)O)cc1NC(=O)OC(C)(C)C]

to obtain DAB-peptides by solid phase synthesizer.

In a still further aspect, the present invention provides a method for saccharide analysis by using the isotope labelled compounds detected by an appropriate measurement such as nuclear magnetic resonance spectroscopy (NMR) liquid chromatography (LC), gas chromatography (GC), high-pressure liquid chromatography (HPLC) or mass spectrometry (MS). The method may be used for characterization of a glucan-containing substance or mixture, including for determining D/L form of a sugar, components of a sugar mixture, structure of a glycan or a glycopeptide, nature of a glycosidic linkage, and glycan sequence of an unknown sugar, glycopeptide or other glycoconjugates.

In one embodiment of the invention, the structures of oligo-/poly-saccharides could be well analyzed by the SYBIM derivatives by a spectrometry. According to the invention, the spectrometry is selected form the group consisting of IR, NMR, MS, LC, GC, HPLC and any combination thereof. In one example of the invention, the isotope labelled compound can be used and analysized by IR, NMR, MS, LC, GC, HPLC, Raman or an enzymatic method to identify alpha-/beta-anomeric center at C-1 position, stereoisomers of a saccharide or D-/L-configuration.

In one example of the invention, the method provides a rapid identification of N-/O-glycans and other type glycans.

In a further aspect, the present invention provides a method for enzymatic analysis of glycosidase activity or its inhibitors by using one or more SYBIMs as substrates.

In one example of the invention, the method for testing activity of a glycosidase enzyme, comprises the steps of:
(a) contacting a sample of the glycosidase enzyme with the compound as defined in any one of claims 8 and 10-24 to form a SYBIM derivated compound; and
(b) determining the glycosidase activity of the SYBIM derivated compound.

On the other hand, the method for screening a glycosidase inhibitor, comprises the steps of:
(a) contacting a camdodate with the compound as defined in any one of claims 8 and 10-24 to form a SYBIM derivated compound; and
(b) determining the activity in inhibition to glycosidase of the SYBIM derivated compound in addition with a glycosidase emzyme.

In a yet further aspect, the present invention provides a simple method for preparing the SYBIM derivatives with various functions as desired.

In a still further aspect, the invention provides method for glycan sequencing by stepwise chemical degradation of SYBIMs formed by using the compounds as defined in any one of the previous claims, which is used for structural identification of a glycan. Also, an automatic glycan synthesizer or sequencer for performing the method of the invention.

In a further aspect, the present invention features a method for determining the sequence of a glycan(N) comprising N monosaccharide subunits, comprising the following steps: (i) attaching a first benzimidazole-like compound to a reducing end of the glycan(N) to obtain a modified glycan(N); (ii) subjecting the modified glycan(N) to a hydrolysis reaction to obtain a first monosacharide modified by the first benzimidazole-like compound, and a glycan(N-1) comprisng N-1 monosaccharide subunits; (iii) attaching a second benzimidazole-like compound different from the first benzimidazole-like compound to a reducing end of the glycan(N-1) to obtain a modified glycan(N-1); and (iv) subjecting the modified glycan(N-1) to a hydrolysis reaction to obtain a second monosacharide modified by the second benzimidazole-like compound, and a glycan(N-2) comprising N-2 monosaccharide subunits.

In certain embodiments of the present invention, each of the first and second benzimidazole-like compound is a compound selected from the group consisting of 2,3-naphthalenediamine, 4-fluorophenyl diamine, 4,5-difluorophenyl diamine, 4-trifluoromethanephenyl diamine, 4-chlorophenyl diamine, 4,5-dichlorophenyl diamine, 4-bromophenyl diamine, 4,5-dibromorophenyl diamine, ortho-phenyl diamine, 1,2-phenyl diamine, and 4-carboxylphenyl diamine. Preferably, the compound is isotope-labelled.

According to the present invention, the identity of the first and second monosacharides can be determined by NMR, HPLC, or LC/MS.

In a still further aspect, the invention provides a method method for quantitating a saccharide in a liquid sample, comprising incubating the liquid sample with 2,3-naphthalenediamine in the presence of iodine to allow a naphthimidazole group to be linked to the saccharide to obtain a first mixture; obtaining an ¹H-NMR spectrum of the first mixture; and comparing, in said ¹H-NMR spectrum, the intensity or integral of a first proton signal corresponding to the saccharide to the intensity or integral of a second proton signal corresponding to an internal standard present in the first mixture.

The internal standard may be selected from the group consisting of DMSO,

[symbol]

In certain embodiments of the present invention, said liquid sample is prepared by acid hydrolysis of a solid sample.

According to the present invention, the first proton signal is a characterizing proton signal of the saccharide, and the second proton signal is a characterizing proton signal of the internal standard.

The saccharide to be quantified includes but is not limited to glucose (Glc), galactose (Gal), fructose (Fru), lactose (Lac), maltose (Mal), sucrose (Suc), mannose (Man), rhamnose (Rha), arabinose (Ara), glucuronic acid (GlcUA), and N-acetylglucose (GlcNAc).

According to one preferred embodiment of the present invention, the first proton signal is a vinyl proton signal.

The internal standard includes but is not limited to DMSO, tetramethylsilane, and (CH₃)₃SiCO₂Na.

In certain embodiments of the present invention, the internal standard is DMSO. Preferably, the second proton signal includes the NMR signals of six protons of the two methyl groups of DMSO at δ 2.73.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiment which is presently preferred. It should be understood, however, that the invention is not limited to this embodiment.

In the drawings:

FIG. 1 shows a scheme of the preparation of neoglycopeptides or neoglycoproteins containing benzimidazole as a linker by using DAB.

FIG. 2 shows a scheme of the glycan sequencing method for determining the glycan structure according to the invention.

FIG. 3 shows ¹H-NMR spectra (600 MHz) in D₂O solution containing 0.1% (CH₃)₂SO: (A) Glc, (B) Glc-NAIM, (C) Gal-NAIM, (D) Mal-NAIM and (E) Lac-NAIM. The aromatic protons of NAIM derivatives in the range of δ 7.2-8.2 are not shown for clearance. The signal of HDO is set at δ 4.80, and the signal of internal standard (CH₃)₂SO occurs at δ 2.73.

FIG. 4 shows ¹ H-NMR spectra (600 MHz) in D₂O containing 0.1% (CH₃)₂SO: (A) a mixture of 4 aldoses (Glc, Gal, Mal and Lac, 5 mg of each sugar). The mixtures of NAIM derivatives were prepared from the corresponding aldose mixtures, containing each aldose in 5 mg (B), 2.5 mg (C), 1.25 mg (D) and 0.25 mg (E), respectively. The aromatic protons of NAIM derivatives in the range of δ 7.2-8.2 are not shown for clearance. The signal of HDO is set at δ 4.80, and the signal of internal standard (CH₃)₂SO occurs at δ 2.73.

FIG. 5 shows ¹H-NMR spectra (600 MHz) in D₂O containing 0.1% (CH₃)₂SO: (A) a mixture of 6 sugars (Glc, Gal, Fru, Mal, Lac and Suc, 5 mg of each sugar), and (B) four aldoses are labeled as Glc-NAIM, Gal-NAIM, Mal-NAIM and Lac-NAIM, along with partially conversion of Fru to Fru-enamine [A] and α-amino aldehyde [B], and Suc retains without modification. The aromatic protons of NAIM derivatives in the range of δ 7.2-8.2 are not shown for clearance. The signal of HDO is set at δ 4.80, and the signal of internal standard (CH₃)₂SO occurs at δ 2.73.

FIG. 6 shows calibration lines of (A) Glc, (B) Gal, (C) Mal, (D) Lac, (E) Suc and (F) Fru: x is the relative integration of the selected proton, proportional to the 6 protons of (CH₃)₂SO at 0.033% concentration (4.3 μmol), in the ¹H-NMR spectrum; and y is the weight (in mg) of the parental sugar.

FIG. 7 shows ¹H-NMR spectra (600 MHz) in D₂O containing 0.1% (CH₃)₂SO: (A) fructose, and (B) fructose treated with 2,3-naphthalenediamine. Inset: aromatic protons in the range of δ 7.4-8.2 and a singlet at δ 9.24. The signal of HDO is set at δ 4.80, and the signal of internal standard (CH₃)₂SO occurs at δ 2.73.

### DETAILED DESCRIPTION OF THE INVENTION

Unless defined otherwise, all technical and scientific terms used herein have the same meaning as commonly understood by a person skilled in the art to which this invention belongs.

The term “isotope” as used herein, also known as “isotopic marker” or “isotopic label,” refers to one or more variants of a particular chemical element, while all isotopes of a given element have the same number of protons in each atom, they differ in neutron number. Different isotopes of a single element occupy the same position on the periodic table. Each isotope of a given element has a different mass number. The isotopes are commonly used in chemistry and/or biochemistry to learn chemical reactions and interactions, which are stable and can be detected separately from the other atoms of the same element. Examples of the isotope include ¹HH, ²H, ³H, ⁹B, ¹⁰B, ¹¹B, ¹²C, ¹³C, ¹⁴C, ¹⁴N, ¹⁵N, ¹⁶O, ¹⁸O, ¹⁹F, ³¹P, ³³S, ³⁵Cl, ³⁷Cl, ⁷⁹Br, and ⁸¹Br. In one embodiment of the invention, the isotope is a halogen. In a particular example of the invention, the isotope is ¹⁹F.

The term “benzimidazole” as used herein, refers to a heterocyclic aromatic organic compound, consisting of the fusion of benzene and imidazole, which is of the chemical structure below:

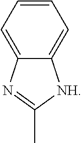

The term “saccharide”, also known as “sugar,” “glycan” or “carbonydrate,” as used herein, refers to a molecule consisting only of carbon (C), hydrogen (H), and oxygen (O), usually with an empirical formula Cₘ(H₂O)ₙ (where m could be different from n), including monosaccharides, disaccharides, oligosaccharides, and polysaccharides.

The term “aldose” as used herein refers to a monosaccharide that contains only one aldehyde group (—CH═O) per molecule, which has a general formula of Cₙ(H₂O). Because they have at least one asymmetric carbon center, aldoses with three or more carbon atoms exhibit stereoisomerism, and accordingly an aldose may exist in either a D form or L form of a Fischer projection. Examples of aldose include but are not limited to a diose such as glycolaldehyde; a triose such as glyceraldehyde; a tetrose such as erythrose or threose; a pentose such as ribose, arabinose, xylose or lyxose; a hexose such as allose, altrose, glucose, mannose, fucose, gulose, idose, galactose or talose. Particular examples in the present invention are glucose, fucose, xylose, mannose and galactose.

The term “ketosugar” as used herein refers to any of various carbohydrates containing a ketone group. Examples of a ketosugar include but are not limited to dihydroxyacetone, tetroses: erythrulose, pentoses: ribulose, xylulose, fructose, psicose, sorbose, tagatose, sedoheptulose, etc. Particular examples in the present invention are fructose and sorbose.

The term “ketoacid” as used herein refers to an organic compound containing a carboxylic acid group and a ketone group. Examples of ketoacidsugar include but are not limited to alpha-keto acids or 2-oxoacids, having a keto group adjacent to a carboxylic acid, such as pyruvic acid; beta-keto acids or 3-oxoacids, having a ketone group at the second carbon from a carboxylic acid, such as acetoacetic acid; and gamma-keto acids or 4-oxoacids, having a ketone group at the third carbon from a carboxylic acid, such as levulinic acid. Particular examples in the invention are sialic acid, Neu-5Gc (N-glycolylneuraminic acid), KDN (2-keto-3-deoxy-D-glycero-D-galacto-nononic acid) and KDO (3-Deoxy-D-manno-oct-2-ulosonic acid).

In the invention, a compound library comprising benzimidazole derivated saccharides is constructed. The benzimidazole derivated saccharides have a general formula I, S-BIM, wherein S is a sugar moiety and BIM is a benzimidazole. The sugar moiety may be a saccharide (such as aldose), a ketosugar or a ketoacid.

According to the invention, the compound library can be contracted by all or parts of the compounds as provided.

The present invention also provides benzimidazole-like derivated compounds having a general formula II, S-Y-W, wherein S is a sugar moiety, Y is a function providing moiety, and W is a benzimidazole like moiety.

In the invention, the function providing moiety may be an isotope, a halogen, a peptide, a protein, a biotin, a dye, a fluorescein isothiocyanate (FITC), or a solid support such as a resin, a (nano) particle, a plate or a chip. The benzimidazole like moiety, which is a moiety derivated from imidazole, such as benzimidazole, quinoxalinone or hydrazine.

The benzimidazole-like derivated compounds include:
(1) saccharide-Y-benzimidazoles (also called as “the SYBIM derivatives”) having a general formula II(a), SYBIM, wherein S is a saccharide (such as an aldose), Y is a function providing moiety (such as an isotope) and BIM is benzimidazole;
(2) ketoacid-Y-quinoxalinones (also called as “the SYBQX derivatives”) having a general formula II(b), SYBQX, wherein S is a ketoacid sugar, Y is a function providing moiety (such as an isotope) and BQX is quinoxalinone; and
(3) ketosugar-Y-hydrazine (also called as “the SYBHZ derivatives”) having a general formula II(c), SYBHZ, wherein S is a ketosugar, Y is a function providing moiety (such as an isotope), and BHZ is hydrazine.

In a particular aspect, the invention provides new isotope-labelled compounds having a general formula IIa′, S-Y′-BIM, wherein S is a sugar moiety, Y′ is an isotope and BIM is benzimidazole.

In one embodiment of the invention, the isotope is a halogen. The isotope is selected from the group consisting of ¹H, ²H, ³H, 9B, ¹⁰B, ¹¹B, ¹³C, ¹⁴N, ¹⁵N, ¹⁹F, ³¹P, ³³S, ³⁵Cl, ³⁷Cl, ⁷⁹Br, and ⁸¹Br. Preferable examples are ¹H, ⁹Be, ¹⁰B, ¹¹B, ¹⁴N, ³¹P, ³⁵Cl, ³⁷Cl, ⁷⁹Br, and ⁸¹Br. In a particular example of the invention, the isotope is ¹⁹F.

In the invention, the sugar moiety is an aldose, a ketosugar or a ketoacid, including common mono-/oligo-/poly-saccharides, like xylose, ribose, rhamnose, arabinose, fucose, glucose, mannose, galactose, N-acetyl-glucosamine, N-acetyl-galactosamine, glucosamine, galactosamine, glucuronic acid, galacturonic acid, N-acetylneuraminic acid, Neu5Gc, KDO, KDN, fructose, sorbose, and etc. in reducing end of sugar.

In some examples of the invention, D-Gal-BIM/L-Gal-BIM (Gal=galactose) or D-Fuc-BIM/L-Fuc-BIM (Fuc=fucose) that are labelled with an isotope such as ¹⁹F, are provided.

In the invention, the isotope labelled compounds can be used as standard compounds for saccharide analysis, by using such as nuclear magnetic resonance spectroscopy (NMR), liquid chromatography (LC), gas chromatography (GC), high-pressure liquid chromatography (HPLC) or mass spectrometry (MS). The compounds can be used for analysis of the composition, components, structure, D/L-configuration of a saccharide. Because benzimidazole ring has paramagnetic atom(s), these compounds show significant separation signals with chemical shifts and integration, which can be measured by NMR, HPLC, MS for sugar qualification and quantification.

According to the invention, the isotope labelled compounds are first synthesized to provide standard compounds (including SYBIMs, SYBQXs and. SYBHZs, wherein Y is an isotope) by an appropriate measurement such as NMR, LC, GC, HPLC or MS measurement. The lowest level to be detected in the methods using these compounds for saccharide analysis is 10⁻⁶˜10⁻³ mole by NMR and in 10⁻⁹˜10⁻¹⁵ mole by LC and MS measurement.

According to the invention, these compounds may be synthesized in one-pot by using benzimidazole as a linker between the sugar moiety and the function providing moiety, such as the method disclosed in Lin et al. (“Using Molecular Iodine in Direct Oxidative Condensation of Aldoses with Diamines: an Improved Synthesis of Aldo-benzimidazoles and Aldo-baogtunudazikes for Carbohydrate Analysis”, J. Org. Chem. 73: 3848-3853, 2008), which is incorporated herein by reference in its entirety. According to the invention, these compounds may be used for any purposes.

In the invention, the novel SYBIM derivatives can be prepared by Y-phenyldiamine and Y-phenylhydrazine according to Scheme 1

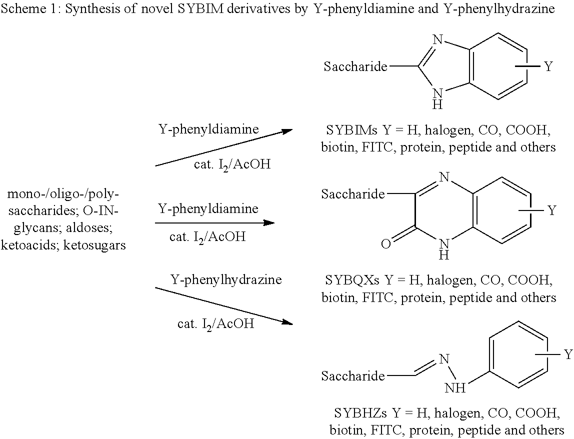

In certain examples of the present invention, various chemical shifts of sugar-FBIMs derivatives (i.e., SYBIMs wherein Y is ¹⁹F) in ¹⁹F-NMR were found (data not shown). The mixtures of various sugar-FBIMs can be analysized by ¹⁹F-NMR, and the results (not shown) indicating 11 separated peaks (representing Gal, GalNHAc, GalA, Fuc, Glc, GlcA, Man, Xyl, Rib, Rhamn, Ara, Sia and KDO respectively) by ¹⁹F-NMR when nine kinds of sugar-5FBIMs (˜120 ppm) and two sugar-6FBQXs (˜110 ppm) were randomly mixed. Therefore, these sugar-FBIMs can be used as standard compounds for saccharide identification and quantification.

SYBIMs can be used as standard agents for glycan analysis. Due to the different polarity of sugar-5FBIMs, 5FBIM scarring strong UV absorption at 280 nm could be separated by LC to facilitate sugar separation and identification (data not shown). In addition, the sugar-5,6F₂BIMs could also be separated by LC to facilitate sugar separation and identification (data not shown). The lowest level of SYBIMs to be detected is 10⁻⁶˜10⁻³ mole by NMR, and 10⁻⁹˜10⁻¹⁵ mole by LC and MS measurement (data not shown). Enantiomeric pair of D-sugar-FBIM/L-sugar-FBIM with chiral shift reagent (Europium tri[3-(trifluoromethylhydroxy-methylene)-(+)-camphorate) was identified by ¹⁹F-NMR could be separated.

According to the invention, the structures of oligo-/poly-saccharides could be well analyzed by the SYBIM derivatives using a pectrometry such as NMR, MS, LC, GC and/or HPLC. In some particular examples of the invention, the spectrometry is MALDI-MS, ESI-MS, GC/MS, MS/MS, CE, HPLC, FPLC, IR, Ramon or any other suitable technique. Per-methylated oligo-/poly-glycan-BIMs (e.g. pSYBIMs) were analysized for tandem mass spectrometry (MS-MS) and GC-MS to obtain the linkages information. The overall glycan structures could not be found in detail by using the two traditional methods. However in the invention, for example, alpha-/beta-anomeric center at C-1 position, stereoisomers of saccharides and D-/L-configuration could be analysized in the invention, using the isotope labelled compounds in combination of IR NMR, MS, LC, GC, IR, Raman or enzymatic methods. Similarly, UV/fluorescent labeled SYBIMs should be a useful tool to facilitate the glycans separation and structural identification by using enzymatic degradation to analyze the sugar types, linkages and L-/D-forms. For example, maltohexose-BBIM (M6-NAIM) can be synthesized and used as a substrate for structural identification of a saccharide. The other glycan labeling reagents such as 2AA, 2AB and 2AP should also be used. For instance, the various aldoses are labeled with labeling reagent (2AB) at sugar reducing end by reductive amination with NaBH₃CN as a dye (see Scheme 2). These 2AB (also called “dye” in Scheme 2) labeled glycans and 2AB glycan labeling kits can be purchased and be used for saccharide analysis and structural determination at the same time SYBIM labeled glycans such as a 2AB labeled glycan can be used for glycan sequencing by enzymatic approach.

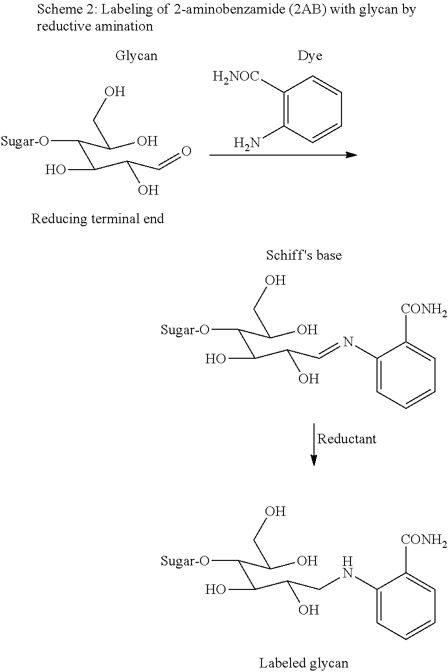

In the present invention, the SYBIM is a key intermedia with simply (in one-step method), safe (with no reductant need, as compared with a toxic reductant, NaBH₃CN, by reductive amination), environmental friendly (with no salts formation, as compared with salts remained in reductive amination reaction) and straightforward (using SYBIMs directly without a desalt or pre-column treatment to avoid the loss of samples).

In one example of the invention, the conversion of fetuin glycans to naphthimidazole (NAIM) derivatives is established by the iodine-promoted oxidative condensation of glycan with 2,3-naphthalenediamine for N-glycan identification by linear ion trap-Fourier transform mass spectrometer (LTQ-FTMS) and liquid chromatography. NAIM derivatization is particularly effective in improving the detection of sialyated glycans. No cleavage of the glycosidic bond occurred under such mild reaction conditions. In MS measurement, an increase of signal intensity was obtained by sialyated-N-glycan-BBIMs (or called sialyated-N-glycan-NAIMs), and improved S/N ratio was also achieved for NAIM derivated N-glycans.

Accordingly, the present invention provides a valuable tool for low abundance glycan identification in complex samples by SYBIM derivatives using NMR, MS, LC, GC, IR, Raman, etc. for structural analysis of saccharides. This invention can also be used to facilitate the characterization and analysis of novel glycans by using SYBIMs in combination of a LC/MS/NMR analysis. In one example of the invention, a rapid method for identification of N-/O-glycans and other type glycans is provided.

The present invention provides a method for enzymatic analysis of glycosidase activity or its inhibitors by using SYBIMs as substrates. In one example of the invention, the oligo-/poly-glycans can be labeled as SYBIM derivatives for its structural analysis by enzymatic assays. In one example of the invention, various linkages of oligosaccharideBBIMs (maltohexoseBBIM, larminarihexoseBBIM, cellohexoseBBIM; 1 mg/each) were prepared. These glycanBBlMs were degraded by special enzymes to learn the real structures of glycans, for example, α-amylase, endo-β-1,3-glucanase and cellulase, respectively. A peak at −116.88 ppm representing the maltohexo-5FBIM was observed in ¹⁹F-NMR spectrum (corresponding to the peak at 4.2 min found in LC; data not shown). Accordingly, the SYBIMs can be used as substrates for enzymatic analysis of glycosidase activity or its inhibitors in combination of an enzyme activity assay or inhibition ability assay, which may be used for drug screening system, wherein the SYBIMs may be replaced for P-nitrophenyl-β-D-glucopyranoside as a substrate. Therefore, the SYBIMs provide an alternative method for activity assay.

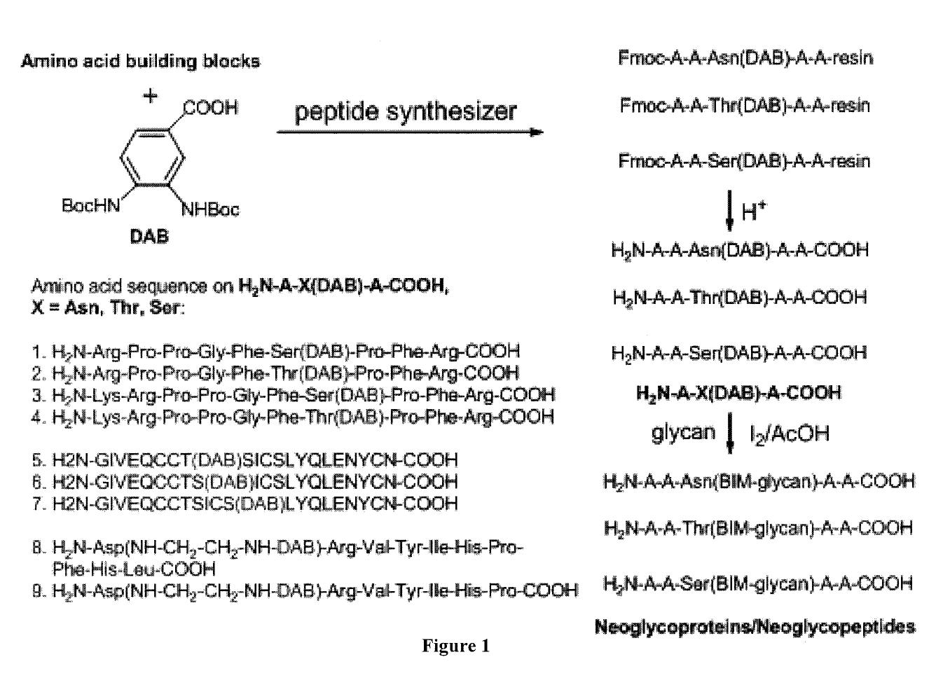

Referring to FIG. 1 showing a scheme for preparation of a neoglycopeptide or neoglycoprotein containing benzimidazole, a DAB having the structure below is used to be linked to an amino acid building block:

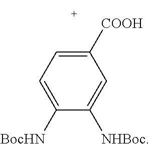 [SMILES: CC(C)(C)OC(=O)Nc1ccc(C(=O)O)cc1NC(=O)OC(C)(C)C]

In one example of the invention, DAB-peptides were obtained by solid phase synthesizer. The DAB linker was set at Asn (N-glycoprotein) or Thr/Ser (O-glycoprotein). Neoglycopeptides/neoglycoproteins, such as N-Glycan-peptide-BIMs and O-Glycan-peptide-BIMs, can be formed by glycans and DAB-peptides. The resulting solution was precipitated and centrifuged to obtain the products, which can be lyophilized to give the pellets of N-Glycan-peptide-BIMs and O-Glycan-peptide-BIMs.

In the invention, the SYBIM derivatives, including glycopeptides or glycoproteins, can be linked to one or more functional groups (e.g. a peptide, a protein, a biotin, a FITC, a dye, a halogen and etc.) and other solid support (such resin, nano particle, plate and chip) to enrich the release or interaction with a protein of the glycan. In some examples of the invention, the glycosylation sites of glycoproteins include Asn; Lys; Arg (N-Type); Thr; Ser; Tyr (O-Type); Cys (S-Type) and Asp; Glu (E-Type). Accordingly, the present invention provides a simple method for preparing the SYBIM derivatives with various functions as desired.

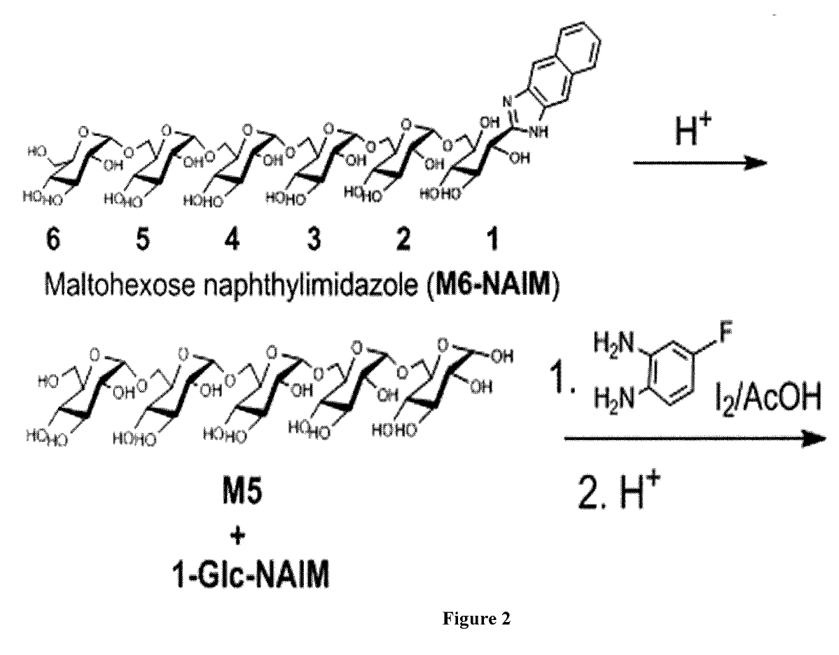

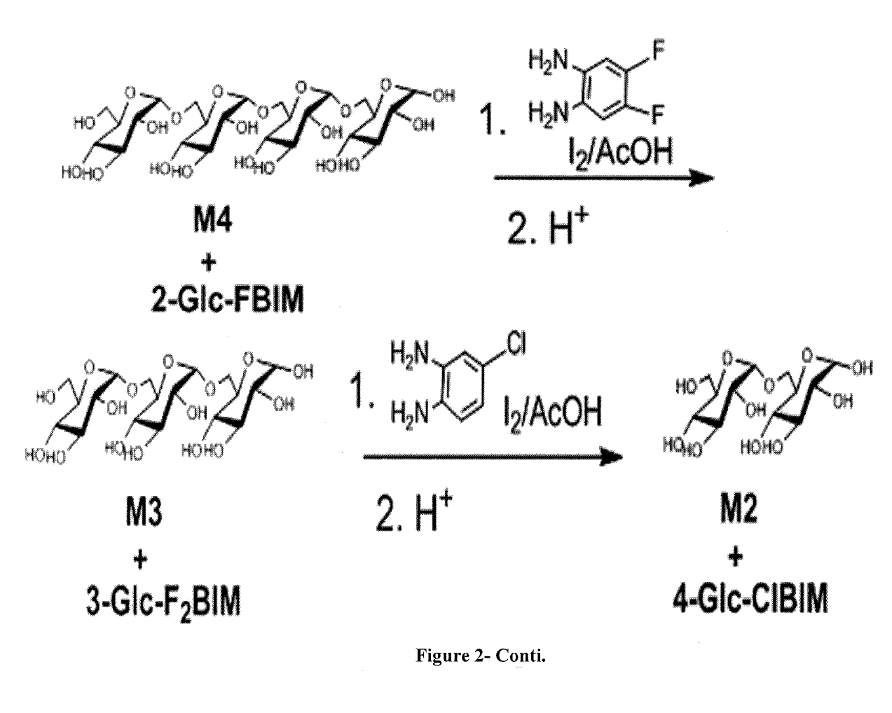

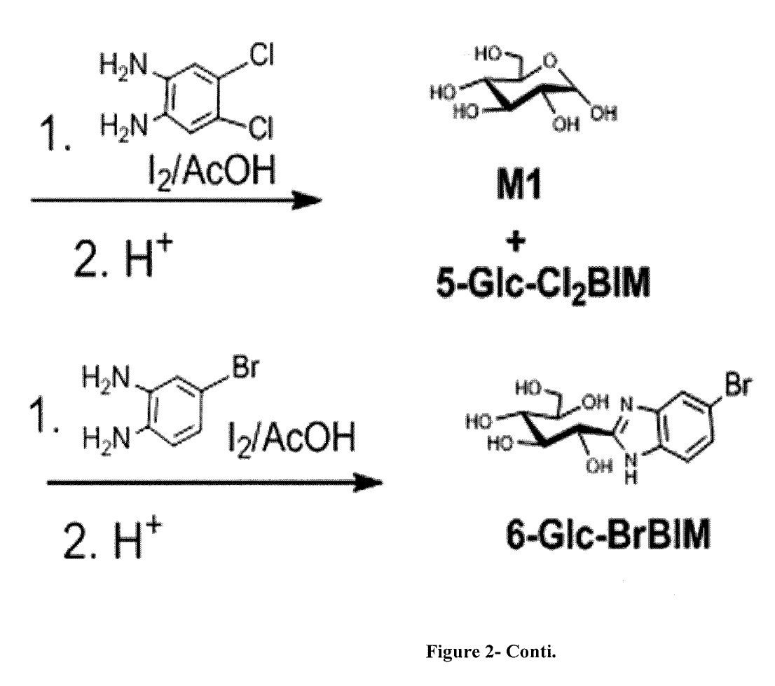

Furthermore, the invention provides a method for glycan sequencing by stepwise chemical degradation of SYBIM. More particularly, the method comprises the following steps: (i) attaching a first benzimidazole-like compound to a reducing end of the glycan(N) to obtain a modified glycan(N); (ii) subjecting the modified glycan(N) to a hydrolysis reaction to obtain a first monosacharide modified by the first benzimidazole-like compound, and a glycan(N-1) comprisng N-1 monosaccharide subunits; (iii) attaching a second benzimidazole-like compound different from the first benzimidazole-like compound to a reducing end of the glycan(N-1) to obtain a modified glycan(N-1); and (iv) subjecting the modified glycan(N-1) to a hydrolysis reaction to obtain a second monosacharide modified by the second benzimidazole-like compound, and a glycan(N-2) comprising N-2 monosaccharide subunits. One embodiment of the invention is illustrated in FIG. 2, which is established for sugar structural analysis. Accordingly, the invention also provides an automatic glycan synthesizer or sequencer based on the method of the invention.

The present invention is described more specifically with reference to the following examples. The following examples are given for the purpose of illustration only and are not intended to limit the scope of the invention.

### EXAMPLES

Instrumentation

The MALDI-TOFMS used to acquire the spectra is an Ultraflex II MALDI-TOF/TOF mass spectrometer (Bruker Daltonik GmbH, Bremen, Germany). Typically, spectra were obtained by accumulating 800-1000 laser shots for quantification. Laser power was fixed in 35% and the pulsed ion extraction was adjusted at 250 ns. The NanoLC-ESI-FTMS experiments were done on a LTQ Orbitrap XL ETD mass spectrometer (Thermo Fisher Scientific, San Jose, Calif.) equipped with a nanoelectrospry ion source (New Objective, Inc.), and accela LC system was used (Thermo Fisher Scientific, San Jose, Calif.). The sample solution was injected (5 μl) at 10 μl/min flow rate on to a self-packed pre-column (150 μm I.D.×30 mm, 5 μm, 200 Å). Chromatographic separation was performed on a self-packed reversed phase C18 nano-column (75 μm I.D.×200 mm, 2.5 μm, 90 Å), using 0.1% formic acid in water as mobile phase A and 0.1% formic acid in 80% acetonitrile as mobile phase B operated at 300 nl/min flow rate with gradient from 10% to 40% of mobile phase B. A full-scan MS condition applied with mass range m/z 320-4000, resolution 60,000 at m/z 400. Electrospray voltage was maintained at 1.8 kV and capillary temperature was set at 200° C. All nanoLC-ESI-FTMS was converted to [M+H]⁺by using Xtract (Thermo Fisher Scientific, San Jose, Calif.) and combine all MS spectra to single spectrum. The LaChrom Elite HPLC (Hitachi, Japan) was used to monitor glycan-NAIMs. Samples were dissolved in a HPLC-grade H₂O. A phosphate buffer (100 mM, pH 5.0) solution with 20% of MeOH was used prior to the purification process of the labeled glycans. Reverse-phase C18 column (4.6×250 mm) with flow rate 0.8 ml/min and UV with the wavelength at 330 nm were used to collect glycan-YBIMs from the reaction mixture. NMR studies, ¹H/¹³C NMR and other 1D and 2D experiments were performed on a Bruker Fourier transform spectrometer (AV-600) equipped with a 5 mm DCI dual cryoprobe. ¹⁹F NMR experiments were performed on a Bruker Fourier transform spectrometer (470 MHz). Spectra were obtained at 298 K with solutions of sugar-FBIMs/FBQXs/FBHZs in D₂O, MeOH-d4, DMSO-d6, acetic acid-d4 and the trifluoritoluene (−63.72 ppm) or trifluoroacetic acid (−76.55 ppm) was added as an internal standard for calibration. Others spectrometers such as GC (Polaris Q), IR and Raman are also useful for the measurement of SYBIMs.

Materials

Ortho-phenyl diamine, 4-fluorophenyl diamine, 4,5-difluorophenyl diamine, 4-trifluoromethane phenyl diamine were purchased from Matrix Scientific (Columbia S.C.). Matrices of 2,5-dihydroxybenzoic acid (2,5-DHB), 4-fluorophenylhydrazine, 3,5-difluorophenylhydrazine, Lipopolysaccharide, ovalbumin (from chicken egg white), fetuin (from fetal calf serum), trypsin and PNGase F were purchased from Sigma-Aldrich. Iodine, acetic acid (AcOH), ethyl acetate (EtOAc) and 2,3-naphthalenediamine were purchased from Merck Chemicals. A high-mannose type glycan containing nine mannoses (Mang), tetra-antennary N-glycan (NA4) and sialic acid containing tri-antennary N-glycan (A3) were purchased from QA-Bio Inc. Xyloglucan, maltohexose, sugar antigens (GM3, Gb5, Lewis Y, Lewis X, Globo H) were purchased from Elicityl (Crolles, France). NMR chiral shift reagents (Europium tri[3-(trifluoromethylhydroxymethylene)]-(+)-camphorate; Europium tri[3-(heptafluoropropylhydroxymethylene)]-(+)-camphorate; Europium tri[3-(heptafluoropropylhydroxymethylene)]-(+)-camphorate) were purchased from Sigma-Aldrich.

### Example 1

### Preparation and Characterization of New Isotope Labeled Compounds

Sugar-5FBIMs:

### (1R,2S,3R)-1-(5-fluoro-1H-benzo[d]imidazole-2-yl)butane-1,2,3,4-tetraol

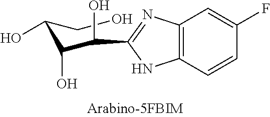

D-/L-arabinose (10 mg) and 4-fluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give AraFBIM. The supporting data is given below.

C₁₁H₁₃FN₂O₄; purple powder; ¹H NMR (MeOH-d4, 600 MHz) δ 7.55 (1H, dd, J=8.3 6.1 Hz, ArH), 7.29 (1H, d, J=8.9 Hz, ArH), 7.06 (1H, t, J=9.1 Hz, ArH), 5.31 (1 H, s, H1), 3.90 (1H, d, J=8.2 Hz, H2), 3.81 (2H, m, H4a, H4b), 3.68 (1H, dd, J=8.3, 2.6 Hz, H3); ¹³C NMR (MeOH-d4, 150 MHz) δ 161.7 (d, J_{F-C}=236.1 Hz), 159.6, 136.6 (d, J_{F-C}=13.5 Hz), 132.7, 116.5 (d, J_{F-C}=10.3 Hz), 113.5 (d, J_{F-C}=26.3 Hz), 101.7 (d, J_{F-C}=26.3 Hz), 75.3, 72.6, 68.9, 64.8; ¹⁹F NMR (MeOH-d4, 470 MHz) δ −122.3; Ms (MALDI-TOF) calcd for C₁₁H₁₃FN₂O₄: 256.086; found: m/z 256.922 [M+H]⁺.

### (1S,2R,3S, 4R)-1-(5-fluoro-1H-benzo[d]imidazole-2-yl)pentane-1,2,3,4-tetraol

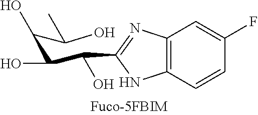

D-/L-fucose (10 mg) and 4-fluorophenyldiamine (10 mg) with catalytic amount of iodine (1 mg) in acetic acid (1 mL) was reacted at room temperature for 12 hours. The resulting solution was precipitated by ethyl acetate (30 mL) and centrifuged for three times to remove the excess reagent. The pellet was lyophilized to give FucFBIM. The supporting data is given below.

C₁₂H₁₅FN₂O₄; purple powder; ¹H NMR (MeOH-d4, 600 MHz) δ 7.61 (1H, dd, J=8.3, 3.8 Hz, ArH), 7.35 (1H, d, J=8.5 Hz, ArH), 7.15 (1H, t, J=8.8 Hz, ArH), 5.38 (1H, s, H1), 4.09 (1H, d, J=6.5 Hz, H2), 3.99 (1H, d, J=8.6 Hz, H4), 3.56 (1H, d, J=8.9 Hz, H3), 1.29 (3H, d, J=7.0 Hz, H5); ¹³C NMR (DMSO-d6, 150 MHz) δ 158.9, 157.7 (d, J_{F-C}=232.1 Hz), 135.6 (d, J_{F-C}=13.0 Hz), 132.5, 114.7 (d, J_{F-C}=10.5 Hz), 108.6 (d, J_{F-C}=25.1 Hz), 100.6 (d, J_{F-C}=26.5 Hz), 72.8, 72.1, 67.3, 64.9, 19.7; ¹⁹F NMR (MeOH-d4, 470 MHz) δ −122.0; MS (MALDI-TOF) calcd for C₁₂H₁₅FN₂O₄: 270.102; found: m/z 270.910 [M+H]⁺.

### (1S,2R,3S,4R)-1-(5-fluoro-1H-benzo[d]imidazole-2-yl)pentane-1,2,3,4,5-pentaol

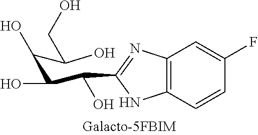

D-/L-galactose (10 mg) and 4-fluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GalFBIM. The supporting data is given below.

C₁₂H₁₅FN₂O₅; purple powder; ¹H NMR (H₂O-d2, 600 MHz) δ 7.69 (1H, dd, J=8.8, 4.5 Hz, ArH), 7.46 (1H, d, J=8.8 Hz, ArH), 7.26 (1H, t, J=8.8 Hz, ArH), 5.48 (1H, s, H1), 4.13 (1H, d, J=9.4 Hz, H2), 4.03 (1H, t, J=6.4 Hz, H4), 3.87 (1H, d, J=9.4, H3), 3.74 (2H, d, J=8.9 Hz, H5a, H5b); ¹³C NMR (H₂O-d2, 150 MHz) δ 160.2 (d, J_{F-C}=239.3 Hz), 156.36, 132.9 (d, J_{F-C}=13.5 Hz), 129.1, 115.2 (d, J_{F-C}=10.2 Hz), 113.7 (d, J_{F-C}=26.0 Hz), 100.6 (d, J_{F-C}=27.6 Hz), 72.3, 69.8, 69.0, 66.8, 63.1; ¹⁹F NMR (D₂O, 470 MHz) δ −117.5; MS (MALDI-TOF) calcd for C₁₂H₁₅FN₂O₅: 286.097; found: m/z 286.897 [M+H]⁺.

### (1S,2R,3S)-1-(5-fluoro-1H-benzo[d]imidazole-2-yl)-1,2,3,4-tetrtahvdroxy pentanoic acid

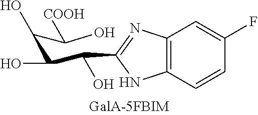

D-galactouronic acid (10 mg) and 4-fluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GalAFBIM. The supporting data is given below.

C₁₂H₁₃FN₂O₆; purple powder; ¹H NMR (D₂O, 600 MHz) δ 7.77 (1H, dd, J=9.1, 4.4 Hz, ArH), 7.55 (1H, dd, J=8.1, 1.7 Hz, ArH), 7.38 (1H, td, J=9.4, 1.9 Hz, ArH), 5.61 (1H, s, H1), 4.39 (1H, s, H4), 4.23 (1H, d, J=9.6 Hz, H3), 4.11 (1H, d, J=9.6, Hz, H2); ¹³C NMR (D₂O, 150 MHz) δ 178.2, 160.5 (d, J_{F-C}=240.7 Hz), 155.9, 131.5 (d, J_{F-C}=13.8 Hz), 127.7, 115.2 (d, J_{F-C}=10.3 Hz), 114.6 (d, J_{F-C}=26.2 Hz), 101.5 (d, J_{F-C}=28.0 Hz), 72.6, 70.9 (2×), 66.4; ¹⁹F NMR (D₂O, 470 MHz) δ−116.8; MS (MALDI-TOF) calcd for C₁₂H₁₃FN₂O₆: 300.076; found: m/z 300.889 [M+H]⁺.

### N-((1S,2R,3S,4R)-1-(5-fluoro-1H-benzo[d]imidazol-2-yl)-2,3,4,5-tetrahydroxypentyl)-acetamide

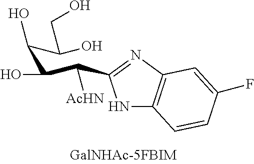

D-N-acetylgalactosamine (10 mg) and 4-fluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give Ga1NAcFBIM. The supporting data is given below.

C₁₄H₁₈FN₃O₅; brownish powder; ¹H NMR (D₂O, 600 MHz) δ 7.72 (1H, dd, J=9.0, 4.4 Hz, ArH), 7.50 (1H, dd, J=8.4, 2.5 Hz, ArH), 7.32 (1H, td, J=9.4, 2.4 Hz, ArH), 5.73 (1H, d, J=2.0 Hz, H1), 4.32 (1H, dd, J=9.5, 2.0 Hz, H2), 3.99 (1H, ddd, J=7.1, 5.9, 1.3 Hz, H4), 3.72-3.68 (2H, m, H5a, H5b), 3.67 (1H, dd, J=9.5, 1.3 Hz, H3), 2.20 (3H, s, Me); ¹³C NMR (D₂O, 150 MHz) δ 175.0, 160.3 (d, J_{F-C}=239.3 Hz), 153.7, 133.2 (d, J_{F-C}=12.8 Hz), 129.4, 115.3 (d, J_{F-C}=10.3 Hz), 113.8 (d, J_{F-C}=25.9 Hz), 100.6 (d, J_{F-C}=27.5 Hz), 70.8, 69.5, 69.3, 63.0, 49.7, 21.7; ¹⁹F NMR (D₂O, 470 MHz) δ −117.5; MS (MALDI-TOF) calcd for C₁₄H₁₈FN₃O₅: 327.123; found: m/z 312.820 [M−Me+H]⁺.

### (2R,3S,4R,5S)-5-amino-5-(5-fluoro-1H-benzo[d]imidazol-2-yl)pentane-1,2,3,4-tetraol

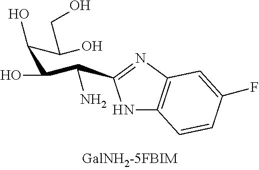

D-galactosamine (10 mg) and 4-fluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GalNH₂FBIM. The supporting data is given below.

C₁₂H₁₆FN₃O₄; brownish powder; ¹H NMR (D₂O, 600 MHz) δ 7.68 (1H, dd, J=9.0, 4.4 Hz, ArH), 7.48 (1H, dd, J=8.4, 2.5 Hz, ArH), 7.22 (1H, td, J=9.4, 2.4 Hz, ArH), 5.19 (1H, d, J=3.4 Hz, H5), 4.37 (1H, dd, J=7.9, 3.4 Hz, H4), 3.93 (1H, dd, J=7.7, 6.0 Hz, H2), 3.74 (1H, dd, J=8.0, 6.0 Hz, H3), 3.68 (2H, dd, J=11.6, 7.7 Hz, H1a, H1b); ¹³C NMR (D₂O, 150 MHz) δ 159.8 (d, J_{F-C}=235.8 Hz), 151.8, 130.9 (d, J_{F-C}=13.8 Hz), 127.1, 115.7 (d, J_{F-C}=10.4 Hz), 114.2 (d, J_{F-C}=26.0 Hz), 100.7 (d, J_{F-C}=28.3 Hz), 71.0, 70.0, 69.5, 62.7, 50.9; ¹⁹F NMR (D₂O, 470 MHz) δ −116.9; MS (MALDI-TOF) calcd for C₁₂H₁₆FN₃O₄: 285.113.

### (1S,2R,3R,4R)-1-(5-fluoro-1H-benzo[d]imidazole-2-yl)pentane-1,2,3,4,5-pentaol

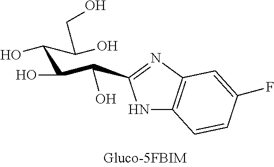

D-/L-glucose (10 mg) and 4-fluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GlcFBIM. The supporting data is given below.

C₁₂H₁₅FN₂O₅; black syrup; ¹H NMR (D₂O, 600 MHz) δ 7.79 (1H, dd, J=9.1, 4.4 Hz, ArH), 7.56 (1H, dd, J=8.4, 2.3 Hz, ArH), 7.40 (1H, td, J=9.1, 2.3 Hz, ArH), 5.46 (1H, d, J=5.2 Hz, H1), 4.42 (1H, d, J=5.2 Hz, H2), 3.84-3.70 (3H, m, H3, H4, H5a), 3.63 (1H, dd, J=11.8. 5.5 Hz, H5b); ¹³C NMR (D₂O, 150 MHz) δ 160.3 (d, J_{F-C}=239.8 Hz), 155.1, 132.1 (d, J_{F-C}=13.8 Hz), 128.3, 115.1 (d, J_{F-C}=10.3 Hz), 114.0 (d, J_{F-C}=26.1 Hz), 100.4 (d, J_{F-C}=27.8 Hz), 70.9, 70.7, 69.3, 67.7, 62.7; ¹⁹F NMR (D₂O, 470 MHz) δ −116.5; MS (MALDI-TOF) calcd for C₁₂H₁₅FN₂O₅: 286.097; found: m/z 286.913 [M+H]⁺.

### (1S,2R,3R)-1-(5-fluoro-1H-benzo[d]imidazole-2-yl)-1,2,3,4-tetrtahydroxy pentanoic acid

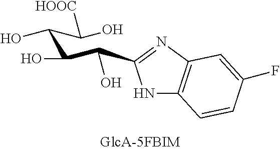

D-glucouronic acid (10 mg) and 4-fluorophenyldiamine (10 mg) with catalytic amount of iodine (1 mg) in acetic acid (1 mL) was mixed at room temperature for 12 hour. The resulting solution was precipitated by ethyl acetate (30 mL) and centrifuged for three times to remove the excess reagent. The pellet was lyophilized to give GlcAFBIM. The supporting data is given below.

C₁₂H₁₃FN₂O₆; black powder; ¹H NMR (D₂O, 600 MHz) δ 7.76 (1H, dd, J=9.0, 4.3 Hz, ArH), 7.53 (1H, dd, J=8.4, 2.3 Hz, ArH), 7.37 (1H, td, J=9.4, 2.3 Hz, ArH), 5.44 (1H, d, J=4.4 Hz, H1), 4.30 (1H, dd, J=4.4, 3.6 Hz, H2), 4.21 (1H, d, 5.0 Hz, H4), 4.04 (1H, dd, J=5.0, 3.6 Hz, H3); ¹³C NMR (D₂O, 150 MHz) δ 177.6, 160.5 (d, J_{F-C}=240.5 Hz), 154.8, 131.4 (d, J_{F-C}=13.87 Hz), 127.5, 115.1 (d, J_{F-C}=10.4 Hz), 114.5 (d, J_{F-C}=26.2 Hz), 100.5 (d, J_{F-C}=28.1 Hz), 73.2, 72.0, 70.8, 67.2; ¹⁹F NMR (D₂O, 470 MHz) δ −116.2; MS (MALDI-TOF) calcd for C₁₂H₁₃FN₂O₆: 300.076; found: m/z 300.894 [M+H]⁺.

### N-((1S,2R,3R,4R)-1-(5-fluoro-1H-benzo[d]imidazol-2-yl)-2,3,4,5-tetrahydroxypentyl)-acetamide

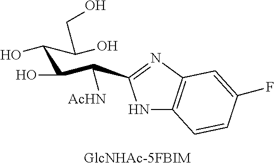

D-N-acetylglucosamine (10 mg) and 4-fluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GlcNAcFBIM. The supporting data is given below.

C₁₄H₁₈FN₃O₅; black syrup; NMR (D₂O, 600 MHz) δ 7.74 (1H, dd, J=9.0, 4.4 Hz, ArH), 7.51 (1H, d, J=8. 5 Hz, ArH), 7.34 (1H, td, J=9.3, 2.4 Hz, ArH), 5.50 (1H, d, J=5.3 Hz, H1), 4.47 (1H, d, J=5.2 Hz, H2), 3.95-3.77 (2H, m, H3, H4), 3.61 (1H, dd, J=11.7, 5.9 Hz, H5a), 3.49 (1H, d, J=8.5 Hz, H5b), 2.15 (3H, s, Me); ¹³C NMR (D₂O, 150 MHz) δ 174.8, 160.2 (d, J_{F-C}=239.5 Hz), 152.5, 132.9 (d, J_{F-C}=13.6 Hz), 129.2, 115.3 (d, J_{F-C}=10.4 Hz), 113.8 (d, J_{F-C}=26.0 Hz), 100.5 (d, J_{F-C}=27.5 Hz), 70.8, 70.5, 69.8, 62.6, 51.2, 21.9; ¹⁹F NMR (D₂O, 470 MHz) δ −116.6; MS (MALDI-TOF) calcd for C₁₄H₁₈FN₃O₅: 327.123.

### (2R,3R,4R,5S)-5-amino-5-(5-fluoro-1H-benzo[d]imidazol-2-yl)pentane-1,2,3,4-tetraol

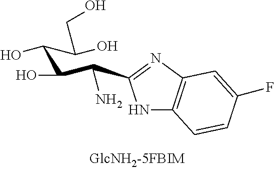

D-glucosamine (10 mg) and 4-fluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GlcNH2FBIM. The supporting data is given below.

C₁₂H₁₆FN₃O₄; brownish powder; ¹H NMR (D₂O, 600 MHz) δ 7.69 (1H, dd, J=9.0, 4.4 Hz, ArH), 7.45 (1H, dd, J=8.4, 2.5 Hz, ArH), 7.21 (1H, td, J=9.4, 2.4 Hz, ArH), 4.93 (1H, d, J=8.0 Hz, H5), 4.59 (1H, d, J=8.0 Hz, H4), 3.79 (2H, m, H2, H1a), 3.57 (1H, dd, J=12.0, 6.1 Hz, H1b), 3.31 (1H, d, J=9.5 Hz, H3); ¹³C NMR (D₂O, 150 MHz) δ 159.7 (d, J_{F-C}=236.1 Hz), 155.1, 134.0 (d, J_{F-C}=13.8 Hz), 130.2, 116.4 (d, J_{F-C}=9.3 Hz), 114.6 (d, J_{F-C}=26.1 Hz), 100.4 (d, J_{F-C}=25.9 Hz), 70.4, 70.0, 69.4, 62.7, 51.6; ¹⁹F NMR (D₂O, 470 MHz) δ −116.1; MS (MALDI-TOF) calcd for C₁₂H₁₆FN₃O₄: 285.113.

### (1R,2R,3R,4R)-1-(5-fluoro-1H-benzo[d]imidazole-2-yl)pentane-1,2,3,4,5-pentaol

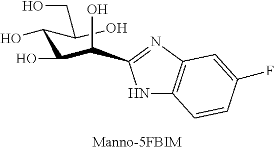

D-/L-mannose (10 mg) and 4-fluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give ManFBIM. The supporting data is given below.

C₁₂H₁₅FN₂O₅; purple powder; ¹H NMR (D₂O, 600 MHz) δ 7.71 (1H, dd, J=9.0, 4.5 Hz, ArH), 7.48 (1H, dd, J=8.7, 2.3 Hz, ArH), 7.29 (1H, td, J=9.1, 2.3 Hz, ArH), 5.16 (1H, d, J=8.5 Hz, H1), 4.20 (1H, d, J=8.5 Hz, H2), 3.92-3.87 (2H, m, H3, H5a), 3.81 (1H, ddd, J=11.8, 6.1, 2.9 Hz, H4), 3.71 (1H, dd, J=11.8, 6.1 Hz, H5b); ¹³C NMR (D₂O, 150 MHz) δ 160.2 (d, J_{F-C}=238.6 Hz), 155.6, 133.5 (d, J_{F-C}=13.8 Hz), 129.8, 115.4 (d, J_{F-C}=10.3 Hz), 113.4 (d, J_{F-C}=25.8 Hz), 100.6 (d, J_{F-C}=27.3 Hz), 71.0, 70.6, 69.0, 66.8, 63.1; ¹⁹F NMR (D₂O, 470 MHz) δ −119.6; HRMS (ESI) calcd for C₁₂H₁₅FN₂O₅: 286.097.

### N-((1R,2R,3R,4R)-1-(5-fluoro-1H-benzo[d]imidazol-2-yl)-2,3,4,5-tetrahydroxypentyl)-acetamide

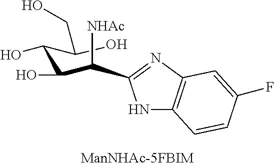

D-N-acetylmannosamine (10 mg) and 4-fluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give ManNAcFBIM. The supporting data is given below.

C₁₄H₁₈FN₃O₅; brownish powder; ¹H NMR (D₂O, 600 MHz) δ 7.71 (1H, dd, J=9.0, 4.4 Hz, ArH), 7.48 (1H, dd, J=8.5, 2.2 Hz, ArH), 7.31 (1H, td, J=9.4, 2.1 Hz, ArH), 5.45 (1H, d, J=8.2 Hz, H1), 4.46 (1H, d, J=8.2 Hz, H2), 3.86 (1H, dd, J=11.9, 2.6 Hz, H5a), 3.79 (1H, ddd, J=8.2, 4.9, 2.6 Hz, H4), 3.72 (1H, d, J=8.2 Hz, H3), 3.68 (1H, dd, J=11.9, 4.9 Hz, H5b), 2.12 (3H, s, Me); ¹³C NMR (D₂O, 150 MHz) δ 174.5, 160.4 (d, J_{F-C}=239.8 Hz), 156.7, 132.3 (d. J_{F-C}=13.7 Hz), 128.5, 115.3 (d, J_{F-C}=10.2 Hz), 114.2 (d, J_{F-C}=26.1 Hz), 100.5 (d, J_{F-C}=27.8 Hz), 70.4, 69.3, 69.2, 62.9, 49.5, 21.7; ¹⁹F NMR (D₂O, 470 MHz) δ −115.3; MS (MALDI-TOF) calcd for C₁₄H₁₈FN₃O₅: 327.123.

### (1S,2R,3R,4R)-1-(5-fluoro-1H-benzo[d]imidazol-2-yl)-3-O-(2′,3′,4′,5′-tetrahydroxy-α-D-glucopyranosyl)pentane-1,2,4,5-tetraol

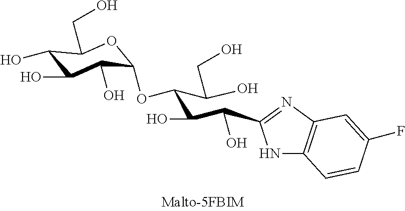

Maltose (10 mg) and 4-fluorophenyldiamine (10 mg) with catalytic amount of iodine (1 mg) in acetic acid (1 mL) was mixed at room temperature for 12 hour. The resulting solution was precipitated by ethyl acetate (30 mL) and centrifuged for three times to remove the excess reagent. The pellet was lyophilized to give MaltoFBIM. The supporting data is given below.

C₁₈H₂₅FN₂O₁₀; black syrup; ¹H NMR (D₂O, 600 MHz) δ 7.71 (1H, dd, J=8.9, 4.6 Hz, ArH), 7.48 (1H, dd, J=8.6, 2.3 Hz, ArH), 7.30 (1H, td, J=9.5, 2.3 Hz, ArH), 5.46 (1H, d, J=3.2 Hz, H1′), 5.18 (1H, d, J=3.9 Hz, H1), 4.38 (1H, dd, J=5.3, 3.5 Hz, H2), 4.09 (1H, d, J=7.0 Hz), 3.98 (1H, dd, J=5.0, 4.4 Hz), 3.91-3.61 (7H, m), 3.45 (1H, t, J=9.6 Hz); ¹³C NMR (D₂O, 150 MHz) δ 160.2 (d, J_{F-C}=239.6 Hz), 154.8, 133.1 (d, J_{F-C}=11.6 Hz), 129.3, 115.4 (d, J_{F-C}=10.2 Hz), 113.8 (d, J_{F-C}=26.0 Hz), 100.7 (d, J_{F-C}=27.6 Hz), 100.4, 80.6, 72.8, 72.6, 72.5, 72.4, 71.6, 69.3, 67.7, 62.2, 60.3; ¹⁹F NMR (D₂O, 470 MHz) δ −117.7; HRMS (ESI) calcd for C₁₈H₂₅FN₂O₁₀: 448.149.

### Maltotrio-5FBIM

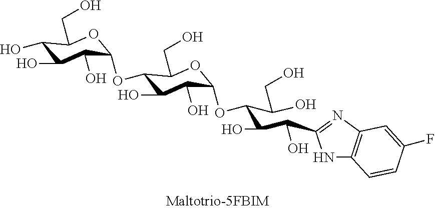

Maltotriose (10 mg) and 4-fluorophenyldiamine (10 mg) with catalytic amount of iodine (1 mg) in acetic acid (1 mL) was mixed at room temperature for 12 hour. The resulting solution was precipitated by ethyl acetate (30 mL) and centrifuged for three times to remove the excess reagent. The pellet was lyophilized to give maltotrioFBIM. The supporting data is given below.

C₂₄H₃₅FN₂O₁₅; brownish powder; ¹H NMR (D₂O, 600 MHz) δ 7.66 (1H, dd, J=8.9, 4.4 Hz, ArH), 7.43 (1H, dd, J=8.8, 2.0 Hz, ArH), 7.23 (1H, td, J=9.0, 1.9 Hz, ArH), 5.40 (1H, d, J=3.9 Hz, H1′), 5.37 (1H, d, J=3.8 Hz, H1″), 5.16 (1H, d, J=3.9 Hz, H1), 4.37 (1H, d, J=4.4 Hz, H2), 4.08 (1H, dd, J=6.9, 3.4 Hz), 3.96-3.58 (20H, m), 3.43 (1H, t, J=9.5 Hz); ¹³C NMR (D₂O, 150 MHz) δ 159.9 (d, J_{F-C}=237.1 Hz), 155.1, 135.1 (d, J_{F-C}=11.6 Hz), 131.3, 115.5 (d, J_{F-C}=10.8 Hz), 112.7 (d, J_{F-C}=26.0 Hz), 100.7 (d, J_{F-C}=25.8 Hz), 100.2, 99.8, 80.7, 76.8, 73.2, 73.1, 72.9, 72.7, 72.5, 71.7, 71.3, 71.0, 69.3, 68.0, 62.2, 60.3 (2×); ¹⁹F NMR (MeOH-d4, 470 MHz) δ −117.9; HRMS (ESI) calcd for C₂₄H₃₅FN₂O₁₅: 610.202.

### Maltohexao-5FBIM

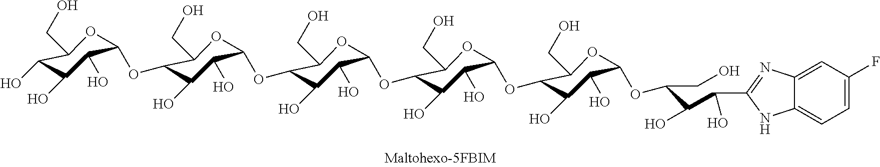

Maltohexose (10 mg) and 4-fluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give MaltohexoFBIM. The supporting data is given below.

C₄₂H₆₅FN₂O₃₀; brownish powder; ¹H NMR (D₂O, 600 MHz) δ 7.72 (1H, dd, J=9.0, 4.5 Hz, ArH), 7.49 (1H, dd, J=6.4, 2.0 Hz, ArH), 7.31 (1H, td, J=9.2, 1.9 Hz, ArH), 5.43 (1H, d, J=3.8 Hz, H1′), 5.41 (2H, m, H1″, H1′″), 5.40 (1H, m, H1″″), 5.37 (1H, d, J=3.9 Hz, H1″″′), 5.16 (1H, d, J=4.0 Hz, H1), 4.39 (1H, dd, J=8.0, 4.2 Hz, H2), 4.09-3.58 (28H, m), 3.43 (1H, t, J=9.4 Hz); ¹³C NMR (D₂O, 150 MHz) δ 160.2 (d, J_{F-C}=238.6 Hz), 155.0, 135.1 (d, J_{F-C}=11.6 Hz), 131.0, 115.4 (d, J_{F-C}=10.6 Hz), 113.4 (d, J_{F-C}=27.2 Hz), 100.7 (d, J_{F-C}=27.2 Hz), 100.1, 99.7 (3×), 99.6, 80.7, 77.0, 76.9, 76.8, 76.7, 76.6, 73.3 (2×), 73.2, 73.1 (2×), 72.8, 72.7 (2×), 72.4, 71.7, 71.5 (2×), 71.4 (2×), 71.3 (2×), 71.2, 70.9, 69.3, 67.8, 62.2, 60.5 (3×), 60.4, 60.2; ¹⁹F NMR (MeOH-d4, 470 MHz) δ −117.0; HRMS (ESI) calcd for C₄₂H₆₅FN₂O₃₀: 1096.361.

### (1R,2R,3R,4R)-1-(5-fluoro-1H-benzo[d]imidazole-2-yl)pentane-1,2,3,4-tetraol

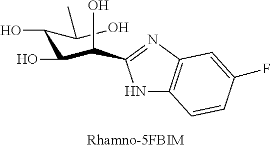

D-/L-rhamnose (10 mg) and 4-fluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give RhaFBIM. The supporting data is given below.

C₁₂H₁₅FN₂O₄; black powder; ¹H NMR (D₂O, 600 MHz) δ 7.77 (1H, dd, J=9.1, 4.4 Hz, ArH), 7.54 (1H, dd, J=8.5, 2.3 Hz, ArH), 7.38 (1H, td, J=9.4, 2.3 Hz, ArH), 5.23 (1H, d, J=8.6 Hz, H1), 4.17 (1H, dd, J=8.8, 0.8 Hz, H2), 3.91 (1H, dd, J=8.0, 6.3 Hz, H4), 3.71 (1H, d, J=8.2 Hz, H3), 1.31 (3H, d, J=6.3 Hz, H5); ¹³C NMR (DMSO-d6, 150 MHz) δ 160.6 (d, J_{F-C}=240.6 Hz), 155.2, 131.5 (d, J_{F-C}=13.7 Hz), 127.7, 115.2 (d, J_{F-C}=10.2 Hz), 114.6 (d, J_{F-C}=25.3 Hz), 100.5 (d, J_{F-C}=28.1 Hz), 73.0, 70.9, 66.7, 66.4, 19.1; ¹⁹F NMR (MeOH-d4, 470 MHz) δ −116.8; HRMS (ESI) calcd for C₁₂H₁₅FN₂O₄: 270.102.

### (1S,2S,3R)-1-(5-fluoro-1H-benzo[d]imidazole-2-yl)butane-1,2,3,4-tetraol

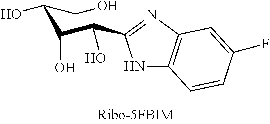

D-/L-ribose (10 mg) and 4-fluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give RibFBIM. The supporting data is given below.

C₁₁H₁₃FN₂O₄; black syrup; ¹H NMR (D₂O, 600 MHz) δ 7.78 (1H, dd, J=9.1, 4.5 Hz, ArH), 7.55 (1H, dd, J=8.2, 2.3 Hz, ArH), 7.39 (1H, td, J=9.4, 2.3 Hz, ArH), 5.47 (1H, d, J=4.5 Hz, H1), 4.13 (1H, dd, J=7.4, 4.4 Hz, H2), 3.79 (1H, dd, J=9.2, 5.0 Hz, H4a), 3.74-3.69 (2H, m, H3, H4b); ¹³C NMR (D₂O, 150 MHz) δ 160.6 (d, J_{F-C}=241.1 Hz), 153.9, 131.2 (d, J_{F-C}=14.2 Hz), 127.3, 115.3 (d,J_{F-C}=10.3 Hz), 114.8 (d, J_{F-C}=26.1 Hz), 100.5 (d, J_{F-C}=28.0 Hz), 72.8, 71.0, 67.4, 62.5; ¹⁹F NMR (MeOH-d4, 470 MHz) δ −115.8; HRMS (ESI) calcd for C₁₁H₁₃FN₂O₄: 256.086.

### (1S,2R,3R)-1-(5-fluoro-1H-benzo[d]imidazole-2-yl)butane-1,2.3,4-tetraol

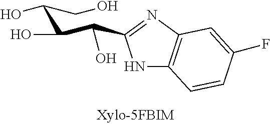

D-/L-xylose (10 mg) and 4-fluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give XylFBIM. The supporting data is given below.

C₁₁H₁₃FN₂O₄; black syrup; ¹H NMR (D₂O, 600 MHz) δ 7.76 (1H, dd, J=9.0, 4.3 Hz, ArH), 7.53 (1H, dd, J=8.3, 2.2 Hz, ArH), 7.37 (1H, td, J=9.4, 2.4 Hz, ArH), 5.42 (1H, d, J=4.1 Hz, H1), 4.15 (1H, t, J=4.1 Hz, H2), 3.92 (1H, ddd, J=8.8, 6.5, 4.5 Hz, H3), 3.75 (1H, dd, J=11.8, 4.9 Hz, H4a), 3.69 (1H, dd, J=11.8, 6.6 Hz, H4b); ¹³C NMR (D₂O, 150 MHz) δ 160.5 (d, J_{F-C}=240.7 Hz), 155.0, 131.6 (d, J_{F-C}=13.5 Hz), 127.7, 115.2 (d, J_{F-C}=10.3 Hz), 114.5 (d, J_{F-C}=26.1 Hz), 100.5 (d, J_{F-C}=28.1 Hz), 72.1, 70.4, 67.2, 62.4; ¹⁹F NMR (MeOH-d4, 470 MHz) δ −117.0; HRMS (ESI) calcd for C₁₁H₁₃FN₂O₄: 256.086.

### N-((2R,3R,4R,5R,6R)-1-(6-fluoro-3-oxo-3,4-dihydroquinoxalin-2-yl)-2,4,5,6,7-pentahydroxyheptan-3-yl)acetamide

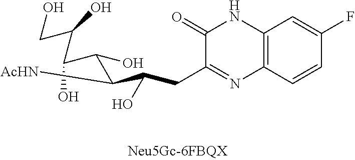 [SMILES: CC(=O)N[C@@H](C(O)[C@H](O)[C@H](O)CO)[C@H](O)Cc1nc2ccc(F)cc2[nH]c1=O]

Sialic acid (Neu5Ac; 10 mg) and 4-fluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give Neu5AcFBQX (SiaFBQX). The supporting data is given below.

C₁₇H₂₂FN₃O₇; purple powder; ¹H NMR (DMSO-d6, 600 MHz) δ 7.75 (1H, dd, J=8.9, 5.8 Hz, ArH), 7.12 (1H, td, J=8.8, 2.8 Hz, ArH), 7.07 (1H, dd, J=9.5, 2.7 Hz, ArH), 4.40 (1H, d, J=5.4 Hz, H2), 4.28 (1H, t, J=5.4 Hz, H5), 3.77-3.70 (2H, m, H7a, H7b), 3.59 (1H, dd, J=8.8, 5.4 Hz, H4), 3.50 (1H, m, H6), 3.19 (1H, dd, J=8.0, 5.9 Hz, H3), 2.92 (1H, dd, J=14.2, 9.2 Hz, H1a). 2.74 (1H, dd, J=14.2, 4.5 Hz, H1b), 1.95 (3H, s, Me); ¹³C NMR (DMSO-d6, 150 MHz) δ 171.2, 161.8 (d, J_{F-C}=244.9 Hz), 159.1, 154.9, 130.4 (d, J_{F-C}=10.6 Hz), 128.8, 110.7 (d, J_{F-C}=3.7 Hz), 101.0 (d, J_{F-C}=25.9 Hz), 70.9, 70.1, 67.9, 66.0, 63.9, 54.1, 38.4, 22.5; ¹⁹F NMR (D₂O, 470 MHz) δ −109.3; HRMS (ESI) calcd for C₁₇H₂₂FN₃O₇: 399.144.

### N-((2R,3R,4R,5R,6R)-1-(6-fluoro-3-oxo-3,4-dihydroquinoxalin-2-yl)-2,4,5,6,7-pentahydroxyheptan-3-yl)-2-hydroxyacetamide

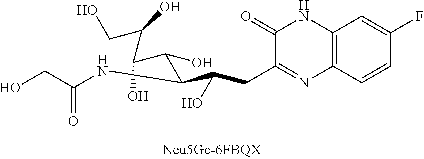 [SMILES: O=C(CO)N[C@@H](C(O)[C@H](O)[C@H](O)CO)[C@H](O)Cc1nc2ccc(F)cc2[nH]c1=O]

Neu5Gc (10 mg) and 4-fluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give Neu5GcFBQX. The supporting data is given below.

C₁₇H₂₂FN₃O₈; black powder; ¹H NMR (D₂O, 600 MHz) δ 7.81 (1H, dd, J=8.9, 5.5 Hz, ArH), 7.20 (1H, td, J=8.8, 2.5 Hz, ArH), 7.11 (1H, dd, J=9.1, 2.5 Hz, ArH), 4.20 (1H, dd, J=9.0, 4.0 Hz, H2), 4.17 (2H, s, CH₂), 4.13 (1H, d, J=9.2 Hz, H5), 4.06 (1H, dd, J=10.4, 4.1 Hz, H4), 3.85 (1H, dd, J=11.8, 2.6 Hz, H7a), 3.77 (1H, ddd, J=8.9, 6.2, 2.8 Hz, H6), 3.65 (1H, dd, J=11.8, 6.2 Hz, H7b), 3.49 (1H, d, J=9.3 Hz, H3), 3.06 (1H, dd, J=14.2, 4.2 Hz, H1a), 3.03 (1H, dd, J=14.2, 8.5 Hz, H1b); ¹³C NMR (DMSO-d6, 150 MHz) δ 171.2, 161.8 (d, J_{F-C}=244.9 Hz), 159.1, 154.9, 130.4 (d, J_{F-C}=10.6 Hz), 128.8, 110.7 (d, J_{F-C}=23.7 Hz), 101.0 (d, J_{F-C}=25.9 Hz), 70.9, 70.1, 67.9, 66.0, 63.9, 54.1, 38.4, 22.5; ¹⁹F NMR (D₂O, 470 MHz) δ −109.2; HRMS (ESI) calcd for C₁₇H₂₂FN₃O₈: 415.139.

### 7-fluoro-3-((2R,3R,4R,5R,6R)-2,3,4,5,6,7-hexahydroxyheptyl)quinoxalin-2(1H)-one

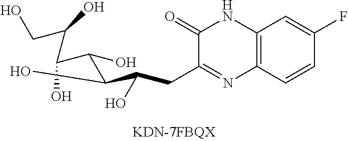 [SMILES: O=c1[nH]c2cc(F)ccc2nc1C[C@@H](O)[C@@H](O)C(O)[C@H](O)[C@H](O)CO]

KDN (10 mg) and 4-fluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give KDNFBQX. The supporting data is given below.

C₁₅H₁₉FN₂O₇; purple powder; ¹H NMR (D₂O, 600 MHz) δ 7.86 (1H, dd, J=9.0, 5.5 Hz, ArH), 7.22 (1H, td, J=8.8, 2.5 Hz, ArH), 7.15 (1H, dd, J=9.2, 2.5 Hz, ArH), 4.54 (1H, ddd, J=9.0, 8.5, 4.5 Hz, H2), 3.97 (1H, d, J=9.5 Hz, H4), 3.88 (1H, dd, J=11.8, 2.6 Hz, H7a), 3.85 (1H, d, J=8.9 Hz, H3), 3.79 (1H, td, J=6.2, 2.6 Hz, H6), 3.70 (1H, d, J=9.0 Hz, H5), 3.68 (1H, dd, J=11.6, 6.1 Hz, H7b), 3.21 (1H, dd, J=14.0, 4.7 Hz, H1a), 3.16 (1H, dd, J=14.0, 4.6 Hz, H1b); ¹³C NMR (DMSO-d6, 150 MHz) δ 162.5 (d, J_{F-C}=247.0 Hz), 159.8, 154.9, 130.6 (d, J_{F-C}=10.6 Hz), 128.8 (d, J_{F-C}=10.6 Hz), 128.0, 110.8 (d, J_{F-C}=23.1 Hz), 101.0 (d, J_{F-C}=26.3 Hz), 71.6, 71.5, 69.7, 68.7, 67.9, 63.9, 37.7; ¹⁹F NMR (D₂O, 470 MHz) δ −109.4; HRMS (ESI) calcd for C₁₅H₁₉FN₂O₇: 358.118.

### 7-fluoro-3-((2R,3R,4R,5R)-2,3,4,5,6-pentahydroxyheptyl)quinoxalin-2(1H)-one

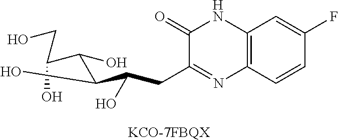

KDO (10 mg) and 4-fluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give KDOFBQX. The supporting data is given below.

C₁₄H₁₇FN₂O₆; brownish powder, ¹H NMR (D₂O, 600 MHz) δ 7.77 (1H, dd, J=9.0, 5.6 Hz, ArH), 7.17 (1H, td, J=8.8, 2.6 Hz, ArH), 7.06 (1H, dd, J=9.2, 2.5 Hz, ArH), 4.29 (1H, ddd, J=9.0, 3.7, 3.5 Hz, H2), 3.89 (2H, dd, J=8.8, 2.9 Hz, H6a, H6b), 3.84-3.77 (2H, m, H3, H5), 3.69 (1H, dd, J=11.0, 6.4 Hz, H4), 3.37 (1 H, dd, J=14.5, 3.5 Hz, H1a), 2.98 (1H, dd, J=14.5, 9.3 Hz, H1b); ¹³C NMR (D₂O, 150 MHz) δ 162.9 (d, J_{F-C}=247.8 Hz), 158.0, 156.5, 132.0 (d, J_{F-C}=12.7 Hz), 129.7 (d, J_{F-C}=10.6 Hz), 128.9, 112.8 (d, J_{F-C}=24.2 Hz), 101.9 (d, J_{F-C}=26.6 Hz), 72.3, 70.9, 69.2, 69.0, 63.1, 37.5; ¹⁹F NMR (D₂O, 470 MHz) δ −109.8; HRMS (ESI) calcd for C₁₄H₁₇FN₂O₆: 328.107.

### Sugar-5,6F₂BIMs:

(1R,2S,3R)-1-(5,6-difluoro-1H-benzo[d]imidazole-2-yl)butane-1,2,3,4-tetraol

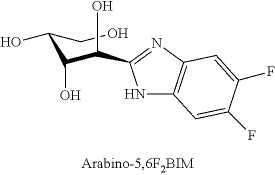

D-/L-arabinose (10 mg) and 4,5-difluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give AraF₂BIM. The supporting data is given below.

C₁₁H₁₂F₂N₂O₄; purple powder; [α]²⁵_{D}−274.2 (c 0.025, DMSO); ¹H NMR (DMSO-d6, 600 MHz) δ 7.72 (1H, t, J=8.2 Hz, ArH), 5.57 (1H, d, J=1.9 Hz, H1), 4.01 (1H, dd, J=9.0, 1.9 Hz, H2), 3.93 (1H, ddd, J=9.0, 5.6, 2.6 Hz, H3), 3.89 (1H, dd, J=12.0, 2.6 Hz, H4a), 3.75 (1H, dd, J=12.0, 5.6 Hz, H4b); ¹³C NMR (MeOH-d4, 150 MHz) δ 160.1, 151.0 (d, J_{F-C}=246.8 Hz), 150.9 (d, J_{F-C}=246.6 Hz), 129.4 (2×), 103.7 (d, J_{F-C}=6.8 Hz), 103.6 (d, J_{F-C}=6.6 Hz), 75.0, 72.4, 68.8, 64.6; ¹⁹F NMR (D₂O, 470 MHz) δ −142.9; MS (MALDI-TOF) calcd for C₁₁H₁₂F₂N₂O4: 274.077; found: m/z 274.922 [M+H]⁺.

### (1S,2R,3S,4R)-1-(5,6-difluoro-1H-benzo[d]imidazole-2-yl)pentane-1,2,3,4-tetraol

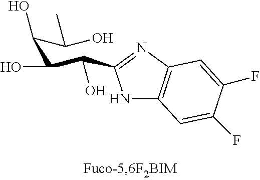

D-/L-fucose (10 mg) and 4,5-difluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give FucF₂BIM. The supporting data is given below.

C₁₂H₁₄F₂N₂O₄; purple powder; [α]²⁵_{D}−55.3 (c 0.025, DMSO); ¹H NMR (DMSO-d6, 600 MHz) δ 7.47 (2H, brs, ArH), 5.52 (1H, d, J=6.4 Hz, H1), 5.10 (1H, d, J=5.8 Hz, H3), 4.54 (1H, d, J=7.0 Hz, H2), 4.40 (1H, d, J=5.9 Hz, OH), 4.18 (1H, brs, OH), 3.91 (1H, brs, OH), 3.86 (1H, t, J=7.5 Hz, H4), 1.12 (3H, d, J=6.5 Hz, H5); ¹³C NMR (DMSO-d6, 150 MHz) δ 159.6, 145.8 (d, J_{F-C}=234.9 Hz), 145.7 (d, J_{F-C}=234.9 Hz),129.3 (d, J_{F-C}=6.3 Hz), 128.9 (d, J_{F-C}=6.6 Hz), 102.5 (d, J_{F-C}=6.7 Hz), 102.3 (d, J_{F-C}=7.0 Hz), 72.8, 72.1, 67.3, 64.8, 19.7; ¹⁹F NMR (D₂O, 470 MHz) δ −140.0; MS (MALDI-TOF) calcd for C₁₂H₁₄F₂N₂O₄: 288.092; found: m/z 288.955 [M+H]⁺.

### (1S,2R,3S,4R)-1-(5,6-difluoro-1H-benzo[d]imidazol-2-yl)pentane-1,2,3,4,5-pentaol

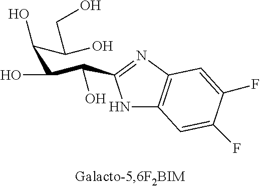

D-/L-galactose (10 mg) and 4,5-difluorophenyldiamine (10 mg) with catalytic amount of iodine (1 mg) in acetic acid (1 mL) was mixed at room temperature for 12 hour. The resulting solution was precipitated by ethyl acetate (30 mL) and centrifuged for three times to remove the excess reagent. The pellet was lyophilized to give GalF₂BIM. The supporting data is given below.

C₁₂H₁₄F₂N₂O₅; brownish powder; [α]²⁵_{D}+50.6 (c 0.025, DMSO); ¹H NMR (D₂O, 600 MHz) δ 7.70 (2H, td, J=8.1, 3.3 Hz, ArH), 5.58 (1H, d, J=1.5 Hz, H1), 4.12 (1H, dd, J=9.4, 1.5 Hz, H2), 4.01 (1H, td, J=6.6, 0.8 Hz, H4), 3.88 (1H, dd, J=9.4, 0.8 Hz, H3), 3.73 (2H, d, J=6.5 Hz, H5a, H5b); ¹³C NMR (D₂O, 150 MHz) δ 156.7, 149.4 (d, J_{F-C}=249.7 Hz), 149.3 (d, J_{F-C}=249.6 Hz), 127.5 (d, J_{F-C}=6.3 Hz), 127.5 (d, J_{F-C}=6.6 Hz), 102.5 (d, J_{F-C}=6.2 Hz), 102.4 (d, J_{F-C}=6.8 Hz), 72.3, 69.8, 68.9, 66.7, 63.0; ¹⁹F NMR (D₂O, 470 MHz) δ 140.5; MS (MALDI-TOF) calcd for C₁₂H₁₄F₂N₂O₅: 304.087; found: m/z 304.983 [M+H]⁺.

### (1S,2R,3S)-1-(5,6-difluoro-1H-benzo[d]imidazole-2-yl)-1,2,3,4-tetrtahydroxy pentanoic acid

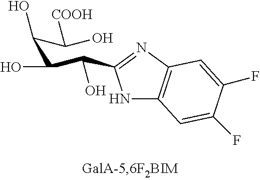

Galacturonic acid (10 mg) and 4,5-difluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GalAF₂BIM. The supporting data is given below.

C₁₂H₁₂F₂N₂O₆; purple powder; [α]²⁵_{D}−13.2 (c 0.025, DMSO); ¹H NMR (D₂O, 600 MHz) δ 7.72 (2H, t, J=8.2 Hz, ArH), 5.59 (1H, d, J=1.7 Hz, H1), 4.46 (1H, s, H4), 4.24 (1H, d, J=9.7 Hz, H3), 4.11 (1H, dd, J=9.7, 1.7 Hz, H2); ¹³C NMR (D₂O, 150 MHz) δ 177.7, 156.4, 149.4 (d, J_{F-C}=245.8 Hz), 149.3 (d, J_{F-C}=245.8 Hz), 127.1 (d, J_{F-C}=6.3 Hz), 127.0 (d, J_{F-C}=6.6 Hz), 102.5 (d, J_{F-C}=6.7 Hz), 102.3 (d, J_{F-C}=7.0 Hz), 72.4, 70.8, 70.6, 66.5; ¹⁹F NMR (D₂O, 470 MHz) δ −139.6; MS (MALDI-TOF) calcd for C₁₂H₁₂F₂N₂O₆: 318.066; found: m/z 318.923 [M+H]⁺.

### N-((1S,2R,3S,4R)-1-(5,6-difluoro-1H-benzo[d]imidazol-2-yl)-2,3,4,5-tetrahydroxypentyl)-acetamide

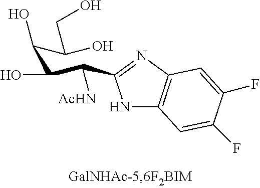

D-N-acetylgalactosamine (10 mg) and 4,5-difluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GalNAcF₂BIM. The supporting data is given below.

C₁₄H₁₇F₂N₃O₅; black powder; [α]²⁵_{D}−4.0 (c 0.010, DMSO); ¹H NMR (D₂O, 600 MHz) δ 7.68 (2H, t, J=8.2 Hz, ArH), 5.70 (1H, d, J=1.3 Hz, H1), 4.31 (1H, dd, J=9.5, 1.3 Hz, H2), 3.98 (1H, t, J=6.5 Hz, H4), 3.76-3.65 (3H, m, H3, H5a, H5b), 2.19 (3H, s, Me); ¹³C NMR (D₂O, 150 MHz) δ 174.9, 155.0, 149.1 (d, J_{F-C}=248.0 Hz), 149.0 (d, J_{F-C}=247.9 Hz), 128.9 (d, J_{F-C}=6.3 Hz), 127.5 (d, J_{F-C}=6.5 Hz), 102.5 (d, J_{F-C}=6.2 Hz), 102.3 (d, J_{F-C}=6.8 Hz), 72.2, 70.4, 69.7, 62.4, 49.8, 21.7; ¹⁹F NMR (D₂O, 470 MHz) δ −139.9/−141.1; MS (MALDI-TOF) calcd for C₁₄H₁₇F₂N₃O₅: 345.114; found: m/z 368.004 [M+Na]⁺.

### (2R,3S,4R,5S)-5-amino-5-(5,6-difluoro-1H-benzo[d]imidazol-2-yl)pentane-1,2,3,4-tetraol

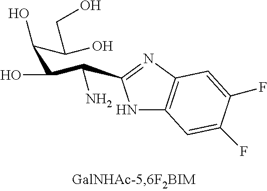

D-galactosamine (10 mg) and 4,5-difluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GalNH₂F₂BIM. The supporting data is given below.

C₁₂H₁₅F₂N₃O₄; brownish powder; ¹H NMR (D₂O, 600 MHz) δ 7.58 (2H, td, J=8.6 Hz, ArH), 5.11 (1H, d, J=3.5 Hz, H5), 4.35 (1H, dd, J=8.1, 3.4 Hz, H4), 3.92 (1H, dd, J=11.4, 6.0 Hz, H1a), 3.78-3.66 (3H, m, H3, H2, H1b); ¹³C NMR (D₂O, 150 MHz) δ 149.7, 148.1 (d, J_{F-C}=240.7 Hz), 148.0 (d, J_{F-C}=242.7 Hz), 132.9 (2×), 102.2 (d, J_{F-C}=6.7 Hz), 102.1 (d, J_{F-C}=6.8 Hz), 70.9, 70.0, 69.3, 62.8, 51.2; ¹⁹F NMR (D₂O, 470 MHz) δ −143.6; MS (MALDI-TOF) calcd for C₁₂H₁₅F₂N₃O₄: 203.103.

### (1S,2R,3R,4R)-1-(5,6-difluoro-1H-benzo[d]imidazole-2-yl)pentane-1,2,3,4,5-pentaol

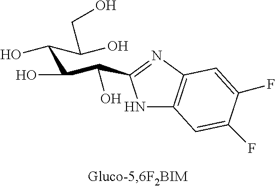

D-/L-glucose (10 mg) and 4,5-difluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GlcF₂BIM. The supporting data is given below.

C₁₂H₁₄F₂N₂O₅; black powder; [α]²⁵_{D}+5.2 (c 0.025, DMSO); ¹H NMR (MeOH-d4, 600 MHz) δ 7.47 (2H, td, J=8.6 Hz, ArH), 5.12 (1H, d, J=5.6 Hz, H1), 4.22 (1H, d, J=5.6 Hz, H2), 3.75 (1H, dd, J=11.2, 3.4 Hz, H5a), 3.70 (1H, ddd, J=8.5, 5.6, 3.8 Hz, H4), 3.61 (1H, dd, J=8.3, 1.3 Hz, H3), 3.59 (1H, dd, J=11.2, 5.7 Hz, H5b); ¹³C NMR (D₂O, 150 MHz) δ 159.1, 149.7 (d, J_{F-C}=241.7 Hz), 149.6 (d, J_{F-C}241.9 Hz), 133.1 (2×), 103.6 (d, J_{F-C}=6.7 Hz), 103.5 (d, J_{F-C}=6.8 Hz), 73.6, 73.0, 72.2, 71.1, 64.9; ¹⁹F NMR (D₂O, 470 MHz) δ −141.8; MS (MALDI-TOF) calcd for C₁₂H₁₄F₂N₂O₅: 304.087; found: m/z 304.986 [M+H]⁺.

### (1S,2R,3R)-1-(5,6-difluoro-1H-benzo[d]imidazole-2-yl)-1,2,3,4-tetrtahydroxy pentanoic acid

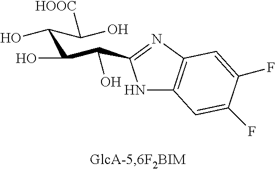

D-glucuronic acid (10 mg) and 4,5-difluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GlcAF₂BIM. The supporting data is given below.

C₁₂H₁₂F₂N₂O₆; brownish syrup; ¹H NMR (D₂O, 600 MHz) δ 7.62 (2H, t, J=8.5 Hz, ArH), 5.29 (1H, d, J=4.9 Hz, H1), 4.29-4.20 (2H, m, H2, H4), 3.90 (1H, d, J=3.0 Hz, H3); ¹³C NMR (D₂O, 150 MHz) δ 178.4, 158.9, 149.3 (d, J_{F-C}=240.6 Hz), 149.2 (d, J_{F-C}=249.2 Hz), 134.8 (2×), 103.6 (d, J_{F-C}=6.7 Hz), 103.5 (d, J_{F-C}=6.7 Hz), 76.8, 74.1, 73.2, 71.6; ¹⁹F NMR (D₂O, 470 MHz) δ −142.9; MS (MALDI-TOF) calcd for C₁₂H₁₂F₂N₂O₆: 318.066; found: m/z 318.975 [M+H]⁺.

### N-((1S,2R,3R,4R)-1-(5,6-difluoro-1H-benzo[d]imidazol-2-yl)-2,3,4,5-tetrahydroxypentyl)-acetamide

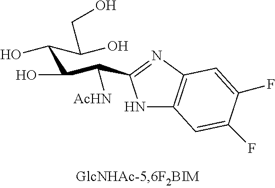

D-N-acetylglucosamine (10 mg) and 4,5-difluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GlcNAcF₂BIM. The supporting data is given below.

C₁₄H₁₇F₂N₃O₅; brownish syrup; [α]²⁵_{D}−118.6 (c 0.025, DMSO); ¹H NMR (D₂O, 600 MHz) δ 7.61 (2H, t, J=8.2 Hz, ArH), 5.45 (1H, d, J=6.2 Hz, H1), 4.45 (1H, dd, J=8.3, 6.2 Hz, H2), 3.84-3.75 (2H, m, H3, H5a), 3.61 (1H, ddd, J=8.8, 6.1, 3.6 Hz, H4), 3.44 (1H, dd, J=9.7, 8.8 Hz, H5b), 2.15 (3H, s, Me); ¹³C NMR (MeOH-d4, 150 MHz) δ 174.0, 157.0, 149.9 (d, J_{F-C}=242.5 Hz), 149.8 (d, J_{F-C}=242.1 Hz), 133.0 (d, J_{F-C}=6.3 Hz), 127.5 (d, J_{F-C}=6.5 Hz), 103.7 (d, J_{F-C}=6.2 Hz), 103.6 (d, J_{F-C}=6.8 Hz), 73.1, 72.6, 72.0, 64.8, 53.6, 22.8; ¹⁹F NMR (D₂O, 470 MHz) δ −141.2; MS (MALDI-TOF) calcd for C₁₄H₁₇F₂N₃O₅: 345.114; found: m/z 346.022 [M+H]⁺.

### (2R,3R,4R,5S)-5-amino-5-(5,6-difluoro-1H-benzo[d]imidazol-2-yl)pentane-1,2,3,4-tetraol

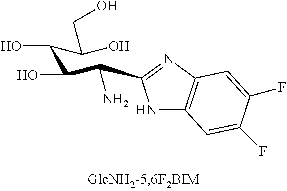

D-glucosamine (10 mg) and 4,5-difluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GlcNH₂F₂BIM. The supporting data is given below.

C₁₂H₁₅F₂N₃O₄; brownish powder; ¹H NMR (D₂O, 600 MHz) δ 7.59 (2H, td, J=8.6 Hz, ArH), 4.92 (1H, d, J=8.1 Hz, H5), 4.58 (1H, d, J=8.1 Hz, H4), 3.81-3.75 (2H, m, H2, H1a), 3.57 (1H, dd, J=11.8, 5.7 Hz, H1b), 3.29 (1H, d, J=9.5 Hz, H3); ¹³C NMR (D₂O, 150 MHz) δ 148.4, 148.3 (d, J_{F-C}=240.8 Hz), 148.2 (d, J_{F-C}=241.2 Hz), 133.0 (2×), 103.1 (d, J_{F-C}=6.7 Hz), 102.9 (d, J_{F-C}=6.8 Hz), 70.4, 70.0, 69.4, 62.7, 51.5; ¹⁹F NMR (D₂O, 470 MHz) δ −144.7; MS (MALDI-TOF) calcd for C₁₂H₁₅F₂N₃O₄: 203.103.

### (1R,2R,3R,4R)-1-(5,6-difluoro-1H-benzo[d]imidazole-2-yl)pentane-1,2,3,4,5-pentaol

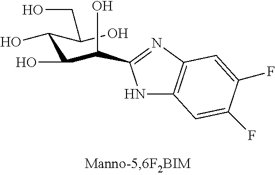

D-/L-mannose (10 mg) and 4,5-difluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give ManF₂BIM. The supporting data is given below.

C₁₂H₁₄F₂N₂O₅; brownish powder; [α]²⁵_{D}−48.6 (c 0.025, DMSO); ¹H NMR (D₂O, 600 MHz) δ 7.73 (2H, td, J=8.2 Hz, ArH), 5.23 (1H, d, J=8.8 Hz, H 1), 4.16 (1H, d, J=8.2 Hz, H2), 3.93 (1H, d, J=9.0 Hz, H3), 3.89 (1H, dd, J=11.8, 2.8 Hz, H5a), 3.80 (1H, ddd, J=8.8, 6.1, 2.8 Hz, H4), 3.72 (1H, dd, J=11.8, 6.1 Hz, H5b); ¹³C NMR (D₂O, 150 MHz) δ 155.8, 149.6 (d, J_{F-C}=246.3 Hz), 149.5 (d, J_{F-C}=246.1 Hz), 126.7 (d, J_{F-C}=6.7 Hz), 126.6 (d, J_{F-C}=6.5 Hz), 102.5 (d, J_{F-C}=6.8 Hz), 102.4 (d, J_{F-C}=7.0 Hz), 70.9, 70.5, 68.8, 66.2, 63.1; ¹⁹F NMR (D₂O, 470 MHz) δ −138.9; MS (MALDI-TOF) calcd for C₁₂H₁₄F₂N₂O₅: 304.087; found: m/z 304.984 [M+H]⁺.

### N-((1R,2R,3R,4R)-1-(5,6-difluoro-1H-benzo[d]imidazol-2-yl)-2,3,4,5-tetrahydroxypentyl)-acetamide

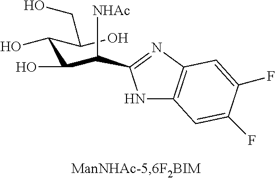

D-N-acetylmannosamine (10 mg) and 4,5-difluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give ManNAcF₂BIM. The supporting data is given below.

C₁₄H₁₇F₂N₃O₅; black powder; ¹H NMR (D₂O, 600 MHz) δ 7.64 (2H, t, J=8.4 Hz, ArH), 5.41 (1H, d, J=8.3 Hz, H1), 4.44 (1H, d, J=8.3 Hz, H2), 3.86 (1H, dd, J=11.9, 2.6 Hz, H5a), 3.79 (1H, ddd, J=6.4, 3.5, 2.6 Hz, H4), 3.76-3.65 (2H, m, H3, H5b), 2.11 (3H, s, Me); ¹³C NMR (MeOH-d4, 150 MHz) δ 174.4, 153.6, 150.3 (d, J_{F-C}=149.0 Hz), 150.2 (d, J_{F-C}=149.1 Hz), 128.9 (d, J_{F-C}=6.3 Hz), 127.5 (d, J_{F-C}=6.5 Hz), 102.5 (d, J_{F-C}=6.7 Hz), 102.4 (d, J_{F-C}=6.5 Hz), 70.5, 69.4, 69.3, 62.9, 49.7, 21.7; ¹⁹F NMR (D₂O, 470 MHz) δ −141.2; MS (MALDI-TOF) calcd for C₁₄H₁₇F₂N₃O₅: 345.114; found: m/z 346.033 [M+H]⁺.

### (1S,2R,3R,4R)-1-(5,6-difluoro-1H-benzo[d]imidazol-2-yl)-3-O-(2′,3′,4′,5′-tetrahydroxy-α-D-glucopyranosyl)pentane-1,2,4,5-tetraol

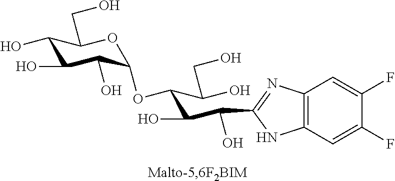

Maltose (10 mg) and 4,5-difluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give MaltoF₂BIM. The supporting data is given below.

C₁₈H₂₄F₂N₂O₁₀; black syrup; ¹H NMR (D₂O, 600 MHz) δ 7.63 (2H, t, J=8.3 Hz, ArH), 5.43 (1H, d, J=3.3 Hz, H1), 5.17 (1H, d, J=3.8 Hz, H1′), 4.37 (1H, dd, J=5.1, 3.5 Hz, H2), 4.08 (1H, ddd, J=8.6, 5.0, 4.1 Hz, H4), 3.97 (1H, t, J=5.0 Hz, H3), 3.89 (1H, ddd, J=9.0, 4.6, 2.0 Hz, H5′), 3.87-3.83 (2H, m, H2′, H4′), 3.80 (1H, dd, J=12.2, 4.9 Hz, H5a), 3.75 (1H, dd, J=12.2, 5.2 Hz, H5b), 3.71 (1H, t, J=9.5 Hz, H3′), 3.61 (1H, dd, J=9.8, 3.8 Hz, H6a′), 3.45 (1H, t, J=9.8 Hz, H6b′); ¹³C NMR (D₂O, 150 MHz) δ 155.4, 148.9 (d, J_{F-C}=243.9 Hz), 148.8 (d, J_{F-C}=244.2 Hz), 128.8 (2×), 102.5 (d, J_{F-C}=6.7 Hz), 102.4 (d, J_{F-C}=6.7 Hz), 100.4, 80.6, 73.0, 72.8, 72.5, 72.4, 71.5, 69.3, 67.8, 62.2, 60.3; ¹⁹F NMR (D₂O, 470 MHz) δ −141.4; MS (MALDI-TOF) calcd for C₁₈H₂₄F₂N₂O₁₀: 466.140; found: m/z 367.098 [M+H]⁺.

### Maltotrio-5,6-F₂BIM

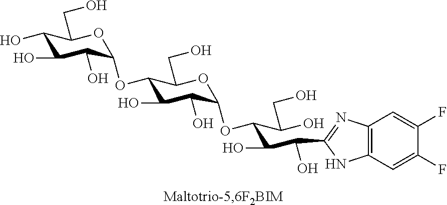

Maltotriose (10 mg) and 4,5-difluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give MaltotrioF₂BIM. The supporting data is given below.

C₂₄H₃₄F₂N₂O₁₅; brownish powder; ¹H NMR (D₂O, 600 MHz) δ 7.62 (2H, t, J=8.3 Hz, ArH), 5.46 (1H, d, J=3.2 Hz, H1), 5.38 (1H, d, J=3.8 Hz, H1′), 5.17 (1H, d, J=3.8 Hz, H1″), 4.37 (1H, dd, J=5.0, 3.3 Hz, H2), 4.09 (1H, ddd, J=6.8, 4.0, 3.4 Hz, H4), 4.00-3.98 (2H, m), 3.91-3.57 (12H, m), 3.42 (1H, t, J=9.5 Hz; ¹³C NMR (D₂O, 150 MHz) δ 155.2, 149.1 (d, J_{F-C}=245.1 Hz), 149.0 (d, J _{F-C}=244.9 Hz), 127.9 (2×), 102.5 (d, J_{F-C}=6.8 Hz), 102.4 (d, J_{F-C}=6.7 Hz), 100.1, 99.8, 80.6, 76.8, 73.2, 72.9, 72.8, 72.6, 72.3, 71.7, 71.2, 71.0, 69.3, 67.6, 62.1, 60.4, 60.3; ¹⁹F NMR (D₂O, 470 MHz) δ −140.3; MS (MALDI-TOF) calcd for C₂₄H₃₄F₂N₂O₁₅: 628.193.

### Maltohexaose-5,6F₂BIM

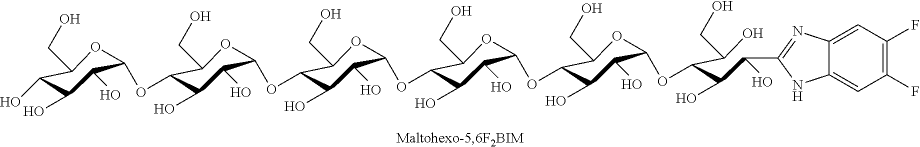

Maltohexose (10 mg) and 4,5-difluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give MaltohexoF₂BIM. The supporting data is given below.

C₄₂H₆₄F₂N₂O₃₀; ¹H NMR (D₂O, 600 MHz) δ 7.67 (2H, t, J=8.3 Hz, ArH), 5.44 (1H, d, J=3.4 Hz, H1′), 5.41 (3H, s, H1″, H1′″, H1″″), 5.38 (1H, d, J=3.7 Hz, H1″″40 ), 5.17 (1H, d, J=3.5 Hz, H1), 4.39 (1H, t, J=4.2 Hz, H2), 4.09 (1H, t, J=3.4 Hz), 4.00-3.44 (33H, m); ¹³C NMR (D₂O, 150 MHz) δ 155.4, 149.0 (d, J_{F-C}=244.1 Hz), 149.0 (d, J_{F-C}=244.0 Hz), 128.6 92×), 102.5 (d, J_{F-C}=6.7 Hz), 102.4 (d, J_{F-C}=6.7 Hz), 100.1, 99.7, 99.6, 99.5 (2×), 80.6, 77.0 (2×), 76.9, 76.8, 76.7, 73.3 (2×), 73.2, 73.1, 73.0, 72.8, 72.7, 72.4, 71.7, 71.5 (3×), 71.2, 71.1 (2×), 70.9, 69.3, 67.7, 62.1, 60.4 (2×), 60.3 (2×), 60.2; ¹⁹F NMR (D₂O, 470 MHz) δ −140.0; MS (MALDI-TOF) calcd for C₄₂H₆₄F₂N₂O₃₀: 1114.351.

### (1R,2R,3R,4R)-1-(5,6-difluoro-1H-benzo[d]imidazole-2-yl)pentane-1,2,3,4-tetraol

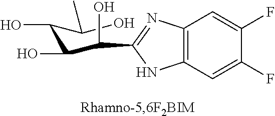

D-/L-rhamnose (10 mg) and 4,5-difluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give RhaF₂BIM. The supporting data is given below.

C₁₂H₁₄F₂N₂O₄; brownish syrup; ¹H NMR (D₂O, 600 MHz) δ 7.71 (2H, t, J=8.0 Hz, ArH), 5.20 (1H, d, J=8.1 Hz, H1), 4.21 (1H, d, J=8.1 Hz, H2), 3.93 (1H, dd, J=8.2, 5.9 Hz, H4), 3.72 (1H, d, J=8.2 Hz, H3), 1.33 (3H, d, J=5.9 Hz, Me); ¹³C NMR (MeOH-d4, 150 MHz) δ 160.0, 149.6 (d, J_{F-C}=241.1 Hz), 149.7 (d, J_{F-C}=240.8 Hz), 133.8 (d, J_{F-C}=6.3 Hz), 127.5 (d, J_{F-C}=6.5 Hz), 103.5 (d, J_{F-C}=6.2 Hz), 103.4 (d, J_{F-C}=6.8 Hz), 75.2, 73.2, 70.2, 68.7, 20.7; ¹⁹F NMR (D₂O, 470 MHz) δ −139.5; MS (MALDI-TOF) calcd for C₁₂H₁₄F₂N₂O₄: 288.092; found: m/z 288.998 [M+H]⁺.

### (1S,2S,3R)-1-(5,6-difluoro-1H-benzo[d]imidazole-2-yl)butane-1,2,3,4-tetrad

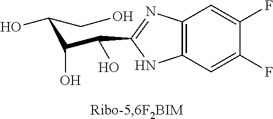

D-/L-ribose (10 mg) and 4,5-difluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give RibF₂BIM. The supporting data is given below.

C₁₁H₁₂F₂N₂O₄; brownish syrup; ¹H NMR (D₂O, 600 MHz) δ 7.74 (2H, t, J=7.7 Hz, ArH), 5.47 (1H, s, H1), 4.15 (1H, s, H2), 3.82 (1H, d, J=8.8 Hz, H3), 3.73 (2H, d, J=6.4 Hz, H4a, H4b); ¹³C NMR (MeOH-d4, 150 MHz) δ 158.3, 150.3 (d, J_{F-C}=245.9 Hz), 150.2 (d, J_{F-C}=246.8 Hz), 129.5 (2×), 103.7 (d, J_{F-C}=6.7 Hz), 103.6 (d, J_{F-C}=6.5 Hz), 75.2, 73.5, 69.7, 64.5; ¹⁹F NMR (D₂O, 470 MHz) δ −139.1; MS (MALDI-TOF) calcd for C₁₁H₁₂F₂N₂O₄: 274.077; found: m/z 274.932 [M+H]⁺.

### (1S,2R,3R)-1-(5,6-difluoro-1H-benzo[d]imidazole-2-yl)butane-1,2,3,4-tetraol

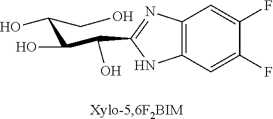

D-/L-xylose (10 mg) and 4,5-difluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give XylF₂BIM. The supporting data is given below.

C₁₁H₁₂F₂N₂O₄; black syrup; ¹H NMR (MeOH-d4, 600 MHz) δ 7.64 (2H, t, J=8.2 Hz, ArH), 5.25 (1H, d, J=4.7 Hz, H1), 4.11 (1H, dd, J=4.7, 1.8 Hz, H2), 3.87 (1H, dd,=5.6, 1.8 Hz, H3), 3.66 (1H, dd, J=10.9, 6.2 Hz, H4a), 3.61 (1H, dd, J=10.9, 6.1 Hz, H4b); ¹³C NMR (MeOH-d4, 150 MHz) δ 159.2, 150.6 (d, J_{F-C}=245.4 Hz), 150.5 (d, J_{F-C}=245.7 Hz), 129.5 (2×), 103.6 (d, J_{F-C}=6.0 Hz), 103.5 (d, J_{F-C}=6.8 Hz), 73.7, 71.4, 69.2, 64.2; ¹⁹F NMR (D₂O, 470 MHz) δ −139.3; MS (MALDI-TOF) calcd for C₁₁H₁₂F₂N₂O₄: 274.077; found: m/z 274.890 [M+H]⁺.

### N-((2R,3R,4R,5R,6R)-1-(6,7-difluoro-3-oxo-3,4-dihydroquinoxalin-2-yl)-2,4,5,6,7-pentahydroxyheptan-3-yl)acetamide

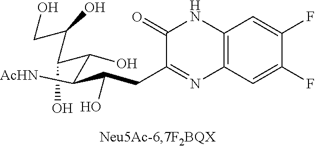 [SMILES: CC(=O)N[C@@H](C(O)[C@H](O)[C@H](O)CO)[C@H](O)Cc1nc2cc(F)c(F)cc2[nH]c1=O]

Sialic acid (Neu5Ac; 10 mg) and 4,5-difluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give Neu5AcF₂BIM (SiaF₂BIM). The supporting data is given below.

C₁₇H₂₁F₂N₃O₇; brownish powder; [α]²⁵_{D}−75.3 (c 0.025, DMSO); ¹H NMR (DMSO-d6, 600 MHz) δ 7.82 (1H, dd, J=10.9, 8.3 Hz, ArH), 7.20 (1H, dd, J=10.9, 7.8 Hz, ArH), 3.74 (1H, d, J=11.7 Hz, H7a), 3.72 (1H, d, J=11.7 Hz, H7b), 3.59 (1H, dd, J=11.0, 3.2 Hz, H3), 3.49 (1H, ddd, J=9.2, 4.8, 3.3 Hz, H2), 3.40-3.30 (2H, m, H4, H6), 3.19 (1H, d, J=8.5 Hz, H5), 2.92 (1H, dd, J=14.2, 9.2 Hz, H1a), 2.76 (1H, dd, J=14.2, 4.5 Hz, H1b), 1.95 (3H, s, Me); ¹³C NMR (DMSO-d6, 150 MHz) δ 171.2, 160.9, 154.5 (d, J_{F-C}=17.9 Hz), 149.8 (dd, J_{F-C}=247.9, 14.5 Hz), 145.5 (dd, J_{F-C}=240.9, 15.0 Hz), 129.1 (d, J_{F-C}=9.9 Hz), 128.1 (d, J_{F-C}=10.1 Hz), 115.9 (d, J_{F-C}=18.0 Hz), 102.9 (d, J_{F-C}=21.3 Hz), 70.9, 70.1, 67.9, 65.9, 63.8, 54.1, 38.4, 22.5; ¹⁹F NMR (D₂O, 470 MHz) δ −132.4; HRMS (ESI) calcd for C₁₇H₂₁F₂N₃O₇: 417.135; found: m/z 440.062 [M+Na]⁺.

### N-((2R,3R,4R,5R,6R)-1-(6,7-difluoro-3-oxo-3,4-dihydroquinoxalin-2-yl)-2,4,5,6,7-pentahydroxyheptan-3-yl)-2-hydroxyacetamide

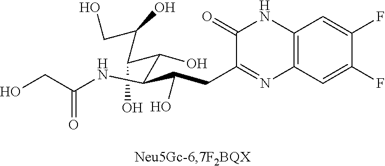 [SMILES: O=C(CO)N[C@@H](C(O)[C@H](O)[C@H](O)CO)[C@H](O)Cc1nc2cc(F)c(F)cc2[nH]c1=O]

Neu5Gc (10 mg) and 4,5-difluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give Neu5GcF₂BIM. The supporting data is given below.

C₁₇H₂₁F₂N₃O₈; black powder; ¹H NMR (D₂O, 600 MHz) δ 7.70 (1H, dd, J=10.0, 8.2 Hz, ArH), 7.28 (1H, dd, J=10.0, 7.4 Hz, ArH), 4.17 (2H, s, CH₂), 4.05 (1H, dd, J=10.0, 4.0 Hz, H2), 4.03 (1H, d, J=10.0 Hz, H5), 4.00 (1H, dd, J=9.5 Hz, H4), 3.85 (1H, dd, J=11.8, 2.4 Hz, H7a), 3.77 (1H, ddd, J=10.0, 6.5, 2.4 Hz, H6), 3.63 (1H, dd, J=11.8, 6.5 Hz, H7b), 3.49 (1H, t, J=9.6 Hz, H3), 3.08-3.02 (2H, m, H1a, H1b); ¹³C NMR (D₂O, 150 MHz) δ 174.6, 156.2, 152.5 (d, J_{F-C}=17.8 Hz), 149.8 (dd, J_{F-C}=247.9, 14.5 Hz), 145.5 (dd, J_{F-C}=240.9, 15.0 Hz), 129.1 (d, J_{F-C}=9.9 Hz), 126.2 (d, J_{F-C}=10.1 Hz), 115.3 (d, J_{F-C}=18.0 Hz), 102.2 (d, J_{F-C}=21.3 Hz), 70.6, 69.3, 69.0, 67.7, 66.4, 60.9, 52.9, 37.5; ¹⁹F NMR (D₂O, 470 MHz) δ −133.0; HRMS (ESI) calcd for C₁₇H₂₁F₂N₃O₈: 4133.130.

### 6,7-difluoro-3-((2R,3R,4R,5R,6R)-2,3,4,5,6,7-hexahydroxyheptyl)quinoxalin-2(1H)-one

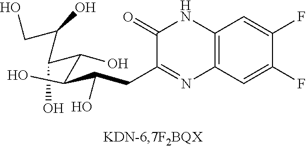 [SMILES: O=c1[nH]c2cc(F)c(F)cc2nc1C[C@@H](O)[C@@H](O)C(O)[C@H](O)[C@H](O)CO]

KDN (10 mg) and 4,5-difluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give KDNF₂BIM. The supporting data is given below.

C₁₅H₁₈F₂N₂O₇; brownish powder; ¹H NMR (D₂O, 600 MHz) δ 7.74 (1H, dd, J=10.6, 6.6 Hz, ArH), 7.28 (1H, dd, J=10.6, 7.3 Hz, ArH), 4.55 (1H, dd, J=8.6, 4.2 Hz, H2), 3.96 (1H, dd, J=9.5, 4.1 Hz, H4), 3.88 (1H, dd, J=11.8, 2.6 Hz, H7a), 3.85 (1H, d, J=8.9 Hz, H3), 3.79 (1H, ddd, J=9.0, 6.2, 2.6 Hz, H6), 3.69 (1H, dd, J=11.8, 6.1 Hz, H7b), 3.60 (1H, t, J=9.0 Hz, H5), 3.23 (1H, dd, J=14.2, 5.5 Hz, H1a), 3.18 (1H, dd, J=14.2, 4.6 Hz, H1b); ¹³C NMR (DMSO-d6, 150 MHz) δ 161.6, 154.7, 149.9 (dd, J_{F-C}=247.8, 14.9 Hz), 149.8 (dd, J_{F-C}=247.9, 14.0 Hz). 129.1 (d, J_{F-C}=9.8 Hz), 128.1 (d, J_{F-C}=8.9 Hz), 115.9 (d, J_{F-C}=17.9 Hz), 102.9 (d, J_{F-C}=21.8 Hz), 71.7, 71.5, 69.7, 68.8, 67.9, 63.9, 38.1; ¹⁹F NMR (D₂O, 470 MHz) δ −136.8; HRMS (ESI) calcd for C₁₅H₁₈F₂N₂O₇: 376.108.

### 6,7-difluoro-3-((2R,3R,4R,5R)-2,3,4,5,6-pentahydroxyheptyl)quinoxalin-2(1H)-one

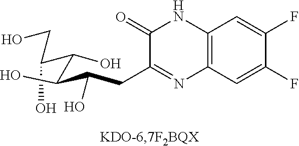 [SMILES: O=c1[nH]c2cc(F)c(F)cc2nc1C[C@@H](O)[C@@H](O)C(O)[C@H](O)CO]

KDO (10 mg) and 4,5-difluorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give KDOF₂BIM. The supporting data is given below.

C₁₄H₁₆F₂N₂O₆; brownish powder; [α]²⁵_{D}+30.3 (c 0.010, DMSO); ¹H NMR (D₂O, 600 MHz) δ 7.66 (1H, dd, J=10.5, 7.9 Hz, ArH), 7.24 (1H, dd, J=10.5, 7.3 Hz, ArH), 4.30 (1H, td, J=9.0, 3.4 Hz, H2), 3.88 (1H, dd, J=11.8, 3.0 Hz, H6a), 3.87 (1H, d, J=8.9 Hz, H4), 3.83 (1H, d, J=8.4 Hz, H3), 3.79 (1H, ddd, J=8.9, 6.4, 3.0 Hz, H5), 3.69 (1H, dd, J=11.8, 6.4 Hz, H6b), 3.39 (1H, dd, J=14.6, 3.4 Hz, H1a), 2.99 (1H, dd, J=14.6, 9.2 Hz, H1b); ¹³C NMR (D₂O, 150 MHz) δ 159.6, 156.2, 151.3 (dd, J_{F-C}=250.5, 14.9 Hz), 147.2 (dd, J_{F-C}=243.3, 14.0 Hz), 128.3 (d, J_{F-C}=9.5 Hz), 127.8 (d, J_{F-C}=10.0 Hz), 115.2 (d, J_{F-C}=18.7 Hz), 103.8 (d, J_{F-C}=22.2 Hz), 72.3, 70.9, 69.2, 68.9, 63.1, 37.5; ¹⁹F NMR (D₂O, 470 MHz) δ −133.4; HRMS (ESI) calcd for C₁₄H₁₆F₂N₂O₆: 346.098.

Sugar-5CF₃BIMs

### (1R,2S,3R)-1-(5-(trifluoromethyl)-1H-benzo[d]imidazol-2-yl)butane-1,2,3,4-tetraol

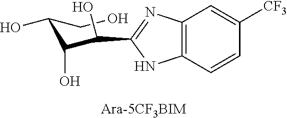

D-/L-arabinose (10 mg) and 4-trifluoromethanephenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give AraCF₃BIM. The supporting data is given below.

C₁₂H₁₃F₃N₂O₄; pale yellow powder; ¹H NMR (D₂O, 600 MHz) δ 8.07 (1H, s, ArH), 7.83 (1H, d, J=8.6 Hz, ArH), 7.72 (1H, d, J=8.3 Hz, ArH), 5.44 (1H, s, H1), 4.06-3.73 (4H, m, H2, H3, H4a, H4b); ¹³C NMR (DMSO-d6, 150 MHz) δ 160.7, 127.9, 126.1, 124.3, 122.5 (d, J_{F-C}=3.4 Hz), 121.9 (q, J_{F-C}=31.1 Hz), 117.8, 115.0 (d, J_{F-C}=4.2 Hz), 73.8, 70.9, 67.6, 63.5; ¹⁹F NMR (D₂O, 470 MHz) δ −61.76; MS (MALDI-TOF) calcd for C₁₂H₁₃F₃N₂O₄: 306.083; found: m/z 307.133 [M+H]⁺.

### (1S,2R,3S,4R)-1-(5-(trifluoromethyl)-1H-benzo[d]imidazol-2-yl)pentane-1,2,3,4-tetraol

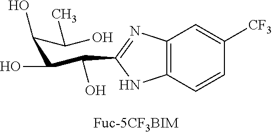

D-/L-fucose (10 mg) and 4-trifluoromethanephenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give FucCF₃BIM. The supporting data is given below.

C₁₃H₁₅F₃N₂O₄; white powder; ¹H NMR (DMSO-d6, 600 MHz) δ 7.82 (1H, s, ArH), 7.66 (1H, s, ArH), 7.44 (1H, d, J=8.1 Hz, ArH), 5.62 (1H, d, J=6.2 Hz, H1), 5.18 (1H, d, J=5.4 Hz, OH), 4.55 (1H, d, J=7.4 Hz, OH), 4.41 (1H, d, J=7.7 Hz, OH), 4.20 (1H, d, J=6.2 Hz, OH), 3.92 (1H, dd, J=7.5, 6.3 Hz, H2), 3.90 (1H, m, H4), 3.38 (1H, t, J=7.5 Hz, H3), 1.14 (3H, d, J=7.5 Hz, CH₃); ¹³C NMR (DMSO-d6, 150 MHz) δ 160.9, 128.0, 126.2, 124.4, 122.6, 121.7 (q, J_{F-C}=31.0 Hz), 117.8, 112.3, 73.3, 72.5, 67.8, 65.1, 20.1; ¹⁹F NMR (D₂O, 470 MHz) δ −62.11; MS (MALDI-TOF) calcd for C₁₃H₁₅F₃N₂O₄: 320.098; found: m/z 321.132 [M+H]⁺.

### (1S,2R,3S,4R)-1-(5-(trifluoromethyl)-1H-benzo[d]imidazol-2-yl)pentane-1,2,3,4,5-pentaol

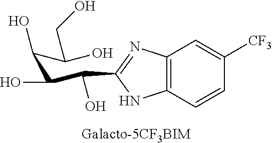

D-/L-galactose (10 mg) and 4-trifluoromethanephenyldiamine (10 mg) with catalytic amount of iodine (1 mg) in acetic acid (1 mL) was mixed at room temperature for 12 hour. The resulting solution was precipitated by ethyl acetate (30 mL) and centrifuged for three times to remove the excess reagent. The pellet was lyophilized to give GalCF₃BIM. The supporting data is given below.

C₁₃H₁₅F₃N₂O₅; white powder; ¹H NMR (D₂O, 600 MHz) δ 8.15 (1H, s, ArH), 7.91 (1H, d, J=8.7 Hz, ArH), 7.85 (1H, d, J=8.6 Hz, ArH), 5.64 (1H, d, J=1.0 Hz, H1), 4.17 (1H, dd, J=9.5, 1.1 Hz, H2), 4.02 (1H, ddd, J=9.0, 6.3, 5.4 Hz, H4), 3.91 (1H, d, J=9.0 Hz, H3), 3.75-3.72 (2H, m, H5a, H5b); ¹³C NMR (D₂O, 150 MHz) δ 157.7, 134.0, 131.7, 127.1 (q, J_{F-C} =32.2 Hz), 124.9, 122.5, 114.8, 112.2, 72.4, 69.8, 68.9, 66.8, 63.0; ¹⁹F NMR (D₂O, 470 MHz) δ −61.86; MS (MALDI-TOF) calcd for C₁₃H₁₅F₃N₂O₅: 336.093; found: m/z 337.135 [M+H]⁺.

### (1S,2R,3S)-1-(5-(trifluoromethyl)-1H-benzo[d]imidazole-2-yl)-1,2,3,4-tetrtahydroxy pentanoic acid

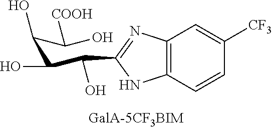

D-galacturonic acid (10 mg) and 4-trifluoromethanephenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GalACF₃BIM. The supporting data is given below.

C₁₃K₃F₃N₂O₆; white powder; ¹H NMR (D₂O, 600 MHz) δ 8.17 (1H, s, ArH), 7.94 (1H, d, J=8.8 Hz, ArH), 7.88 (1H, d, J=8.8 Hz, ArH), 5.66 (1H, d, J=1.6 Hz, H1), 4.51 (1H, s, H4), 4.28 (1H, dd, J=9.5, 1.0 Hz, H3), 4.16 (1H, dd, J=9.5, 1.6 Hz, H2); ¹³C NMR (D₂O, 150 MHz) δ 177.3, 157.5, 133.5, 131.0, 127.4 (q, J_{F-C}=33.0 Hz), 124.8, 122.8, 114.8, 112.2, 72.4, 70.8, 71.5, 66.5; ¹⁹F NMR (D₂O, 470 MHz) δ −62.16; MS (MALDI-TOF) calcd for C₁₃H₁₃F₃N₂O₆: 350.073; found: m/z 351.123 [M+H]⁺.

### N-((1S,2R,3S,4R)-1-(5-(trifluoromethyl)-1H-benzo[d]imidazol-2-yl)-2,3,4,5-tetrahydroxypentyl)-acetamide

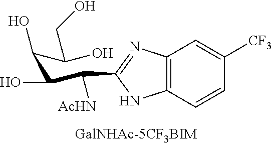

D-N-acetylgalactosamine (10 mg) and 4-trifluoromethanephenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GalNAcCF₃BIM. The supporting data is given below.

C₁₅H₁₈F₃N₂O₅; pale brownish powder; ¹H NMR (D₂O, 600 MHz) δ 8.11 (1H, s, ArH), 7.86 (1H, d, J=8.6 Hz, ArH), 7.79 (1H, d, J=8.0 Hz, ArH), 5.77 (1 H, d, J=2.0 Hz, H1), 4.40 (1H, dd, J=2.6, 2.0 Hz, H2), 4.25 (1H, dd, J=4.6, 2.6 Hz, H3), 3.85-3.65 (3H, m, H4, H5a, H5b), 2.07 (3H, s, Me); ¹³C NMR (D₂O, 150 MHz) δ 175.6, 155.2, 135.1, 132.8, 127.0 (q, J_{F-C}=32.0 Hz), 122.0, 114.9, 112.4, 82.6, 80.1, 74.9, 70.9, 60.1, 22.1; ¹⁹F NMR (D₂O, 470 MHz) δ −61.74; MS (MALDI-TOF) calcd for C₁₅H₁₈F₃N₂O₅: 377.120; found: m/z 378.195 [M+H]⁺.

### (1S,2R,3R,4R)-1-(5-(trifluoromethyl)-1H-benzo[d]imidazol-2-yl)pentane-1,2,3,4,5-pentaol

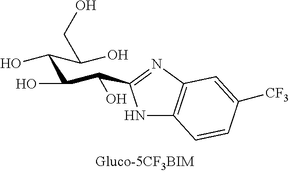

D-/L-glucose (10 mg) and 4-trifluoromethanephenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GlcCF₃BIM. The supporting data is given below.

C₁₃H₁₅F₃N₂O₅; yellow powder; ¹H NMR (D₂O, 600 MHz) δ 8.15 (1H, s, ArH), 7.92 (1H, d, J=8.6 Hz, ArH), 7.88 (1H, d, J=8.7 Hz, ArH), 5.51 (1H, d, J=5.0 Hz, H1), 4.45 (1H, d, J=5.1 Hz, H2), 3.84 (1H, d, J=8.8 Hz, H3), 3.81-3.77 (2H, m, H4, H5a), 3.62 (1H, dd, J=12.4, 6.6 Hz, H5b); ¹³C NMR (D₂O, 150 MHz) δ 156.8, 132.7, 130.2, 127.5 (q, J_{F-C}=33.0 Hz), 124.7, 122.9 (d, J_{F-C}=3.4 Hz), 114.7, 111.9 (d, J_{F-C}=4.2 Hz), 70.7, 70.6, 68.9, 67.3, 62.7; ¹⁹F NMR (D₂O, 470 MHz) δ −62.24; MS (MALDI-TOF) calcd for C₁₃H₁₅F₃N₂O₅: 336.093; found: m/z 337.135 [M+H]⁺.

### (1S,2R,3R)-1-(5-(trifluoromethyl)-1H-benzo[d]imidazole-2-yl)-1,2,3,4-tetrtahydroxy pentanoic acid

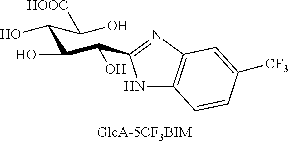

D-glucuronic acid (10 mg) and 4-trifluoromethanephenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GlcACF₃BIM. The supporting data is given below.

C₁₃H₁₃F₃N₂O₆; yellow powder; ¹H NMR (D₂O, 600 MHz) δ 8.09 (1H, s, ArH), 7.86 (1H, d, J=8.9 Hz, ArH), 7.80 (1H, d, J=8.3 Hz, ArH), 5.43 (1H, s, H1), 4.33 (1H, s, H2), 3.22 (1H, s, H4), 4.02 (1H, s, H3); ¹³C NMR (D₂O, 150 MHz) δ 177.7, 156.6, 134.3, 132.0, 126.8 (q, J_{F-C}=29.7 Hz), 123.2, 122.0, 114.8, 112.2 (d, J_{F-C}=4.0 Hz), 73.1, 72.2, 71.0, 67.6; ¹⁹F NMR (D₂O, 470 MHz) δ −63.50; MS (MALDI-TOF) calcd for C13H13F3N2O6: 350.073; found: m/z 351.116 [M+H]+.

### N-((1S,2R,3R,4R)-1-(5-(trifluoromethyl)-1H-benzo[d]imidazol-2-yl)-2,3,4,5-tetrahydroxypentyl)-acetamide

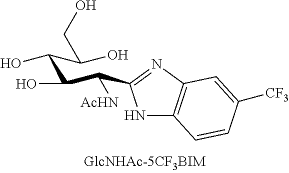

D-N-acetylglucosamine (10 mg) and 4-trifluoromethanephenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GlcNAcCF₃BIM. The supporting data is given below.

C₁₅H₁₈F₃N₂O₅; yellow powder; ¹H NMR (D₂O, 600 MHz) δ 8.07 (1H, s, ArH), 7.84 (1H, d, J=8.6 Hz, ArH), 7.77 (1H, d, J=7.9 Hz, ArH), 5.68 (1H, s, H1), 4.50 (1H, m, H2), 4.19-3.54 (4H, m, H3, H4, H5a, H5b), 2.18 (3H, s, Me); ¹³C NMR (D₂O, 150 MHz) δ 174.9, 154.1, 134.4, 132.1, 126.9 (q, J_{F-C}=32.4 Hz), 123.1, 122.3 (d, J_{F-C}=3.4 Hz), 114.8, 112.3 (d, J_{F-C}=4.4 Hz), 82.4, 75.7, 74.9, 70.8, 61.4, 22.0; ¹⁹F NMR (D₂O, 470 MHz) δ −62.32; MS (MALDI-TOF) calcd for C₁₅H₁₈F₃N₂O₅: 377.120; found: m/z 378.183 [M+H]+.

### (1R,2R,3R,4R)-1-(5-(trifluoromethyl)-1H-benzo[d]imidazole-2-yl)pentane-1,2,3,4,5-pentaol

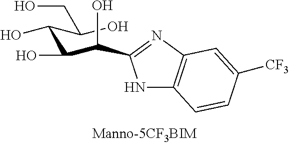

D-/L-mannose (10 mg) and 4-trifluoromethanephenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give ManCF₃BIM. The supporting data is given below.

C₁₃H₁₅F₃N₂O₅; pale brownish powder; ¹H NMR (D₂O, 600 MHz) δ 8.17 (1H, s, ArH), 7.94 (1H, d, J=8.6 Hz, ArH), 7.88 (1H, d, J=8.7 Hz, ArH), 5.28 (1 H, d, J=8.7 Hz, H1), 4.20 (1H, d, J=8.7 Hz, H2), 3.95 (1H, d, J=9.0 Hz, H3), 3.90 (1H, dd, J=11.8, 2.8 Hz, H5a), 3.81 (1H, ddd, J=9.0, 6.1, 2.8 Hz, H4), 3.72 (1H, dd, J=11.8, 6.1 Hz, H5b); ¹³C NMR (D₂O, 150 MHz) δ 156.9, 133.5, 131.1, 127.4 (q, J_{F-C}=32.5 Hz), 124.8, 122.7, 114.8, 112.2 (d, J_{F-C}=4.1 Hz), 70.9, 70.5, 68.8, 66.4, 63.1; ¹⁹F NMR (D₂O, 470 MHz) δ −61.90; MS (MALDI-TOF) calcd for C₁₃H₁₅F₃N₂O₅: 336.093; found: m/z 337.145 [M+H]⁺.

### (1R,2R,3R,4R)-1-(5-(trifluoromethyl)-1H-benzo[d]imidazole-2-yl)pentane-1,2,3,4-tetraol

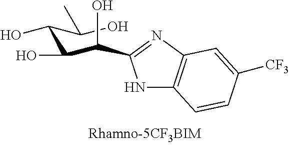

D-/L-rhamnose (10 mg) and 4-trifluoromethanephenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give RhaCF3BIM. The supporting data is given below.

C₁₃H₁₅F₃N₂O₄; white powder; ¹H NMR (D₂O, 600 MHz) δ 8.10 (1H, s, ArH), 7.85 (1H, d, J=9.1 Hz, ArH), 7.74 (1H, d, J=7.7 Hz, ArH), 5.14 (1H, d, J=7.8 Hz, H1), 4.28 (1H, d, J=7.8 Hz, H2), 3.72 (1H, d, J=7.5 Hz, H3), 3.62 (1H, dd, J=7.5, 5.5 Hz, H4), 1.32 (3H, d, J=5.4 Hz, Me); ¹³C NMR (D₂O, 150 MHz) δ 157.4, 134.0, 132.2, 126.5 (q, J_{F-C}=28.7 Hz), 123.2, 122.2, 114.8, 112.5 (d, J_{F-C}=4.0 Hz), 73.2, 72.2, 71.8, 68.0, 18.7; ¹⁹F NMR (D₂O, 470 MHz) δ −62.28; MS (MALDI-TOF) calcd for C₁₃H₁₅F₃N₂O₄: 320.098; found: m/z 321.138 [M+H]⁺.

### (1S,2S,3R)-1-(5-(trifluoromethyl)-1H-benzo[d]imidazole-2-yl)butane-1,2,3,4-tetraol

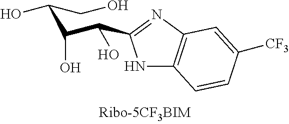

D-/L-ribose (10 mg) and 4-trifluoromethanephenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give RibCF3BIM. The supporting data is given below.

C₁₂H₁₃F₃N₂O₄; yellow powder; ¹H NMR (D₂O, 600 MHz) δ 8.17 (1H, s, ArH), 7.94 (1H, d, J=8.3 Hz, ArH), 7.88 (1H, d, J=8.4 Hz, ArH), 5.53 (1H, s, H1), 4.18 (1H, s, H2), 3.85-3.72 (3H, m, H3, H4a, H4b); ¹³C NMR (D₂O, 150 MHz) δ 155.6, 132.8, 130.3, 127.6 (q, J_{F-C}=32.7 Hz), 124.7, 123.0 (d, J_{F-C}=3.2 Hz), 114.8, 112.1 (d, J_{F-C}=4.1 Hz), 72.8, 70.9, 67.5, 62.5; ¹⁹F NMR (D₂O, 470 MHz) δ −62.27; MS (MALDI-TOF) calcd for C₁₂H₁₃F₃N₂O₄: 306.083; found: m/z 307.093 [M+H]⁺.

### (1S,2R,3R)-1-(5-(trifluoromethyl)-1H-benzo[d]imidazole-2-yl)butane-1,2,3,4-tetraol

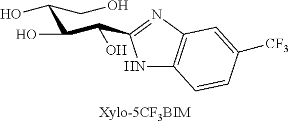

D-/L-xylose (10 mg) and 4-trifluoromethanephenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give XylCF3BIM. The supporting data is given below.

C₁₂H₁₃F₃N₂O₄; yellow powder; ¹H NMR (D₂O, 600 MHz) δ 8.07 (1H, s, ArH), 7.84 (1H, s, ArH), 7.79 (1H, s, ArH), 5.47 (1H, s, H1), 4.19 (1H, s, H2), 3.96 (1H, s, H3), 3.76 (1H, d, J=11.4 Hz, H4a), 3.70 (1H, d, J=11.4 Hz, H4b); ¹³C NMR (D₂O, 150 MHz) δ 156.6, 132.9, 130.4, 127.4 (q, J_{F-C}=32.8 Hz), 124.7, 122.8 (d, J_{F-C}=3.2 Hz), 114.7, 111.9 (d, J_{F-C}=4.1 Hz), 72.1, 70.3, 67.2, 62.4; ¹⁹F NMR (D₂O, 470 MHz) δ −62.26; MS (MALDI-TOF) calcd for C₁₂H₁₃F₃N₂O₄: 306.083; found: m/z 307.109 [M+H]⁺.

### N-((2R,3R,4R,5R,6R)-1-(6-trifluoromethyl-3-oxo-3,4-dihydroquinoxalin-2-yl)-2,4,5,6,7-pentahydroxyheptan-3-yl)acetamide

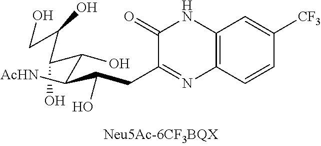 [SMILES: CC(=O)N[C@@H](C(O)[C@H](O)[C@H](O)CO)[C@H](O)Cc1nc2ccc(C(F)(F)F)cc2[nH]c1=O]

Sialic acid (Neu5Ac; 10 mg) and 4-trifluoromethanephenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give Neu5AcCF3BIM (SiaCF3BIM). The supporting data is given below.

C₁₈H₂₂F₃N₃O₇; white powder; ¹H NMR (D₂O, 600 MHz) δ 8.06 (1H, s, ArH), 7.81 (1H, d, J=8.6 Hz, ArH), 7.43 (1H, d, J=8.7 Hz, ArH), 4.80 (1H, m, H2), 4.07 (1H, d, J=10.0 Hz, H4), 4.00 (1H, d, J=10.1 Hz, H5), 3.85 (1H, dd, J=11.8, 2.5 Hz, H7a), 3.77 (1H, ddd, J=10.0, 6.3, 2.5 Hz, H6), 3.64 (1H, dd, J=11.8, 6.3 Hz, H7b), 3.49 (1H, d, J=9.1 Hz, H3), 3.06 (2H, m, H1a, H1b), 2.09 (3H, s, Me); ¹³C NMR (D₂O, 150 MHz) δ 174.4, 160.2, 156.3, 133.1, 131.0, 128.6, 126.7, 125.8 (q, J_{F-C}=33.3 Hz), 125.2, 116.7, 70.7, 69.3, 67.8, 66.7, 63.2, 53.4, 37.5, 21.9; ¹⁹F NMR (D₂O, 470 MHz) δ −62.72; HRMS (ESI) calcd for C₁₈H₂₂F₃N₃O₇: 449.141; found: m/z 432.218 [M−H₂O+H]⁺.

### N-((2R,3R,4R,5R,6R)-1-(6-trifluoromethyl-3-oxo-3,4-dihydroquinoxalin-2-yl)-2,4,5,6,7-pentahydroxyheptan-3-yl)-2-hydroxyacetamide

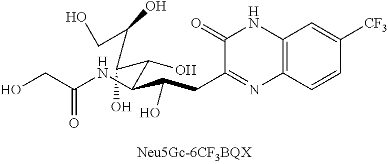 [SMILES: O=C(CO)N[C@@H](C(O)[C@H](O)[C@H](O)CO)[C@H](O)Cc1nc2ccc(C(F)(F)F)cc2[nH]c1=O]

Neu5Gc (10 mg) and 4-trifluoromethanephenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give Neu5GcCF3BQX. The supporting data is given below.

C₁₈H₂₂F₃N₃O₈; white powder; ¹H NMR (D₂O, 600 MHz) δ 8.13 (1H, s, ArH), 7.86 (1H, d, J=8.6 Hz, ArH), 7.49 (1H, d, J=8.6 Hz, ArH), 4.17 (1H, m, H2), 4.15 (2H, s, CH₂), 4.07 (1H, d, J=9.9 Hz, H5), 4.02 (1H, t, J=10.0 Hz, H4), 3.85 (1H, dd, J=11.9, 2.6 Hz, H7a), 3.77 (1H, ddd, J=10.0, 6.2, 2.6 Hz, H6), 3.63 (1H, dd, J=11.8, 6.2 Hz, H7b), 3.50 (1H, d, J=9.1 Hz, H3), 3.13-3.04 (2H,m, H1a, H1b); ¹³C NMR (D₂O, 150 MHz) δ 175.1, 160.1, 156.4, 133.2, 131.2, 128.7, 126.8, 125.9 (q, J_{F-C}=33.9 Hz), 125.3, 116.7, 70.6, 70.1, 69.3, 67.7, 66.7, 60.9, 53.0, 37.5; ¹⁹F NMR (D₂O, 470 MHz) δ −62.73; HRMS (ESI) calcd for C₁₈H₂₂F₃N₃O₈: 465.136; found: m/z 448.232 [M−H₂O+H]⁺.

### 7-trifluoromethyl-3-((2R,3R,4R,5R,6R)-2,3,4,5,6,7-hexahydroxyheptyl)quinoxalin-2(1H)-one

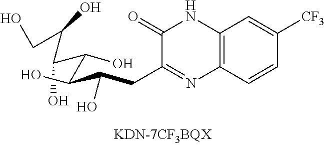 [SMILES: O=c1[nH]c2cc(C(F)(F)F)ccc2nc1C[C@@H](O)[C@@H](O)C(O)[C@H](O)[C@H](O)CO]

KDN (10 mg) and 4-trifluoromethanephenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give KDNCF3BQX. The supporting data is given below.

C₁₆H₁₉F₃N₂O₇; white powder; ¹H NMR (D₂O, 600 MHz) δ 8.19 (1H, s, ArH), 7.89 (1H, d, J=8.7 Hz, ArH), 7.55 (1H, d, J=8.6 Hz, ArH), 4.58 (1H, dd, J=8.7. 4.6 Hz, H2), 3.98 (1H, d, J=9.3 Hz, H4), 3.88 (1H, dd, J=11.8, 2.3 Hz, H7a), 3.85 (1H, d, J=8.5 Hz, H3), 3.78 (1H, ddd, J=9.0, 5.5, 2.3 Hz, H6), 3.72 (1H, d, J=9.3 Hz, H5), 3.68 (1H, dd, J=11.8, 5.5 Hz, H7b), 3.28 (1H, dd, J=14.4, 8.7 Hz, H1a), 3.20 (1H, dd, J=14.4, 4.7 Hz, H1b); ¹³C NMR (DMSO-d6, 150 MHz) δ 162.9, 156.0, 131.0, 129.3, 125.6, 125.2 (q, J_{F-C}=30.8 Hz), 123.2, 119.1, 116.5, 71.7, 71.5, 69.7, 68.8, 67.9, 64.0, 38.2; ¹⁹F NMR (D₂O, 470 MHz) δ −62.7; HRMS (ESI) calcd for C₁₆H₁₉F₃N₂O₇: 408.114; found: m/z 391.17 [M−H₂O+H]⁺.

### 7-trifluoromethyl-3-((2R,3R,4R,5R)-2,3,4,5,6-pentahydroxyheptyl)quinoxalin-2(1H)-one

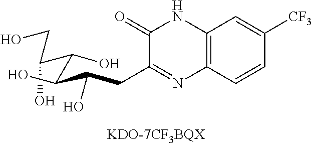 [SMILES: O=c1[nH]c2cc(C(F)(F)F)ccc2nc1C[C@@H](O)[C@@H](O)C(O)[C@H](O)CO]

KDO (10 mg) and 4-trifluoromethanephenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give KDOCF3BQX. The supporting data is given below.

C₁₅H₁₇F₃N₂O₆; white powder; ¹H NMR (D₂O, 600 MHz) δ 8.16 (1H, s, ArH), 7.87 (1H, d, J=8.6 Hz, ArH), 7.52 (1H, d, J=8.6 Hz, ArH), 4.35 (1H, ddd, J=8.8, 4.9, 3.7 Hz, H2), 3.90-3.86 (2H, m, H3, H6a), 3.85 (1H, d, J=8.3 Hz, H4), 3.79 (1H, ddd, J=8.3, 6.4, 1.0 Hz, H5), 3.69 (1H, dd, J=11.6, 6.4 Hz, H6b), 3.46 (1H, dd, J=14.6, 3.7 Hz, H1a), 3.06 (1H, dd, J=14.6, 8.8 Hz, H1b); ¹³C NMR (D₂O, 150 MHz) δ 161.1, 156.7, 133.4, 131.3, 128.5, 126.6, 125.9 (q, J_{F-C}=33.0 Hz), 125.2, 116.8, 72.3, 70.9, 69.2, 68.9, 63.1, 37.7; ¹⁹F NMR (D₂O, 470 MHz) δ −62.7; HRMS (ESI) calcd for C₁₅H₁₇F₃N₂O₆: 378.104; found: m/z 359.144 [M−H₂O+H]⁺.

Sugar-5ClBIMs:

### (1R,2S,3R)-1-(5-chloro-1H-benzo[d]imidazole-2-yl)butane-1,2,3,4-tetraol

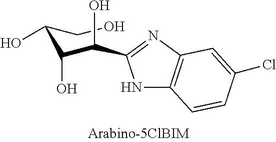

D-/L-arabinose (10 mg) and 4-chlorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give AraClBIM. The supporting data is given below.

C₁₁H₁₃ClN₂O₄; brown powder; mp=220-222° C.; [α]²⁵_{D}−30.3 (c 0.01, DMSO); ¹H NMR (DMSO-d6, 600 MHz) δ 7.52 (1H, d, J=2.0 Hz, ArH), 7.48 (1H, d, J=8.5 Hz, ArH), 7.15 (1H, dd, J=8.5, 2.0 Hz, ArH), 5.10 (1H, d, J=3.8 Hz, H1), 3.75 (1H, ddd, J=5.8, 3.1, 2.8 Hz, H3), 3.64 (1H, dd, J=10.9, 3.1 Hz, H4a), 3.61 (1H, dd, J=3.8, 2.8 Hz, H2), 3.44 (1H, dd, J=10.9, 5.8 Hz, H4b); ¹³C NMR (DMSO-d6, 150 MHz) δ 159.2, 139.6, 136.5, 125.6, 121.5, 115.8, 114.5, 73.9, 71.0, 67.6, 63.5; MS (MALDI-TOF) calcd for C₁₁H₁₃ClN₂O₄Na: 293.0564; found: m/z 292.926 [M+Na]⁺.

### (1S,2R,3S,4R)-1-(5-chloro-1H-benzo[d]imidazole-2-yl)pentane-1,2,3,4-tetraol

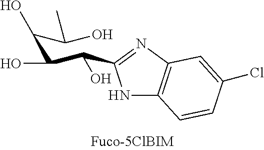

D-/L-fucose (10 mg) and 4-chlorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give FucClBIM. The supporting data is given below.

C₁₂H₁₅ClN₂O₄; gray powder; mp=258-260° C.; [α]²⁵_{D}−35.0 (c 0.01, DMSO); ¹H NMR (DMSO-d6, 600 MHz) δ 7.51 (1H, d, J=1.9 Hz, ArH), 7.48 (1H, d, J=8.4 Hz, ArH), 7.14 (1H, dd, J=8.4, 1.9 Hz, ArH), 5.12 (1H, d, J=5.7 Hz, H1), 4.19 (1H, dd, J=5.7, 5.2 Hz, H2), 3.92 (1H, dq, J=7.7, 6.4 Hz, H4), 3.88 (1H, dd, J=7.7, 5.2 Hz, H3), 1.13 (3H, d, J=6.4 Hz, H5); ¹³C NMR (DMSO-d6, 150 MHz) δ 159.5, 138.5 (2×), 125.6, 121.5, 115.4, 115.0, 73.3, 72.6, 67.8, 65.3, 20.1; MS (MALDI-TOF) calcd for C₁₂H₁₆ClN₂O₄: 287.072; found: m/z 286.952 [M+H]⁺.

### (1S,2R,3S,4R)-1-(5-chloro-1H-benzo[d]imidazole-2-yl)pentane-1,2,3,4,5-pentaol

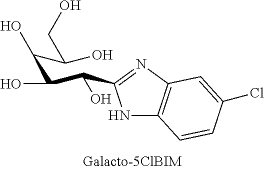

D-/L-galactose (10 mg) and 4-chlorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GalClBIM. The supporting data is given below.

C₁₂H₁₅ClN₂O₅; gray powder; mp=220-222° C.; [α]²⁵_{D}+39.3 (c 0.01, DMSO); ¹H NMR (DMSO-d6, 600 MHz) δ 7.52 (1H, d, J=1.9 Hz, ArH), 7.49 (1H, d, J=8.5 Hz, ArH), 7.15 (1H, dd, J=8.5, 1.9 Hz, ArH), 5.13 (1H, d, J=4.6 Hz, H1), 3.91 (1H, dd, J=7.8, 4.6 Hz, H2), 3.75 (1H, ddd, J=9.3, 6.3, 6.0 Hz, H4), 3.62 (1H, dd, J=9.3, 7.8 Hz, H3), 3.47 (1H, dd, J=10.3, 6.1 Hz, H5a), 3.43 (1H, dd, J=10.3, 6.3 Hz, H5b); ¹³C NMR (DMSO-d6, 150 MHz) δ 159.6, 139.7, 136.8, 125.8, 121.6, 115.9, 114.7, 72.9, 70.0, 69.3, 67.7, 63.3; MS (MALDI-TOF) calcd for C₁₂H₁₆ClN₂O₅: 303.067; found: m/z 302.949 [M+H]⁺.

### (1S,2R,3S)-1-(5-chloro-1H-benzo[d]imidazole-2-yl)-1,2,3,4-tetrtahydroxy pentanoic acid

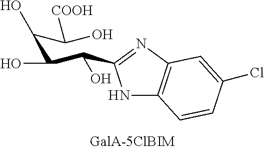

D-galacturonic acid (10 mg) and 4-chlorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GalAClBIM. The supporting data is given below.

C₁₂H₁₃ClN₂O₆; black powder; mp=138-140° C.; [α]²⁵_{D}+12.4 (c 0.01, DMSO); ¹H NMR (DMSO-d6, 600 MHz) δ 7.56 (1H, d, J=1.4 Hz, ArH), 7.52 (1H, d, J=8.5 Hz, ArH), 7.20 (1H, dd, J=8.5, 1.4 Hz, ArH), 5.14 (1H, d, J=0.6 Hz, H1), 4.30 (1H, d, J=0.7 Hz, H4), 3.96 (1H, dd, J=9.8, 0.7 Hz, H3), 3.93 (1H, dd, J=9.8, 0.6 Hz, H2); ¹³C NMR (DMSO-d6, 150 MHz) δ 175.5, 159.2, 138.7, 136.1, 126.3, 122.1, 115.8, 114.5, 72.5, 71.5, 70.1, 67.2; MS (MALDI-TOF) calcd for C₁₂H₁₄ClN₂O₆: 317.046; found: m/z 316.998 [M+H]⁺.

### (1S,2R,3R,4R)-1-(5-chloro-1H-benzo[d]imidazole-2-yl)pentane-1,2,3,4,5-pentaol

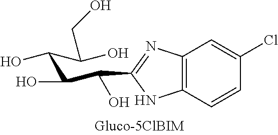

D-/L-glucose (10 mg) and 4-chlorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GlcClBIM. The supporting data is given below.

C₁₂H₁₅ClN₂O₅; black powder; mp=182-184° C.; [α]²⁵_{D}+30.3 (c 0.001, DMSO); ¹H NMR (DMSO-d6, 600 MHz) δ 7.57 (1H, d, J=1.8 Hz, ArH), 7.53 (1H, d, J=8.2 Hz, ArH), 7.21 (1H, dd, J=8.2, 1.8 Hz, ArH), 4.94 (1H, d, J=5.7 Hz, H1), 4.05 (1H, dd, J=5.7, 1.0 Hz, H2), 3.55 (1H, dd, J=10.8, 3.2 Hz, H5a), 3.50 (1H, ddd, J=8.5, 5.6, 3.2 Hz, H4), 3.44 (1H, dd, J=8.5, 1.0 Hz, H3), 3.34 (1H, dd, J=10.8, 5.6 Hz, H5b); ¹³C NMR (DMSO-d6, 150 MHz) δ 157.6, 138.3, 135.7, 126.5, 122.4, 115.9, 114.6, 71.9, 71.4, 70.8, 69.5, 63.5; MS (MALDI-TOF) calcd for C₁₂H₁₆ClN₂O₅: 303.067; found: m/z 302.948 [M+H]⁺.

### (1S,2R,3R)-1-(5-chloro-1H-benzo[d]imidazole-2-yl)-1,2,3,4-tetrtahydroxy pentanoic acid

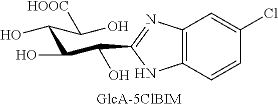

D-glucuronic acid (10 mg) and 4-chlorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GlcAClBIM. The supporting data is given below.

C₁₂H₁₃ClN₂O₆; yellow-brown powder; mp=168-170° C.; [α]²⁵_{D}+4.9 (c 0.01, DMSO); ¹H NMR (DMSO-d6, 600 MHz) δ 7.53 (1H, d, J=1.8 Hz, ArH), 7.49 (1H, d, J=8.5 Hz, ArH), 7.16 (1H, dd, J=8.5, 1.8 Hz, ArH), 4.90 (1H, d, J=6.1 Hz, H1), 3.96 (1H, dd, J=6.1, 2.1 Hz, H2), 3.90 (1H, d, 7.7 Hz, H4), 3.56 (1H, dd, J=7.7, 2.1 Hz, H3); ¹³C NMR (DMSO-d6, 150 MHz) δ 174.9, 157.5, 139.8, 137.1, 125.8, 121.7, 116.0, 114.8, 72.0, 71.9, 71.3, 69.5; MS (MALDI-TOF) calcd for C₁₂H₁₄ClN₂O₆: 317.046; found: m/z 317.021 [M+H]⁺.

### N-((1S,2R,3R,4R)-1-(5-chloro-1H-benzo[d]imidazol-2-yl)-2,3,4,5-tetrahydroxypentyl)-acetamide

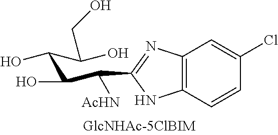

D-N-acetylglucosamine (10 mg) and 4-chlorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GlcNAcClBIM. The supporting data is given below.

C₁₄H₁₈ClN₃O₅; black powder; mp=˜74° C.; [α]²⁵_{D}−80.3 (c 0.001, DMSO); ¹H NMR (DMSO-d6, 600 MHz) δ 7.58 (1H, d, J=1.6 Hz, ArH), 7.52 (1H, d, J=8.5 Hz, ArH), 7.20 (1H, dd, J=8.5, 1.6 Hz, ArH), 5.20 (1H, t, J=2.0 Hz, H1), 4.23 (1H, dd, J=7.0, 2.0 Hz, H2), 3.56 (1H, J=11.0, 3.3 Hz, H5a), 3.48 (1H, ddd, J=8.5, 5.5, 3.3 Hz, H4), 3.38 (1H, J=11.0, 5.5 Hz, H5b), 3.26 (1H, dd, J=8.5, 7.0 Hz, H3), 1.90 (3H, s, Me); ¹³C NMR (DMSO-d6, 150 MHz) δ 169.9, 155.8, 138.9, 136.1, 126.5, 122.3, 115.9, 114.7, 71.4, 70.9, 70.6, 63.4, 51.5, 22.7; MS (MALDI-TOF) calcd for C₁₄H₁₉ClN₃O₅: 344.094; found: m/z 344.070 [M+H]⁺.

### (1R,2R,3R,4R)-1-(5-chloro-1H-benzo[d]imidazole-2-yl)pentane-1,2,3,4,5-pentaol

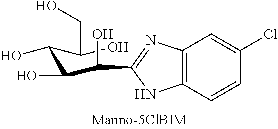

D-/L-mannose (10 mg) and 4-chlorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give ManClBIM. The supporting data is given below.

C₁₂H₁₅ClN₂O₅; gray powder; mp=196-198° C.; [α]²⁵_{D}−9.1 (c 0.01, DMSO); ¹H NMR (DMSO-d6, 600 MHz) δ 7.54 (1H, s, ArH), 7.50 (1H, d, J=8.4 Hz, ArH), 7.17 (1H, d, J=8.4 Hz, ArH), 4.76 (1H, d, J=7.8 Hz, H1), 4.04 (1H, d, J=7.8 Hz, H2), 3.66 (1H, dd, J=8.0 Hz, H3), 3.65 (1H, dd, J=11.0, 3.0 Hz, H5a), 3.51 (1H, ddd, J=8.0, 6.0, 3.0 Hz, H4), 3.42 (1H, dd, J=11.0, 6.0 Hz, H5b); ¹³C NMR (DMSO-d6, 150 MHz) δ 158.8, 139.5, 136.8, 125.9, 121.8, 116.0, 114.8, 71.6, 71.2, 69.8, 68.2, 63.9; MS (MALDI-TOF) calcd for C₁₂H₁₆ClN₂O₅: 303.067; found: m/z 302.943 [M+H]⁺.

### (1R,2R,3R,4R)-1-(5-chloro-1H-benzo[d]imidazole-2-yl)pentane-1,2,3,4-tetraol

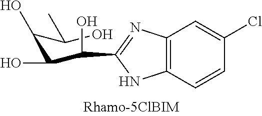

D-/L-rhamnose (10 mg) and 4-chlorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give RhaClBIM. The supporting data is given below.

C₁₂H₁₅ClN₂O₄; gray powder; mp=202-204° C.; [α]²⁵_{D}+90.0 (c 0.001, DMSO); ¹H NMR (DMSO-d6, 600 MHz) δ 7.55 (1H, d, J=1.9 Hz, ArH), 7.52 (1H, d, J=8.5 Hz, ArH), 7.14 (1H, dd, J=8.5, 1.9 Hz, ArH), 4.77 (1H, dd, J=8.0, 3.0 Hz, H1), 4.06 (1H, dd, J=8.0, 0.8 Hz, H2), 3.62 (1H, dq, J=8.2, 6.2 Hz, H4), 3.41 (1H, dd, J=8.2, 0.8 Hz, H3), 1.14 (3H, d, J=6.2 Hz, H5); ¹³C NMR (DMSO-d6, 150 MHz) δ 158.7, 139.0, 136.4, 126.1, 122.1, 116.0, 114.7, 73.7, 71.5, 68.3, 66.2, 20.9; MS (MALDI-TOF) calcd for C₁₂H₁₆ClN₂O₄: 286.072; found: m/z 287.037 [M+H]⁺.

### (1S,2S,3R)-1-(5-chloro-1H-benzo[d]imidazole-2-yl)butane-1,2,3,4-tetraol

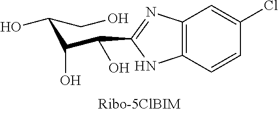

D-/L-ribose (10 mg) and 4-chlorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give RibClBIM. The supporting data is given below.

C₁₁H₁₃ClN₂O₄; black powder; [α]²⁵_{D}+60.3 (c 0.001, DMSO); ¹H NMR (DMSO-d6, 600 MHz) δ 7.71 (1H, d, J=1.8 Hz, ArH), 7.66 (1H, d, J=8.6 Hz, ArH), 7.41 (1H, dd, J=8.6, 1.8 Hz, ArH), 5.14 (1H, d, J=4.5 Hz, H1), 3.87 (1H, dd, J=7.0, 4.5 Hz, H2), 3.58 (1H, dd, J=10.9, 3.5 Hz, H4a), 3.51 (1H, ddd, J=7.0, 6.0, 3.5 Hz, H3), 3.44 (1H, dd, J=10.9, 6.0 Hz, H4b); ¹³C NMR (DMSO-d6, 150 MHz) δ 156.8, 134.1, 132.1, 128.6, 124.6, 115.8, 114.1, 74.0, 71.8, 68.0, 62.9; MS (MALDI-TOF) calcd for C₁₁H₁₄ClN₂O₄: 273.056; found: m/z 272.990 [M+H]⁺.

### (1S,2R,3R)-1-(5-chloro-1H-benzo[d]imidazole-2-yl)butane-1,2,3,4-tetraol

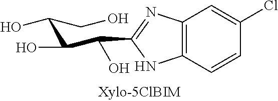

D-/L-xylose (10 mg) and 4-chlorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give XylClBIM. The supporting data is given below.

C₁₁H₁₃ClN₂O₄; black powder; [α]²⁵_{D}+20.0 (c 0.001, DMSO); ¹H NMR (DMSO-d6, 600 MHz) δ 7.64 (1H, d, J=1.6 Hz, ArH), 7.61 (1H, d, J=8.5 Hz, ArH), 7.33 (1H, dd, J=8.5, 1.6 Hz, ArH), 5.04 (1H, d, J=4.8 Hz, H1), 3.91 (1H, dd, J=4.8, 3.5 Hz, H2), 3.62 (1H, ddd, J=6.2, 5.8, 3.5 Hz, H3), 3.48 (1H, dd, J=10.7, 6.2 Hz, H4a), 3.38 (1H, dd, J=10.7, 5.8 Hz, H4b); ¹³C NMR (DMSO-d6, 150 MHz) δ 157.7, 136.1, 133.8, 127.6, 123.6, 115.8, 114.3, 72.5, 70.9, 68.2, 62.4; MS (MALDI-TOF) calcd for C₁₁H₁₃ClN₂O₄: 273.056; found: m/z 272.990 [M+H]⁺; calcd for C₁₁H₁₃ClN₂O₄Na: 286.056; found: m/z 286.026 [M+Na]⁺.

Compounds: Sugar-5,6Cl₂BIMs

### (1R,2S,3R)-1-(5,6-dichloro-1H-benzo[d]imidazole-2-yl)butane-1,2,3,4-tetraol

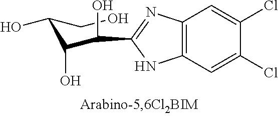

D-/L-arabinose (10 mg) and 4,5-dichlorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give AraCl2BIM. The supporting data is given below.

C₁₁H₁₂Cl₂N₂O₄; purple powder; mp=234-236° C.; [α]²⁵_{D}−27.3 (c 0.01, DMSO); ¹H NMR (DMSO-d6, 600 MHz) δ 7.71 (2H, s, ArH), 5.11 (1H, s, H1), 3.76 (1H, ddd, J=5.5, 3.2, 2.8 Hz, H3), 3.64 (1H, d, J=2.8 Hz, H2), 3.63 (1H, dd, J=10.6, 3.2 Hz, H4a), 3.45 (1H, dd, J=10.6, 5.5 Hz, H4b); ¹³C NMR (DMSO-d6, 150 MHz) δ 160.7, 139.9 (2×), 123.5 (2×), 116.0 (2×), 73.8, 70.9, 67.6, 63.5; MS (MALDI-TOF) calcd for C₁₁H₁₃Cl₂N₂O₄: 307.017; found: m/z 306.979 [M+H]⁺; calcd for C₁1H12Cl2N2O4Na: 329.017; found: m/z 328.971 [M+Na]+.

### (1S,2R,3S,4R)-1-(5,6-dichloro-1H-benzo[d]imidazole-2-yl)pentane-1,2,3,4-tetraol

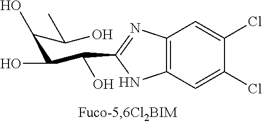

D-/L-fucose (10 mg) and 4,5-dichlorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give FucCl2BIM. The supporting data is given below.

C₁₂H₁₄Cl₂N₂O₄; yellow-white powder; mp=260° C., decomposed; [α]²⁵_{D}+36.03 (c 0.01, DMSO); ¹H NMR (DMSO-d6, 600 MHz) δ 7.85 (2H, s, ArH), 5.24 (1H, s, H1), 4.25 (1H, d, J=7.0 Hz, H2), 3.92 (1H, dq, J=6.6, 6.2 Hz, H4), 3.69 (1H, dd, J=7.0, 6.6 Hz, H3), 1.13 (3H, d, J=6.2 Hz, H5); ¹³C NMR (DMSO-d6, 150 MHz) δ 160.5, 135.8 (2×), 119.4 (2×), 113.1 (2×), 73.6, 72.4, 69.7, 65.0, 20.1; MS (MALDI-TOF) calcd for C₁₂H₁₅Cl₂N₂O₄: 321.033; found: m/z 321.000 [M+H]⁺; calcd for C₁₂H₁₄Cl₂N₂O₄Na: 343.033; found: m/z 342.975 [M+Na]⁺.

### (1S,2R,3S,4R)-1-(5,6-dichloro-1H-benzo[d]imidazol-2-yl)pentane-1,2,3,4,5-pentaol

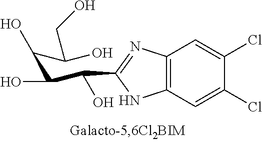

D-/L-galactose (10 mg) and 4,5-dichlorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GalCl2BIM. The supporting data is given below.

C₁₂H₁₄Cl₂N₂O₅; gray powder; mp=260° C., decomposed; [α]²⁵_{D}+7.4 (c 0.01, DMSO); ¹H NMR (DMSO-d6, 600 MHz) δ 7.74 (2H, s, ArH), 5.17 (1H, d, J=1.3 Hz, H1), 3.92 (1H, dd, J=9.6, 1.3 Hz, H2), 3.75 (1H, ddd, J=6.7, 6.5, 0.9 Hz, H4), 3.63 (1H, dd, J=9.5, 0.9 Hz, H3), 3.46 (1H, dd, J=10.6, 6.5 Hz, H5a), 3.43 (1H, dd, J=10.6, 6.7 Hz, H5b); ¹³C NMR (DMSO-d6, 150 MHz) δ 160.9, 137.6 (2×), 123.9 (2×), 115.9 (2×), 72.8, 69.8, 69.1, 67.6, 63.1; MS (MALDI-TOF) calcd for C₁₂H₁₅Cl₂N₂O₅: 337.028; found: m/z 336.994 [M+H]⁺; calcd for C₁₂H₁₄Cl₂N₂O₅Na: 359.028; found: m/z 358.985 [M+Na]⁺.

### (1S,2R,3S)-1-(5,6-dichloro-1H-benzo[d]imidazole-2-yl)-1,2,3,4-tetrtahydroxy pentanoic acid

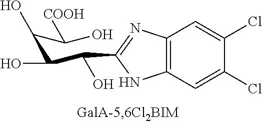

D-galacturonic acid (10 mg) and 4,5-dichlorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GalACl2BIM. The supporting data is given below.

C₁₂H₁₂Cl₂N₂O₆; gray powder; mp=136-138° C.; [α]²⁵_{D}+15.3 (c 0.01, DMSO); ¹H NMR (DMSO-d6, 600 MHz) δ 7.78 (2H, s, ArH), 5.17 (1H, d, J=0.9 Hz, H1), 4.30 (1H, d, J=1.1 Hz, H4), 3.97 (1H, dd, J=9.7, 1.1 Hz, H3), 3.93 (1H, dd, J=9.7, 0.9 Hz, H2); ¹³C NMR (DMSO-d6, 150 MHz) δ 175.4, 160.5, 136.7 (2×), 124.5 (2×), 115.9 (2×), 72.4, 71.4, 69.9, 67.2; MS (MALDI-TOF) calcd for C₁₂H₁₃Cl₂N₂O₆: 350.007; found: m/z 350.008 [M+H]⁺; calcd for C₁₂H₁₂Cl₂N₂O₆Na: 373.007; found: m/z 373.000 [M+Na]⁺.

### (1S,2R,3R,4R)-1-(5,6-dichloro-1H-benzo[d]imidazole-2-yl)pentane-1,2,3,4,5-pentaol

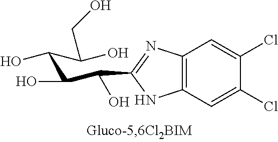

D-/L-glucose (10 mg) and 4,5-dichlorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GlcCl2BIM. The supporting data is given below.

C₁₂H₁₄Cl₂N₂O₅; brown powder; mp=160-162° C.; [α]²⁵_{D}+70.3 (c 0.01, DMSO); ¹H NMR (DMSO-d6, 600 MHz) δ 7.76 (2H, s, ArH), 4.98 (1H, d, J=5.5 Hz, H1), 4.07 (1H, dd, J=5.5, 1.0 Hz, H2), 3.70 (1H, dd, J=11.0, 3.2 Hz, H5a), 3.63 (1H, ddd, J=8.5, 5.6, 3.2 Hz, H4), 3.44 (1H, dd, J=8.5, 1.0 Hz, H3), 3.35 (1H, dd, J=11.0, 5.6 Hz, H5b); ¹³C NMR (DMSO-d6, 150 MHz) δ 161.3, 139.9 (2×), 123.3 (2×), 118.6 (2×), 74.3, 72.6, 71.1, 71.0, 63.4; MS (MALDI-TOF) calcd for C₁₂H₁₅Cl₂N₂O₅: 337.028; found: m/z 336.995 [M+H]⁺; calcd for C₁₂H₁₄Cl₂N₂O₅Na: 359.028; found: m/z 358.984 [M+Na]⁺.

### (1S,2R,3R)-1-(5,6-dichloro-1H-benzo[d]imidazole-2-yl)-1,2,3,4-tetrtahydroxy pentanoic acid

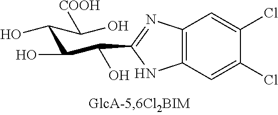

D-glucuronic acid (10 mg) and 4,5-dichlorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GlcACl2BIM. The supporting data is given below.

C₁₂H₁₂Cl₂N₂O₆; yellow-brownish powder; mp=134-136° C.; [α]²⁵_{D}+6.1 (c 0.01, DMSO); ¹H NMR (DMSO-d6, 600 MHz) δ 7.73 (2H, s, ArH), 4.92 (1H, d, J=5.8 Hz, H1), 3.97 (1H, dd, J=5.8, 2.5 Hz, H2), 3.95 (1H, d, J=7.7 Hz, H4), 3.60 (1H, dd, J=7.7, 2.5 Hz, H3); ¹³C NMR (DMSO-d6, 150 MHz) δ 174.7, 158.9, 138.3 (2×), 123.7 (2×), 116.2 (2×), 71.9, 71.8, 71.4, 69.4; MS (MALDI-TOF) calcd for C₁₂H₁₃Cl₂N₂O₆: 350.007; found: m/z 350.048 [M+H]⁺.

### N-((1S,2R,3R,4R)-1-(5,6-dichloro-1H-benzo[d]imidazol-2-yl)-2,3,4,5-tetrahydroxypentyl)-acetamide

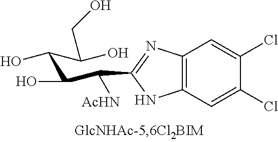

D-N-acetylglucosamine (10 mg) and 4,5-dichlorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GlcNAcCl2BIM. The supporting data is given below.

C₁₄H₁₇Cl₂N₃O₅; black syrup; [α]²⁵_{D}+7.3 (c 0.01, DMSO); ¹H NMR (DMSO-d6, 600 MHz) δ 7.95 (1H, s, ArH), 5.24 (1H, t, J=2.7 Hz, H1), 4.23 (1H, dd, J=5.0, 2.7 Hz, H2), 3.56 (1H, J=11.0, 3.3 Hz, H5a), 3.48 (1H, ddd, J=8.5, 5.5, 3.3 Hz, H4), 3.38 (1H, J=11.0, 5.5 Hz, H5b), 3.26 (1H, dd, J=8.5, 5.0 Hz, H3), 1.99 (3H, s, Me); ¹³C NMR (DMSO-d6, 150 MHz) δ 169.8, 156.8, 135.0 (2×), 126.2 (2×), 115.6 (2×), 71.1, 70.9, 70.3, 63.2, 51.4, 22.6; MS (MALDI-TOF) calcd for C₁₄H₁₈Cl₂N₃O₅: 378.055; found: m/z 378.048 [M+H]⁺; calcd for C₁₄H₁₇Cl₂N₃O₅Na: 400.055; found: m/z 400.048 [M+Na]⁺.

### (1R,2R,3R,4R)-1-(5,6-dichloro-1H-benzo[d]imidazole-2-yl)pentane-1,2,3,4,5-pentaol

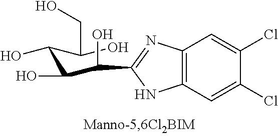

D-/L-mannose (10 mg) and 4,5-dichlorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give ManCl2BIM. The supporting data is given below.

C₁₂H₁₄Cl₂N₂O₅; purple powder; mp=194-196° C.; [α]²⁵_{D}−11.3 (c 0.01, DMSO); ¹H NMR (DMSO-d6, 600 MHz) δ 7.74 (2H, s, ArH), 4.77 (1H, d, J=8.4 Hz, H1), 4.05 (1H, d, J=8.4 Hz, H2), 3.67 (1H, d, J=9.0 Hz, H3), 3.65 (1H, dd, J=10.9, 3.3 Hz, H5a), 3.52 (1H, ddd, J=9.0, 6.0, 3.3 Hz, H4), 3.43 (1H, dd, J=10.9, 6.0 Hz, H5b); ¹³C NMR (DMSO-d6, 150 MHz) δ 160.1, 138.2 (2×), 123.8 (2×), 116.1 (2×), 71.5, 71.1, 69.7, 68.0, 63.8; MS (MALDI-TOF) calcd for C₁₂H₁₅Cl₂N₂O₅: 337.028; found: m/z 336.983 [M+H]⁺; calcd for C₁₂H₁₄Cl₂N₂O₅Na: 359.028; found: m/z 358.969 [M+Na]⁺.

### (1R,2R,3R,4R)-1-(5,6-dichloro-1H-benzo[d]imidazole-2-yl)pentane-1,2,3,4-tetraol

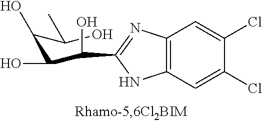

D-/L-rhamnose (10 mg) and 4,5-dichlorophenyldiamine (10 mg) with catalytic amount of iodine (1 mg) in acetic acid (1 mL) was mixed at room temperature for 12 hour. The resulting solution was precipitated by ethyl acetate (30 mL) and centrifuged for three times to remove the excess reagent. The pellet was lyophilized to give RhaCl2BIM. The supporting data is given below.

C₁₂H₁₄Cl₂N₂O₄; orange powder; mp=80-82° C.; [α]²⁵_{D}+45.0 (c 0.01, DMSO); ¹H NMR (DMSO-d6, 600 MHz) δ 8.04 (2H, s, ArH), 4.96 (1H, d, J=8.8 Hz, H1), 4.02 (1H, d, J=8.8 Hz, H2), 3.64 (1H, dq, J=8.6, 6.2 Hz, H4), 3.42 (1H, d, J=8.6 Hz, H3), 1.18 (3H, d, J=6.2 Hz, Me); ¹³C NMR (DMSO-d6, 150 MHz) δ 159.3, 131.8 (2×), 127.7 (2×), 115.7 (2×), 73.3, 71.2, 66.8, 65.8, 21.0; MS (MALDI-TOF) calcd for C₁₂H₁₅Cl₂N₂O₄: 321.033; found: m/z 321.042 [M+H]⁺; calcd for C₁₂H₁₄Cl₂N₂O₄Na: 343.033; found: m/z 343.020 [M+Na]⁺.

### (1S,2S,3R)-1-(5,6-dichloro-1H-benzo[d]imidazole-2-yl)butane-1,2,3,4-tetraol

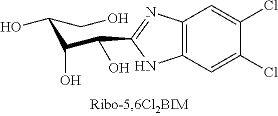

D-/L-ribose (10 mg) and 4,5-dichlorophenyldiamine (10 mg) with catalytic amount of iodine (I mg) in acetic acid (1 mL) was reacted at room temperature for 12 hour. The resulting solution was precipitated by ethyl acetate (30 mL) and centrifuged for three times to remove the excess reagent. The pellet was lyophilized to give RibCl2BIM. The supporting data is given below.

C₁₁H₁₂Cl₂N₂O₄; black powder; mp=220-222° C.; [α]²⁵_{D}+23.3 (c 0.01, DMSO); ¹H NMR (DMSO-d6, 600 MHz) δ 7.74 (1H, s, ArH), 5.01 (1H, d, J=4.4 Hz, H1), 3.82 (1H, dd, J=6.7, 4.4 Hz, H2), 3.55 (1H, dd, J=11.0, 3.5 Hz, H4a), 3.52 (1H, ddd, J=6.7, 5.8, 3.5 Hz, H3), 3.43 (1H, dd, J=11.0, 5.8 Hz, H4b); ¹³C NMR (DMSO-d6, 150 MHz) δ 158.8, 137.7 (2×), 123.7 (2×), 116.2 (2×), 74.7, 71.9, 69.1, 63.2; MS (MALDI-TOF) calcd for C₁₁H₁₃Cl₂N₂O₄: 307.017; found: m/z 306.961 [M+H]⁺; calcd for C₁₁H₁₂Cl₂N₂O₄Na: 329.017; found: m/z 328.951 [M+Na]⁺.

### (1S,2R,3R)-1-(5,6-dichloro-1H-benzo[d]imidazole-2-yl)butane-1,2,3,4-tetraol

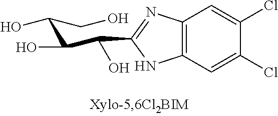

D-/L-xylose (10 mg) and 4,5-dichlorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give XylCl2BIM. The supporting data is given below.

C₁₁H₁₂Cl₂N₂O₄; black syrup; [α]²⁵_{D}−11.0 (c 0.01, DMSO); ¹H NMR (DMSO-d6, 600 MHz) δ 7.81 (2H, s, ArH), 5.00 (1H, d, J=4.6 Hz, H1), 3.92 (1H, dd, J=4.6, 3.1 Hz, H2), 3.64 (1H, ddd, J=6.2, 5.9, 3.1 Hz, H3), 3.47 (1H, dd, J=10.7, 6.2 Hz, H4a), 3.39 (1H, dd, J=10.7, 5.9 Hz, H4b); ¹³C NMR (DMSO-d6, 150 MHz) δ 159.2, 135.4 (2×), 123.7 (2×), 116.2 (2×), 72.6, 71.1, 68.4, 62.3; MS (MALDI-TOF) calcd for C₁₁H₁₂Cl₂N₂O₄Na: 329.017; found: m/z 328.919 [M+Na]⁺.

Sugar-5BrBIMs

### (1R,2S,3R)-1-(5-bromo-1H-benzo[d]imidazole-2-yl)butane-1,2,3,4-tetraol

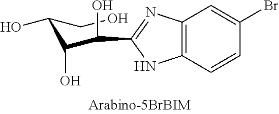

D-/L-arabinose (10 mg) and 4-bromophenyldiamine (10 mg) with catalytic amount of iodine (1 mg) in acetic acid (1 mL) was mixed at room temperature for 12 hour. The resulting solution was precipitated by ethyl acetate (30 mL) and centrifuged for three times to remove the excess reagent. The pellet was lyophilized to give AraBrBIM. The supporting data is given below.

C₁₁H₁₃BrN₂O₄; purple powder; mp=228-230° C.; [α]²⁵_{D}−35.3 (c 0.01, DMSO); ¹H NMR (DMSO-d6, 600 MHz) δ 7.66 (1H, d, J=1.5 Hz, ArH), 7.44 (1H, d, J=8.5 Hz, ArH), 7.25 (1H, dd, J=8.5, 1.5 Hz, ArH), 5.10 (1H, d, J=3.5 Hz, H1), 3.76 (1H, ddd, J=5.8, 3.2, 2.8 Hz, H3), 3.63 (1H, dd, J=10.9, 3.2 Hz, H4a), 3.62 (1H, dd, J=3.5, 2.8 Hz, H2), 3.45 (1H, dd, J=10.9, 5.8 Hz, H4b); ¹³C NMR (DMSO-d6, 150 MHz) δ 159.0, 137.1, 134.9, 123.9, 117.5, 116.9, 113.3, 73.8, 71.0, 67.5, 63.5; MS (MALDI-TOF) calcd for C₁₁H₁₄BrN₂O₄: 317.006; found: m/z 316.913 [M+H]⁺; calcd for C₁₁H₁₃BrN₂O₄Na: 339.006; found: m/z 338.908 [M+Na]⁺.

### (1S,2R,3S,4R)-1-(5-bromo-1H-benzo[d]imidazole-2-yl)pentane-1,2,3,4-tetraol

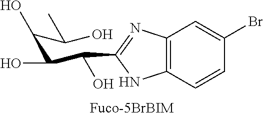

D-/L-fucose (10 mg) and 4-bromophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give FucBrBIM. The supporting data is given below.

C₁₂H₁₅BrN₂O₄; yellow-white powder; mp=258-260° C.; [α]²⁵_{D}−34.0 (c 0.01, DMSO); ¹H NMR (DMSO-d6, 600 MHz) δ 7.66 (1H, d, J=1.9 Hz, ArH), 7.44 (1H, d, J=8.4 Hz, ArH), 7.27 (1H, dd, J=8.4, 1.9 Hz, ArH), 5.13 (1H, d, J=5.7 Hz, H1), 4.19 (1H, dd, J=5.7, 5.2 Hz, H2), 3.92 (1H, dq, J=7.7, 6.4 Hz, H4), 3.90 (1H, dd, J=7.7, 5.2 Hz, H3), 1.13 (3H, d, J=6.4 Hz, H5); ¹³C NMR (DMSO-d6, 150 MHz) δ 159.3, 138.5 (2×), 123.9, 117.0 (2×), 113.3, 73.2, 72.5, 67.6, 65.2, 20.1; MS (MALDI-TOF) calcd for C₁₂H₁₆BrN₂O₄: 331.022; found: m/z 330.948 [M+H]⁺; calcd for C₁₂H₁₅BrN₂O₄Na: 353.022; found: m/z 352.940 [M+Na]⁺.

### (1S,2R,3S,4R)-1-(5-bromo-1H-benzo[d]imidazole-2-yl)pentane-1,2,3,4,5-pentaol

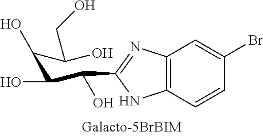

D-/L-galactose (10 mg) and 4-bromophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to giveGalBrBIM. The supporting data is given below.

C₁₂H₁₅BrN₂O₅; gray powder; mp=218-220° C.; [α]²⁵_{D}+27.3 (c 0.01, DMSO); ¹H NMR (DMSO-d6, 600 MHz) δ 7.69 (1H, d, J=1.7 Hz, ArH), 7.47 (1H, d, J=8.5 Hz, ArH), 7.30 (1H, dd, J=8.5, 1.7 Hz, ArH), 5.17 (1H, s, H1), 3.93 (1H, d, J=9.4 Hz, H2), 3.76 (1H, ddd, J=6.8, 6.6, 6.3 Hz, H4), 3.64 (1H, dd, J=9.4, 6.8 Hz, H3), 3.47 (1H, dd, J=10.5, 6.3 Hz, H5a), 3.43 (1H, dd, J=10.5, 6.6 Hz, H5b); ¹³C NMR (DMSO-d6, 150 MHz) δ 159.4, 139.4, 136.6, 124.8, 117.6, 116.4, 114.1, 73.0, 70.0, 69.3, 68.0, 63.3; MS (MALDI-TOF) calcd for C₁₂H₁₆BrN₂O₅: 347.016; found: m/z 346.949 [M+H]⁺; calcd for C₁₂H₁₅BrN₂O₅Na: 369.016; found: m/z 368.949 [M+Na]⁺.

### (1S,2R,3S)-1-(5-bromo-1H-benzo[d]imidazole-2-yl)-1,2,3,4-tetrtahydroxy pentanoic acid

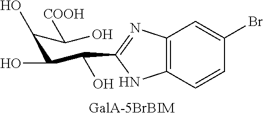

D-galacturonic acid (10 mg) and 4-bromophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GalABrBIM. The supporting data is given below.

C₁₂H₁₃BrN₂O₆; brownish powder; mp=132-134° C.; [α]²⁵_{D}+11.3 (c 0.01, DMSO); ¹H NMR (DMSO-d6, 600 MHz) δ 7.73 (1H, d, J=1.8 Hz, ArH), 7.52 (1H, d, J=8.6 Hz, ArH), 7.37 (1H, dd, J=8.6, 1.8 Hz, ArH), 5.18 (1H, d, J=0.6 Hz, H1), 4.30 (1H, d, J=0.7 Hz, H4), 3.97 (1H, dd, J=10.3, 0.7 Hz, H3), 3.93 (1H, dd, J=10.3, 0.6 Hz, H2); ¹³C NMR (DMSO-d6, 150 MHz) δ 174.7, 157.3, 140.3, 137.3, 124.2, 117.6, 116.4, 113.5, 71.8, 71.7, 71.4, 69.4; MS (MALDI-TOF) calcd for C₁₂H₁₄BrN₂O₆: 360.996; found: m/z 361.029 [M+H]⁺.

### (1S,2R,3R,4R)-1-(5-bromo-1H-benzo[d]imidazole-2-yl)pentane-1,2,3,4,5-pentaol

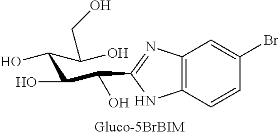

D-/L-glucose (10 mg) and 4-bromophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GlcBrBIM. The supporting data is given below.

C₁₂H₁₅BrN₂O₅; gray powder; mp=156-158° C.; [α]²⁵_{D}+18.3 (c 0.01, DMSO); ¹H NMR (DMSO-d6, 600 MHz) δ 7.76 (1H, d, J=1.8 Hz, ArH), 7.55 (1H, d, J=8.5 Hz, ArH), 7.44 (1H, dd, J=8.5, 1.8 Hz, ArH), 5.02 (1H, d, J=5.7 Hz, H1), 4.11 (1H, dd, J=5.7, 1.5 Hz, H2), 3.55 (1H, dd, J=10.9, 3.2 Hz, H5a), 3.51 (1H, ddd, J=8.5, 5.6, 3.2 Hz, H4), 3.44 (1H, dd, J=8.5, 1.5 Hz, H3), 3.35 (1H, dd, J=10.9, 5.6 Hz, H5b); ¹³C NMR (DMSO-d6, 150 MHz) δ 157.3, 136.6, 134.0, 126.0, 117.1, 116.2, 115.2, 71.6, 71.2, 70.1, 68.8, 63.3; MS (MALDI-TOF) calcd for C₁₂H₁₆BrN₂O₅: 347.016; found: m/z 346.952 [M+H]⁺; calcd for C₁₂H₁₅BrN₂O₅Na: 369.016; found: m/z 368.949 [M+Na]⁺.

### (1S,2R,3R)-1-(5-bromo-1H-benzo[d]imidazole-2-yl)-1,2,3,4-tetrtahydroxy pentanoic acid

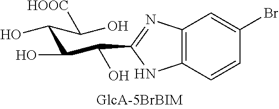

D-glucuronic acid (10 mg) and 4-bromophenyldiamine (10 mg) with catalytic amount of iodine (1 mg) in acetic acid (I mL) was reacted at room temperature for 12 hour. The resulting solution was precipitated by ethyl acetate (30 mL) and centrifuged for three times to remove the excess reagent. The pellet was lyophilized to give GlcABrBIM. The supporting data is given below.

C₁₂H₁₃BrN₂O₆; gray powder; mp=152-154° C.; [α]²⁵_{D}+15.3 (c 0.01, DMSO); ¹H NMR (DMSO-d6, 600 MHz) δ 7.67 (1H, d, J=1.2 Hz, ArH), 7.46 (1H, d, J=8.5 Hz, ArH), 7.28 (1H, dd, J=8.5, 1.6 Hz, ArH), 4.91 (1H, d, J=6.0 Hz, H1), 3.96 (1H, dd, J=6.0, 2.1 Hz, H2), 3.95 (1H, d, 7.8 Hz, H4), 3.58 (1H, dd, J=7.8, 2.1 Hz, H3); ¹³C NMR (DMSO-d6, 150 MHz) δ 174.7, 157.3, 140.6, 137.2, 124.2, 117.7, 116.5, 113.5, 71.8, 71.7, 71.4, 69.4; MS (MALDI-TOF) calcd for C₁₂H₁₄BrN₂O₆: 360.996; found: m/z 361.000 [M+H]⁺.

### N-((1S,2R,3R,4R)-1-(5-bromo-1H-benzo[d]imidazol-2-yl)-2,3,4,5-tetrahydroxypentyl)-acetamide

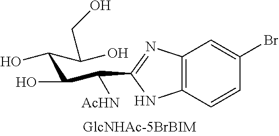

D-N-acetylglucosamine (10 mg) and 4-bromophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GlcNAcBrBIM. The supporting data is given below.

C₁₄H₁₈BrN₃O₅; black powder; moisturized; [α]²⁵_{D}+4.3 (c 0.01, DMSO); ¹H NMR (DMSO-d6, 600 MHz) δ 7.94 (1H, d, J=1.7 Hz, ArH), 7.78 (1H, d, J=8.7 Hz, ArH), 7.64 (1H, dd, J=8.7, 1.7 Hz, ArH), 5.24 (1H, t, J=2.5 Hz, H1), 4.23 (1H, dd, J=5.0, 2.5 Hz, H2), 3.56 (1H, J=11.0, 3.3 Hz, H5a), 3.48 (1H, ddd, J=8.5, 5.5, 3.3 Hz, H4), 3.38 (1H, J=11.0, 5.5 Hz, H5b), 3.26 (1H, dd, J=8.5, 5.0 Hz, H3), 2.00 (3H, s, Me); ¹³C NMR (DMSO-d6, 150 MHz) δ 169.7, 154.9, 132.9, 130.8, 128.1, 117.7, 117.2, 116.5, 71.0, 70.8, 70.0, 63.0, 51.3, 22.9; MS (MALDI-TOF) calcd for C₁₄H₁₉BrN₃O₅: 388.043; found: m/z 388.066 [M+H]⁺; calcd for C₁₄H₁₈BrN₃O₅Na: 410.043; found: m/z 410.064 [M+Na]⁺.

### (1R,2R,3R,4R)-1-(5-bromo-1H-benzo[d]imidazole-2-yl)pentane-1,2,3,4,5-pentaol

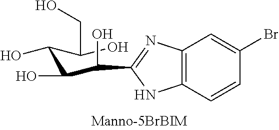

D-/L-mannose (10 mg) and 4-bromophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give ManBrBIM. The supporting data is given below.

C₁₂H₁₅BrN₂O₅; purple powder; mp=188-190° C.; [α]²⁵_{D}−20.3 (c 0.01, DMSO); ¹H NMR (DMSO-d6, 600 MHz) δ 7.69 (1H, d, J=1.8 Hz, ArH), 7.47 (1H, d, J=8.5 Hz, ArH), 7.31 (1H, dd, J=8.5, 1.9 Hz, ArH), 4.78 (1H, d, J=8.3 Hz, H1), 4.05 (1H, d, J=8.3 Hz, H2), 3.67 (1H, d, J=9.0 Hz, H3), 3.65 (1H, dd, J=11.0, 3.3 Hz, H5a), 3.52 (1H, ddd, J=9.0, 6.1, 3.3 Hz, H4), 3.43 (1H, dd, J=11.0, 6.1 Hz, H5b); ¹³C NMR (DMSO-d6, 150 MHz) δ 158.6, 139.8, 136.8, 124.6, 117.6, 116.4, 113.9, 71.6, 71.2, 69.8, 68.1, 63.9; MS (MALDI-TOF) calcd for C₁₂H₁₆BrN₂O₅: 347.016; found: m/z 346.947 [M+H]⁺; calcd for C₁₂H₁₅BrN₂O₅Na: 369.016; found: m/z 368.946 [M+Na]⁺.

### (1R,2R,3R,4R)-1-(5-bromo-1H-benzo[d]imidazole-2-yl)pentane-1,2,3,4-tetraol

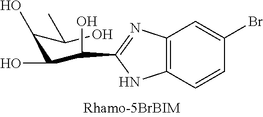

D-/L-rhamnose (10 mg) and 4-bromophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give RhaBrBIM. The supporting data is given below.

C₁₂H₁₅BrN₂O₄; orange powder; mp=166-168° C.; [α]²⁵_{D}+40.3 (c 0.01, DMSO); ¹H NMR (DMSO-d6, 600 MHz) δ 7.92 (1H, d, J=1.7 Hz, ArH), 7.68 (1H, d, J=8.6 Hz, ArH), 7.62 (1H, dd, J=8.6, 1.7 Hz, ArH), 4.94 (1H, d, J=8.8 Hz, H1), 4.02 (1H, dd, J=8.8, 0.6 Hz, H2), 3.63 (1H, dq, J=8.6, 6.2 Hz, H4), 3.42 (1H, dd, J=8.6, 0.6 Hz, H3), 1.17 (3H, d, J=6.2 Hz, H5); ¹³C NMR (DMSO-d6, 150 MHz) δ 157.8, 133.7, 131.5, 127.9, 117.0, 116.8, 116.0, 73.3, 71.2, 66.8, 65.8; MS (MALDI-TOF) calcd for C₁₂H₁₆BrN₂O₄: 331.022; found: m/z 331.052 [M+H]⁺; calcd for C₁₂H₁₅BrN₂O₄Na: 353.022; found: m/z 353.054 [M+Na]⁺.

### (1S,2S,3R)-1-(5-bromo-1H-benzo[d]imidazole-2-yl)butane-1,2,3,4-tetraol

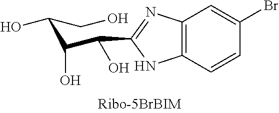

D-/L-ribose (10 mg) and 4-bromophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give RibBrBIM. The supporting data is given below.

C₁₁H₁₃BrN₂O₄; purple powder; mp=108-110° C.; [α]²⁵_{D}+40.0 (c 0.001, DMSO); ¹H NMR (DMSO-d6, 600 MHz) δ 7.83 (1H, d, J=0.8 Hz, ArH), 7.62 (1H, d, J=8.6 Hz, ArH), 7.51 (1H, dd, J=8.6, 0.8 Hz, ArH), 5.14 (1H, d, J=4.4 Hz, H1), 3.87 (1H, dd, J=6.7, 4.4 Hz, H2), 3.58 (1H, dd, J=11.0, 3.5 Hz, H4a), 3.51 (1H, ddd, J=6.7, 5.8, 3.5 Hz, H3), 3.44 (1H, dd, J=11.0, 5.8 Hz, H4b); ¹³C NMR (DMSO-d6, 150 MHz) δ 156.6, 135.2, 132.9, 126.8, 117.0, 116.1, 116.0, 74.0, 71.8, 68.1, 62.9; MS (MALDI-TOF) calcd for C₁₁H₁₄BrN₂O₄: 317.006; found: m/z 316.946 [M+H]⁺; calcd for C₁₁H₁₃BrN₂O₄Na: 339.006; found: m/z 338.930 [M+Na]⁺.

### (1S,2R,3R)-1-(5-bromo-1H-benzo[d]imidazole-2-yl)butane-1,2,3,4-tetraol

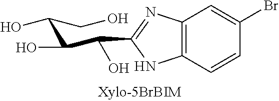

D-/L-xylose (10 mg) and 4-bromophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give XylBrBIM. The supporting data is given below.

C₁₁H₁₃BrN₂O₄; black powder; moisturized; [α]²⁵_{D}−4.3 (c 0.01, DMSO); ¹H NMR (DMSO-d6, 600 MHz) δ 7.85 (1H, d, J=1.6 Hz, ArH), 7.63 (1H, d, J=8.6 Hz, ArH), 7.58 (1H, dd, J=8.6, 1.6 Hz, ArH), 5.13 (1H, d, J=4.7 Hz, H1), 3.96 (1H, dd, J=4.7, 3.1 Hz, H2), 3.64 (1H, ddd, J=6.2, 5.9, 3.1 Hz, H3), 3.47 (1H, dd, J=10.7, 6.2 Hz, H4a), 3.39 (1H, dd, J=10.7, 5.9 Hz, H4b); ¹³C NMR (DMSO-d6, 150 MHz) δ 157.3, 134.0, 131.8, 127.4, 116.7, 116.6, 116.0, 72.1, 70.2, 67.6, 62.2; MS (MALDI-TOF) calcd for C₁₁H₁₄BrN₂O₄: 317.006; found: m/z 317.003 [M+H]⁺; calcd for C₁₁H₁₃BrN₂O₄Na: 339.006; found: m/z 339.006 [M+Na]⁺.

Sugar-5,6Br2BIMs:

### (1R,2S,3R)-1-(5,6-dibromo-1H-benzo[d]imidazole-2-yl)butane-1,2,3,4-tetraol

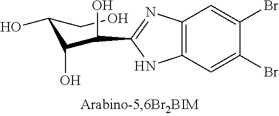

D-/L-arabinose (10 mg) and 4,5-dibromorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give AraBr2BIM. The supporting data is given below.

C ₁H₁₂Br₂N₂O₄; purple powder; mp=214-216° C.; [α]²⁵_{D}−25.3 (c 0.01, DMSO); ¹H NMR (DMSO-d6, 600 MHz) δ 7.87 (2H, s, ArH), 5.11 (1H, s, H1), 3.75 (1H, ddd, J=5.6, 3.2, 2.8 Hz, H3), 3.63 (1H, d, J=2.8 Hz, H2), 3.62 (1H, dd, J=10.7, 3.2 Hz, H4a), 3.44 (1H, dd, J=10.7, 5.6 Hz, H4b); ¹³C NMR (DMSO-d6, 150 MHz) δ 160.4, 139.3 (2×), 118.9 (2×), 115.2 (2×), 73.8, 70.9, 67.5, 63.4; MS (MALDI-TOF) calcd for C₁₁H₁₃Br₂N₂O₄: 394.916; found: m/z 394.921 [M+H]⁺.

### (1S,2R,3S,4R)-1-(5,6-dibromo-1H-benzo[d]imidazole-2-yl)pentane-1,2,3,4-tetraol

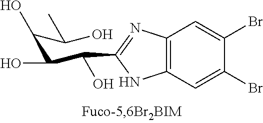

D-/L-fucose (10 mg) and 4,5-dibromorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give FucBr2BIM. The supporting data is given below.

C₁₂H₁₄Br₂N₂O₄; yellow-white powder; mp=184-186° C.; [α]²⁵_{D}+32.1 (c 0.01, DMSO); ¹H NMR (DMSO-d6, 600 MHz) δ 7.90 (2H, s, ArH), 5.16 (1H, s, H1), 4.25 (1H, d, J=6.3 Hz, H2), 3.92 (1H, dq, J=6.2, 5.8 Hz, H4), 3.88 (1H, dd, J=6.3, 5.8 Hz, H3), 1.11 (3H, d, J=6.2 Hz, H5); ¹³C NMR (DMSO-d6, 150 MHz) δ 160.6, 136.4, 136.3, 116.7, 115.7, 111.2, 111.1, 74.1, 72.8, 69.9, 68.2, 20.0; MS (MALDI-TOF) calcd for C₁₂H₁₅Br₂N₂O₄: 408.932; found: m/z 409.009 [M+H]⁺.

### (1S,2R,3S,4R)-1-(5,6-dibromo-1H-benzo[d]imidazol-2-yl)pentane-1,2,3,4,5-pentaol

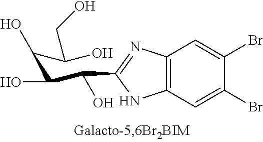

D-/L-galactose (10 mg) and 4,5-dibromorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GalBr2BIM. The supporting data is given below.

C₁₂H₁₄Br₂N₂O₅; white powder; mp=222-224° C.; [α]²⁵_{D}+12.4 (c 0.01, DMSO); ¹H NMR (DMSO-d6, 600 MHz) δ 7.91 (2H, s, ArH), 5.18 (1H, d, J=1.3 Hz, H1), 3.92 (1H, dd, J=9.4, 1.3 Hz, H2), 3.74 (1H, ddd, J=7.0, 6.2, 0.7 Hz, H4), 3.63 (1H, dd, J=9.4, 0.7 Hz, H3), 3.46 (1H, dd, J=10.5, 7.0 Hz, H5a), 3.43 (1H, dd, J=10.5, 6.2 Hz, H5b); ¹³C NMR (DMSO-d6, 150 MHz) δ 160.6, 138.0 (2×), 119.0 (2×), 116.2 (2×), 72.8, 69.8, 69.1, 67.6, 63.1; MS (MALDI-TOF) calcd for C₁₂H₁₅Br₂N₂O₅: 424.926; found: m/z 424.896 [M+H]⁺; calcd for C₁₂H₁₄Br₂N₂O₅Na: 446.926; found: m/z 446.870 [M+Na]⁺.

### (1S,2R,3S)-1-(5,6-dibromo-1H-benzo[d]imidazole-2-yl)-1,2,3,4-tetrtahydroxy pentanoic acid

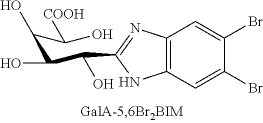

D-galacturonic acid (10 mg) and 4,5-dibromorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GalABr2BIM. The supporting data is given below.

C₁₂H₁₂Br₂N₂O₆; black powder; mp=132-134° C.; [α]²⁵_{D}+3.3 (c 0.01, DMSO); ¹H NMR (DMSO-d6, 600 MHz) δ 7.94 (2H, s, ArH), 5.18 (1H, d, J=0.7 Hz, H1), 4.30 (1H, d, J=0.9 Hz, H4), 3.98 (1H, dd, J=9.7, 0.9 Hz, H3), 3.93 (1H, dd, J=9.7, 0.7 Hz, H2); ¹³C NMR (DMSO-d6, 150 MHz) δ 175.4, 160.2, 136.9 (2×), 119.0 (2×), 116.8 (2×), 72.5, 71.4, 69.9, 67.2; MS (MALDI-TOF) calcd for C₁₂H₁₃Br₂N₂O₆: 438.906 [M+H]⁺.

### (1S,2R,3R,4R)-1-(5,6-dibromo-1H-benzo[d]imidazole-2-yl)pentane-1,2,3,4,5-pentaol

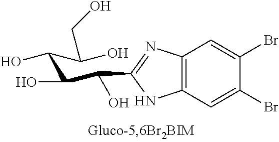

D-/L-glucose (10 mg) and 4,5-dibromorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GlcBr2BIM. The supporting data is given below.

C₁₂H₁₄Br₂N₂O₅; brownish powder; mp=168-170° C.; [α]²⁵_{D}−20.3 (c 0.005, DMSO); ¹H NMR (DMSO-d6, 600 MHz) δ 7.96 (2H, s, ArH), 5.02 (1H, d, J=5.5 Hz, H1), 4.11 (1H, dd, J=5.5, 1.0 Hz, H2), 3.74 (1H, dd, J=11.0, 3.2 Hz, H5a), 3.63 (1H, ddd, J=8.5, 5.6, 3.2 Hz, H4), 3.44 (1H, dd, J=8.5, 1.0 Hz, H3), 3.35 (1H, dd, J=11.0, 5.6 Hz, H5b); ¹³C NMR (DMSO-d6, 150 MHz) δ 161.3, 140.4 (2×), 128.3 (2×), 124.6 (2×), 74.2, 72.6, 71.1, 71.0, 63.3; MS (MALDI-TOF) calcd for C₁₂H₁₅Br₂N₂O₅: 424.927; found: m/z 424.913 [M+H]⁺; calcd for C₁₂H₁₄Br₂N₂O₅Na: 446.926; found: m/z 446.887 [M+Na]⁺

### (1S,2R,3R)-1-(5,6-dibromo-1H-benzo[d]imidazole-2-yl)-1,2,3,4-tetrtahydroxy pentanoic acid

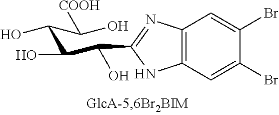

D-glucuronic acid (10 mg) and 4,5-dibromorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GlcABr2BIM. The supporting data is given below.

C₁₂H₁₂Br₂N₂O₆; gray powder; mp=140-142° C.; [α]²⁵_{D}+2.3 (c 0.01, DMSO); ¹H NMR (DMSO-d6, 600 MHz) δ 7.88 (2H, s, ArH), 4.92 (1H, d, J=5.7 Hz, H1), 3.97 (1H, dd, J=5.7, 1.3 Hz, H2), 3.96 (1H, d, J=7.3 Hz, H4), 3.60 (1H, dd, J=7.3, 1.3 Hz, H3); ¹³C NMR (DMSO-d6, 150 MHz) δ 174.8, 158.7, 138.2, 137.7, 119.3, 119.2, 116.6, 116.2, 71.8, 71.6, 70.1, 69.1; MS (MALDI-TOF) calcd for C₁₂H₁₃Br₂N₂O₆: 438.906 [M+H]⁺.

### N-((1S,2R,3R,4R)-1-(5,6-dibromo-1H-benzo[d]imidazol-2-yl)-2,3,4,5-tetrahydroxypentyl)-acetamide

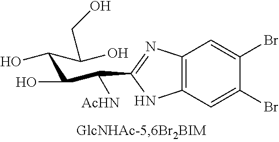

D-N-acetylglucosamine (10 mg) and 4,5-dibromorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give GlcNAcBr2BIM. The supporting data is given below.

C₁₄H₁₇Br₂N₃O₅; black powder; moisturized; [α]²⁵_{D}−6.3 (c 0.01, DMSO); ¹H NMR (DMSO-d6, 600 MHz) δ 8.06 (1H, s, ArH), 5.21 (1H, t, J=2.8 Hz, H1), 4.22 (1H, dd, J=6.0, 2.8 Hz, H2), 3.56 (1H, J=11.0, 3.3 Hz, H5a), 3.48 (1H, ddd, J=8.5, 5.5, 3.3 Hz, H4), 3.38 (1H, J=11.0, 5.5 Hz, H5b), 3.24 (1H, dd, J=8.5, 6.0 Hz, H3), 1.97 (3H, s, Me); ¹³C NMR (DMSO-d6, 150 MHz) δ 170.2, 156.4, 134.2 (2×), 119.5 (2×), 118.5 (2×), 71.2, 70.9, 70.3, 63.2, 51.6, 22.7; MS (MALDI-TOF) calcd for C₁₄H₁₈Br₂N₃O₅: 465.954 [M+H]⁺.

### (1R,2R,3R,4R)-1-(5,6-dibromo-1H-benzo[d]imidazole-2-yl)pentane-1,2,3,4,5-pentaol

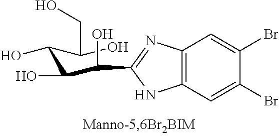

D-/L-mannose (10 mg) and 4,5-dibromorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give ManBr2BIM. The supporting data is given below.

C₁₂H₁₄Br₂N₂O₅; purple powder; mp=196-198° C.; [α]²⁵_{D}−22.0 (c 0.01, DMSO); ¹H NMR (DMSO-d6, 600 MHz) δ 7.95 (2H, s, ArH), 4.81 (1H, d, J=8.5 Hz, H1), 4.02 (1H, d, J=8.5 Hz, H2), 3.66 (1H, d, J=9.0 Hz, H3), 3.64 (1H, dd, J=10.9, 3.3 Hz, H5a), 3.51 (1H, ddd, J=9.0, 6.1, 3.3 Hz, H4), 3.43 (1H, dd, J=10.9, 6.1 Hz, H5b); ¹³C NMR (DMSO-d6, 150 MHz) δ 159.7, 137.4 (2×), 119.1 (2×), 116.5 (2×), 71.4, 71.0, 69.5, 67.7, 63.8; MS (MALDI-TOF) calcd for C₁₂H₁₅Br₂N₂O₅: 424.927; found: m/z 424.896 [M+H]⁺; calcd for C₁₂H₁₄Br₂N₂O₅Na: 446.926; found: m/z 446.884 [M+Na]⁺.

### (1R,2R,3R,4R)-1-(5,6-dibromo-1H-benzo[d]imidazole-2-yl)pentane-1,2,3,4-tetraol

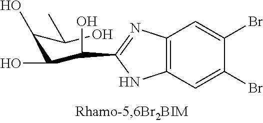

D-/L-rhamnose (10 mg) and 4,5-dibromorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give RhaBr2BIM. The supporting data is given below.

C₁₂H₁₄Br₂N₂O₄; red powder; mp=124-126° C.; [α]²⁵_{D}+37.3 (c 0.01, DMSO); ¹H NMR (DMSO-d6, 600 MHz) δ 8.15 (2H, s, ArH), 4.97 (1H, d, J=8.8 Hz, H1), 4.01 (1H, d, J=8.8 Hz, H2), 3.63 (1H, dq, J=8.6, 6.2 Hz, H4), 3.41 (1H, d, J=8.6 Hz, H3), 1.18 (3H, d, J=6.2 Hz, Me); ¹³C NMR (DMSO-d6, 150 MHz) δ 158.9, 132.1 (2×), 119.9 (2×), 118.7 (2×), 73.2, 71.2, 66.7, 65.7, 21.1; MS (MALDI-TOF) calcd for C₁₂H₁₅Br₂N₂O₄: 408.932; found: m/z 408.909 [M+H]⁺; calcd for C12H14Br₂N2O4Na: 430.932; found: m/z 430.898 [M+Na]⁺.

### (1S,2S,3R)-1-(5,6-dibromo-1H-benzo[d]imidazole-2-yl)butane-1,2,3,4-tetraol

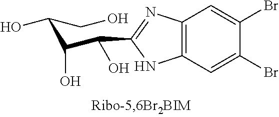

D-/L-ribose (10 mg) and 4,5-dibromorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give RibBr2BIM. The supporting data is given below.

C₁₁H₁₂Br₂N₂O₄; black powder; mp=244-246° C.; [α]²⁵_{D}+2.3 (c 0.01, DMSO); ¹H NMR (DMSO-d6, 600 MHz) δ 7.94 (1H, s, ArH), 5.07 (1H, d, J=4.4 Hz, H1), 3.82 (1H, dd, J=6.7, 4.4 Hz, H2), 3.55 (1H, dd, J=11.0, 3.5 Hz, H4a), 3.52 (1H, ddd, J=6.7, 5.8, 3.5 Hz, H3), 3.43 (1H, dd, J=11.0, 5.8 Hz, H4b); ¹³C NMR (DMSO-d6, 150 MHz) δ 158.9, 132.1 (2×), 119.9 (2×), 118.7 (2×), 74.7, 71.9, 69.1, 63.2; MS (MALDI-TOF) calcd for C₁₁H₁₃Br₂N₂O₄: 394.916 [M+H]⁺.

### (1S,2R,3R)-1-(5,6-dibromo-1H-benzo[d]imidazole-2-yl)butane-1,2,3,4-tetraol

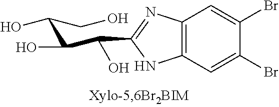

D-/L-xylose (10 mg) and 4,5-dibromorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give XylBr2BIM. The supporting data is given below.

C₁₁H₁₂Br₂N₂O₄; purple powder; mp=190-192° C.; [α]²⁵_{D}+3.3 (c 0.01, DMSO); ¹H NMR (DMSO-d6, 600 MHz) δ 8.06 (2H, s, ArH), 5.15 (1H, d, J=4.8 Hz, H1), 3.98 (1H, dd, J=4.8, 3.0 Hz, H2), 3.66 (1H, ddd, J=5.8, 4.6, 3.0 Hz, H3), 3.47 (1H, dd, J=10.7, 4.6 Hz, H4a), 3.38 (1H, dd, J=10.7, 5.8 Hz, H4b); ¹³C NMR (DMSO-d6, 150 MHz) δ 158.5, 132.3 (2×), 119.4 (2×), 118.5 (2×), 72.0, 70.0, 67.5, 62.1; MS (MALDI-TOF) calcd for C₁₁H₁₃Br₂N₂O₄: 394.916; found: m/z 394.876 [M+H]⁺; calcd for C₁₂H₁₄Br₂N₂O₅Na: 416.916; found: m/z 416.866 [M+Na]⁺.

### (1S,2R,3R,4R)-1-(5,6-dibromo-1H-benzo[d]imidazol-2-yl)-3-O-(2′,3′,4′,5′-tetrahydroxy-α-D-galactopyranosyl)pentane-1,2,4,5-tetraol

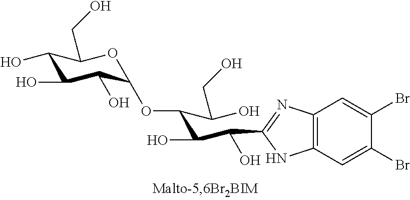

Maltose (10 mg) and 4,5-dibromorophenyldiamine (10 mg) with catalytic amount of iodine (1 mg) in acetic acid (1 mL) was reacted at room temperature for 12 hour. The resulting solution was precipitated by ethyl acetate (30 mL) and centrifuged for three times to remove the excess reagent. The pellet was lyophilized to give maltoBr2BIM. The supporting data is given below.

C₁₈H₂₄Br₂N₂O₁₀; black syrup; mp=158-160° C.; [α]²⁵_{D}−26.3 (c 0.01, DMSO); ¹H NMR (DMSO-d6, 600 MHz) δ 8.08 (2H, s, ArH), 5.30 (1H, d, J=4.9 Hz, H1′), 4.91 (1H, d, J=3.5 Hz, H1), 4.40 (1H, dd, J=5.1, 3.5 Hz, H2), 4.32 (1H, ddd, J=8.6, 5.0, 4.1 Hz, H4), 4.24 (1H, dd, J=8.6, 5.0 Hz, H3), 4.18 (1H, ddd, J=9.0, 4.6, 2.0 Hz, H5′), 3.80-3.70 (2H, m, H2′, H4′), 3.80 (1H, dd, J=12.2, 5.0 Hz, H5a), 3.75 (1H, dd, J=12.2, 4.1 Hz, H5b), 3.71 (1H, t, J=9.5 Hz, H3′), 3.51 (1H, dd, J=9.8, 3.8 Hz, H6a′), 3.35 (1H, t, J=9.8 Hz, H6b′); ¹³C NMR (DMSO-d6, 150 MHz) δ 157.7, 132.9 (2×), 119.5 (2×), 118.7 (2×), 103.8, 80.6, 75.5, 73.8, 72.5, 72.4, 71.5, 69.2, 67.9, 62.2, 61.0; MS (MALDI-TOF) calcd for C₁₈H₂₅Br₂N₂O₁₀: 586.980 [M+H]⁺.

### Example 2

### Analysis of Sugar-5FBIMs (Also Called SYBIM, Wherein Y=¹⁹F Isotope at C5 Position of BIM Ring) in ¹⁹F-NMR (470 MHz)

Aldoses (galactose, N-acetyl galactosamine, galactouronic acid, fucose, glucose, glucouronic acid, mannose, xylose, ribose, rhamnose, arabinose; 10 mg/each) and 4-fluorophenyldiamine (10 mg) with catalytic amount of iodine (1 mg) in acetic acid (1 mL) were mixed at room temperature for 12 hour to form a series of SFBIM (also called SYBIM, wherein Y=¹⁹F isotope) products. The resulting solution was precipitated by ethyl acetate (30 mL) and centrifuged for three times to remove the excess reagent. The pellets were lyophilized to give SFBIMs (˜15 mg/each). The SFBIM (1 mg) was dissolved in d-solvents (d-DMSO, d-MeOH or d-H₂O) for ¹⁹F NMR determination (data not shown). Based on the NMR results, various types of SFBIMs showed different chemical shift (also see table 1) so that these SFBIMs can be used for sugar compositional analysis by comparison of chemical shift in ¹⁹F NMR.

### Example 3

### Analysis for a Mixture Containing 9 Kinds of Sugar-5FBIMs and 2 Sugar-6FBQXs in ¹⁹F-NMR

The SFBIM (RibFBIM, GalFBIM, FucFBIM, GlcFBIM, RhaFBIM, XylFBIM, GalNAcFBIM, AraFBIM, GlcAFBIM; 0.5 mg/each) and SFBQX (SiaFBQX, KdoFBQX; 0.5 mg/each) were mixed in d-H₂O (or d-DMSO, d-MeOH) for ¹⁹F NMR determination (data not shown). Based on the NMR results, various typse of SFBIM and SFBQX showed different chemical shift (also see table 1) so that these SFBIMs and SFBQXs can be used for sugar compositional analysis by comparison of chemical shift in ¹⁹F NMR.

### Example 4

### Analysis for a Mixture Containing 12 Kinds of Sugar-5FBIMS on HPLC Spectrum with C18 Column

The SFBIM (RibFBIM, GalFBIM, FucFBIM, GlcFBIM, RhaFBIM, XylFBIM, GalNAcFBIM, AraFBIM, GlcAFBIM; 0.5 mg/each) and SFBQX (SiaFBQX, KdoFBQX; 0.5 mg/each) ware mixed in DMSO (or MeOH, H₂O) for HPLC analysis (data not shown). Based on the HPLC results, various types of SFBIM and SFBQX showed different retention times so that these SFBIMs, and SFBQXs can be used for sugar compositional analysis by comparison of retention time in HPLC system.

### Example 5

### Analysis for Sugar-5,6F₂BIMs (Also Called SYBIM, Wherein Y=¹⁹F Isotopes at C5 and C6 of BIM Ring) for Sugar Separation and Identification by HPLC

The mixture of sugar-5,6F₂BIMs was analysized by HPLC for sugar separation and identification. The SF₂BIM (RibF₂BIM, GalF₂BIM, FucF₂BIM, RhaF₂BIM, GlcNAcF₂BIM, ManF₂BIM, GlcAF₂BIM; 0.5 mg/each) and SFBQX (SiaF₂BQX, KdoF₂BQX; 0.5 mg/each) were mixed in DMSO (or MeOH, H₂O) for HPLC analysis (data not shown). Based on the HPLC results, various types of SFBIM and SFBQX showed different retention times so that these SF₂BIMs, and SF₂BQXs can be used for sugar compositional analysis by comparison of retention time in HPLC system.

### Example 6

### Detection Limitation of Maltohexose-YBIMs by MS, LC and LC/MS

MS

Maltohexose (10 mg) and 2,3-naphthelenediamine (10 mg) with catalytic amount of iodine (1 mg) in acetic acid (1 mL) was reacted at room temperature for 12 hour to form MaltohexoBBIM (also called MaltohexaoseNAIM). The resulting solution was precipitated by ethyl acetate (30 mL) and centrifuged for three times to remove the excess reagent. The pellet was lyophilized. The MaltohexoBBIM (0.1 mg) was dissolved in solvent for LC/MS determination. Based on the LC/MS results (data not shown), MaltohexoBBIM showed exact mass at 1151 Da and retention time at 21 min so that the SYBIMs can be identified and quantified for sugar determination by LC/MS.

Maltohexose (10 mg) and 4-fluorophenyldiamine (10 mg) with catalytic amount of iodine (1 mg) in acetic acid (1 mL) was mixed at room temperature for 12 hour to form MaltohexoFBIM. The resulting solution was precipitated by ethyl acetate (30 mL) and centrifuged for three times to remove the excess reagent. The pellet was lyophilized. The MaltohexoFBIM (0.1 mg) was dissolved in solvent for LC/MS determination. Based on the LC/MS results(data not shown), MaltohexoFBIM showed exact mass at 1119 Da and retention time at 12 min so that the SYBIMs can be identified and quantified for sugar determination by LC/MS.

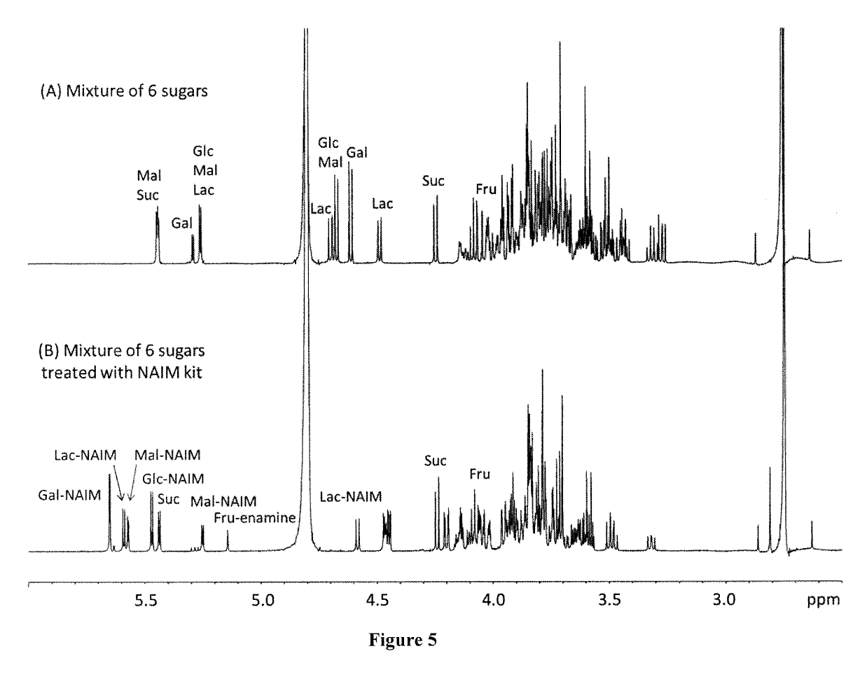

Maltohexose (10 mg) and DAB-Lys-FITC (10 mg) with catalytic amount of iodine (1 mg) in acetic acid (1 mL) was reacted at room temperature for 12 hour to form Maltohexo-Lys-FITC-BIM. The resulting solution was precipitated by ethyl acetate (30 mL) and centrifuged for three times to remove the excess reagent. The pellet was lyophilized. The Maltohexo-Lys-FITC-BIM (0.1 mg) was dissolved in solvent for LC/MS determination. Based on the LC/MS (see the lower in FIG. 5c), Maltohexo-Lys-FITC-BIM showed exact mass at 1624 Da and retention time at 51.9 min so that the SYBIMs can be identified and quantified for sugar determination by LC/MS.

LC

Maltohexose (10 mg) and 2,3-naphthelenediamine (10 mg) with catalytic amount of iodine (1 mg) in acetic acid (1 mL) was mixed at room temperature for 12 hour to form MaltohexoBBIM (same with MaltohexaoseNAIM). Maltohexose (10 mg) and 4-fluorophenyldiamine (10 mg) with catalytic amount of iodine (1 mg) in acetic acid (1 mL) was reacted at room temperature for 12 hour to form MaltohexoFBIM. The resulting solution was precipitated by ethyl acetate (30 mL) and centrifuged for three times to remove the excess reagent. The pellets were lyophilized to give MaltohexoBBIM and MaltohexoFBIM. The SYBIM (0.1 mg) was dissolved in solvent for HPLC determination. Based on the HPLC results (data not shown), MaltohexoBBIM showed the retention time at 11 min and MaltohexoFBIM showed the retention time at 4.3 min so that the SYBIMs can be identied and quantified for sugar determination by HPLC.

### Example 7

### Analysis for Monosaccharide Derivated SFBIMs with Chiral Shift Reagent for D-/L-Configuration (Enantiomeric Separation) Determination by ¹⁹F NMR Technology

D-/L-galactose (10 mg) and 4-fluorophenyldiamine (10 mg) with catalytic amount of iodine (1 mg) in acetic acid (1 mL) was mixed at room temperature for 12 hour to form GalFBIM. The D-/L-GalFBIM (0.5 mg) was dissolved in d-H₂O (or d-DMSO, d-MeOH) with catalytic amount of chiral shift reagent ((Eu(tfc)₃, 0.5 mg) for ¹⁹F NMR determination. Based on the NMR results (data not shown), various types of D-GalFBIM and L-GalFBIM showed different chemical shift so that these SFBIMs can be used for sugar configuration analysis by the variety of chemical shift in ¹⁹F NMR.

D-/L-fucose (10 mg) and 4-fluorophenyldiamine (10 mg) with catalytic amount of iodine (1 mg) in acetic acid (1 mL) was mixed at room temperature for 12 hour to form D-FucFBIM and L-FucFBIM. The D-/L-FucFBIM (0.5 mg) was dissolved in d-H₂O (or d-DMSO, d-MeOH) with catalytic amount of chiral shift reagent ((Eu(tfc)₃, 0.5 mg) for ¹⁹F NMR determination. Based on the NMR results (data not shown), various types of D-FucFBIM and L-FucFBIM showed different chemical shift so that these SFBIMs can be used for sugar configuration analysis by the variety of chemical shift in ¹⁹F NMR.

### Example 8

### Analysis for GlycanBBIM-Labeled N-Glycans by MS Spectra and Separation in HPLC Column

Fetuin (10 mg) was treated with trpsin and PNG-F to release N-glycan. Fetuin N-glycan (0.1 mg) and 2,3-naphthalenediamine (1 mg) with catalytic amount of iodine (1 mg) in acetic acid (1 mL) was reacted at room temperature for 12 hour to form fetuin N-glycanBBIMs. The resulting solution was precipitated by ethyl acetate (30 mL) and centrifuged for three times to remove the excess reagent. The pellet was lyophilized to give fetuin N-glycanBBIMs for MALDI-TOF MS and LC/MS analysis. The corresponding profile of annotated N-glycan-BBIMs from fetuin in HPLC and the LTQ-FTMS spectral profile were obtained (data not shown).

Ovalbumin N-glycan-BBIMs were determined by MALDI-TOF MS. Ovalbumin (10 mg) was treated with trpsin and PNG-F to release N-glycan. Ovalbumin N-glycan (0.1 mg) and 2,3-naphthalenediamine (1 mg) with catalytic amount of iodine (1 mg) in acetic acid (1 mL) was mixed at room temperature for 12 hour to form ovalbumin N-glycanBBIMs. The resulting solution was precipitated by ethyl acetate (30 mL) and centrifuged for three times to remove the excess reagent. The pellet was lyophilized to give the profile for ovalbumin N-glycanBBIMs by MALDI-TOF MS analysis (data not shown).

### Example 9

### Analysis for Per-Methylated SYBIMs (pSYBIMs) and the Nomenclature of Mass Fragment Ions

MaltohexoseBBIM (1 mg) was dissolved in DMSO (1 mL). The solution was added NaH (1 mg) and following the MeI (100 mg) was added. The permethylation reaction was completed at room temperature for 4 hour. After quenched and extracted the per-methylated maltohexoseBBIM was determined by MS for structural analysis. The fragment ions of SYBIM derivatives for structural analysis by MS and tandem MSⁿ (data not shown).

GlycanBBIMs (1 ug) from BIM derivatized ovalbumin N-glycan was dissolved in DMSO (1 mL). The solution was added NaH (1 mg) and following the MeI (100 mg) was added. The permethylation reaction was completed at room temperature for 4 hour. After quenched and extracted the per-methylated ovalbumin N-glycan BBIMs were measured by MS for structural analysis (data not shown).

GlycanYBIMs (ForssmanFBIM, GloboHFBIM, GD2FBIM, GD3FBIM, SSEA4FBIM, LeFBIM, GloboHBIM, ForssmanBIM, GD2BIM, GD3BIM, SSEA4BIM, 0.5 mg/each) were dissolved in DMSO (1 mL). The solution was added NaH (1 mg) and following the MeI (100 mg) was added. The permethylation reaction was completed at room temperature for 4 hour. After quenched and extracted, the per-methylated glycanFBIMs and glycanBlMs were determined by MS for structural analysis (data not shown). The possible mass fragments of SYBIMs and SYBQXs were determined (data not shown).

### Example 10

### Enzymatic Approach for the SYBIM Derivated Glycans Using for Glycan Sequencing

Various linkages of oligosaccharideBBlMs (maltohexoseBBIM, larminarihexoseBBIM, cellohexoseBBIM; 1 mg/each) were prepared by previous method. These glycanBBlMs can be degraded by special enzyme to know the real structures of glycan, for example, □-amylase, endo□-1,3-glucanase and cellulase, respectively. The results of enzymatic digestion of oligosaccharide-BBIMs by CE (data not shown).

Maltohexose (10 mg) and 4-fluorophenyldiamine (10 mg) with catalytic amount of iodine (1 mg) in acetic acid (1 mL) was mixed at room temperature for 12 hour to form MaltohexoFBIM. The resulting solution was precipitated by ethyl acetate (30 mL) and centrifuged for three times to remove the excess reagent. The pellet was lyophilized to give MaltohexoFBIM. The MaltohexoFBIM (1 mg) was dissolved in d-solvent for¹⁹F NMR determination. The MaltohexoFBIM can be a substract of enzymes, when treated with hydrolyase or transferase the LC, MS, and NMR signals will be changed. Based on the results of SYBIMs (data not shown), the Maltohexose-5FBIM as glycan tagging product can be used for enzyme screening, structural identification and quantification of glycan.

### Example 11

### Preparation Method of New Glycopeptides/Glycoproteins

DAB-peptides (20 mg/each) were obtained by solid phase synthesizer. The DAB linker was set at Asn (N-glycoprotein) or Thr/Ser (O-glycoprotein). The glycan (1 mg/each) and DAB-peptides (2 mg) with catalytic amount of iodine (1 mg) in acetic acid (1 mL) was mixed at room temperature for 12 hour to form N-Glycan-peptide-BIMs and O-Glycan-peptide-BIMs as new types of glycopeptides/glycoproteins. The resulting solution was precipitated by ethyl acetate (10 mL) and centrifuged for three times to remove the excess reagent. The pellets were lyophilized to give N-Glycan-peptide-BIMs and O-Glycan-peptide-BIMs. These new glycopeptides/glycoproteins were measured by LC/MS determination and testing with biological assay so that these new glycopeptides/glycoproteins can be used for structural determination of glycoproteins and functional assay.

### Example 12

### Automatic Glycan Sequencing Using SYBIMs or pSYBIMs

A scheme of the method for determining the sequence of a glycan is given in FIG. 2. For example, MaltohexoseBBIM was prepared by mixed of maltohexose (10 mg) and 2,3-naphthalenediamine (10 mg) with catalytic amount of iodine (1 mg) in acetic acid (1 mL) at room temperature for 12 hour. The MaltohexoseBBIM (6 mg) was partial hydrolysis by acidic solution to form the GlcBBIM (no. 1 sugar) and maltopentose. The maltopentose was mixed with 4-fluorophenyldiamine (1 mg) with catalytic amount of iodine (1 mg) in acetic acid (1 mL) at room temperature for 12 hour to form the MaltopentoseFBIM and then the residue was further partial hydrolysis by acidic solution to form the GlcFBIM (no. 2 sugar) and maltotetraose. Following the stepwise tagging and degradation process the GlcF₂BIM (no. 3 sugar), GlcClBIM (no. 4 sugar), GlcCl₂BIM (no. 5 sugar), GlcBrBIM (no. 6 sugar) were obtained for NMR, HPLC, LC/MS analysis to identify the sequence of glycan. The reaction can be finished by one pot process (multiple reactions in one flask) before analyze and identify the sequence of glycan by comparison of SYBIMs or through the one by one LC/MS, LC/NMR to identify the sequence of glycan by measurement of SYBIMs. This glycan sequencing method can be extended for an automatic glycan sequencer (continuing tagging/degradation and analysis to speed the structural identification of glycan).

### Example 13

### Analysis of Sugar-5FBIMs by ¹⁹F NMR

A sugar-5FBIMs (using the new isotope-labelled compounds) were analysized in ¹⁹F NMR^{a}. The results were shown in Table 1.

TABLE 1
Chemical shifts of sugar-5FBIMs in ¹⁹F NMR^{a} at 298 °K

| | Solvent | Solvent | |
|---|---|---|---|
| Sugar-5FBIM | (D₂O)^{b} | (CD₃OD)^{b} | Solvent (DMSO-d6)^{b} |
| Arabinose | −118.06 | −122.31 | −124.40 |
| Fucose | −116.89 | −121.97 | −124.61 |
| Galactose | −117.45 | −117.75 | −119.76 |
| Galacturonic acid | −116.81 | −121.07 | −123.43 |
| N-acetyl | −117.20 | −117.85 | −119.92 |
| galactosamine | | | |
| Galactosamine | −116.88 | −122.80 | −125.82 |
| Glucose | −116.50 | −116.71 | −119.02 |
| Glucuronic acid | −116.20 | −120.95 | −124.37 |
| N-acetyl glucosamine | −116.64 | −116.36 | −119.43 |
| Glucosamine | −116.06 | −122.79 | −125.60 |
| Mannose | −119.59 | −121.29 | −123.94 |
| N-acetyl | −115.30^{c} | −115.53^{c} | — |
| mannosamine | | | |
| Maltose | −117.68 | −117.82 | −119.42 |
| Maltotriose | −117.86 | −118.13 | −120.25 |
| Maltohexose | −117.00 | −116.48 | −119.01 |
| Rhamnose | −117.70 | −116.77 | −119.47 |
| Ribose | −116.01 | −115.80 | −118.16 |
| Xylose | −116.52 | −117.03 | −118.99 |

^{a}470 MHz ¹⁹F NMR was used.
^{b}Trifluorotoulene was used as an internal standard at −63.72 ppm.
^{c}Trifluoroacetic acid was used as an internal standard at −76.55 ppm.

### Example 14

### Analysis of Sugar-5,6F₂BIMs by ¹⁹F NMR

A sugar-5,6F₂BIMs (using the new isotope-labeled compounds) were analysized in ¹⁹F NMR^{a}. The results were shown in Table 2

TABLE 2
Chemical shifts of sugar-5,6F₂BIMs in ¹⁹F NMR^{a}

| Sugar-5,6F₂BIM | Solvent (D₂O)^{b} | Solvent (CD₃OD)^{b,c} |
|---|---|---|
| Arabinose | −142.92 | −140.23^{c} |
| Fucose | −140.04 | −140.14^{c} |
| Galactose | −140.48 | −143.63^{b} |
| Galacturonic acid | −139.62 | −144.15^{b} |
| N-acetyl galactosamine | −139.90/−141.05 | −140.84^{b}/−142.72^{b} |
| Galactosamine | −143.57 | −144.53/−137.15^{d} |
| Glucose | −141.77 | −143.80^{b} |
| Glucuronic acid | −142.87 | −145.56^{b} |
| N-acetyl glucosamine | −141.19 | −143.04^{b} |
| Glucosamine | −144.70 | −138.15^{d} |
| Mannose | −138.91 | −138.71^{c} |
| N-acetyl mannosamine | −141.24 | −140.64^{c} |
| Maltose | −141.35 | −139.82^{c} |
| Maltotriose | −140.32 | −139.44^{c} |
| Maltohexose | −140.06 | — |
| Rhamnose | −139.54 | −143.04^{b} |
| Ribose | −139.06 | −140.53^{b} |
| Xylose | −139.27 | −140.31^{b} |

^{a}470 MHz ¹⁹F NMR was used.
^{b}Trifluorotoulene was used as an internal standard at −63.72 ppm.
^{c}Trifluoroacetic acid was used as an internal standard at −76.55 ppm.
^{d}DMSO-d₆ as solvent.

### Example 15

### Analysis of Sugar-5CF₃BIMs by ¹⁹F NMR

A sugar-5CF₃BIMs (using the new isotope-labelled compounds) were analysized in ¹⁹F NMR^{a}. The results were shown in Table 3.

TABLE 3
Chemical shifts of sugar-5CF₃BIMs in ¹⁹F NMR^{a}

| Sugar-5CF₃BIM | Solvent (D₂O)^{b} | Solvent (CD₃OD)^{b} |
|---|---|---|
| Arabinose | −61.76 | — |
| Fucose | −62.11 | −61.60 (DMSO) |
| Galactose | −61.86 | −61.64 (DMSO) |
| Galacturonic acid | −62.16 | −61.31 (DMSO) |
| N-acetyl galactosamine | −61.74 | −61.13 (DMSO) |
| Glucose | −62.24 | −61.71 |
| Glucuronic acid | −63.50 | −62.04 |
| N-acetyl glucosamine | −62.32 | −61.84 |
| Mannose | −61.90 | −61.54 (DMSO) |
| N-acetyl mannosamine | — | |
| Ribose | −62.27 | −61.76 |
| Xylose | −62.26 | −61.78 |

^{a}470 MHz ¹⁹F NMR was used.
^{b}Trifluoroacetic acid was used as an internal standard at −76.55 ppm.

### Example 16

### Analysis of Sugar-FBQXs in ¹⁹F NMR

A sugar-FBQXs (using the new isotope-labeled compounds) were analysized in ¹⁹F NMR^{a}. The results were shown in Table 4.

TABLE 4
Chemical shifts of sugar-FBQXs in ¹⁹F NMR^{a}

| | Solvent (D₂O)^{b} | Solvent (CD₃OD)^{b} |
|---|---|---|
| Sugar-5FBQX | | |
| Neu5Ac | −109.25 | −111.00/−111.00 |
| Neu5Gc | −109.21 | −110.98 |
| KDN | −109.36 | −111.04p |
| KDO | −109.84 | −110.92/113.38 (DMSO-d) |
| Sugar-5,6F₂BQX | | |
| Neu5Ac | −132.36 | — |
| Neu5Gc | −133.00 | −134.61/−137.03 (DMSO) |
| KDN | −136.78 | −137.00 |
| KDO | −133.37 | — |
| Sugar-5CF₃BQX | | |
| Neu5Ac | −62.72 | −63.06/−61.78 (DMSO) |
| Neu5Gc | −62.73 | −63.06/−61.94 (DMSO) |
| KDN | −62.73 | −63.07/−61.84 (DMSO) |
| KDO | −62.72 | −63.07/−61.79 (DMSO) |

^{a}470 MHz ¹⁹F NMR was used.
^{b}Trifluoroacetic acid was used as an internal standard at −76.55 ppm.

### Example 17

### Analysis of Sugar-FBHZs in ¹⁹F NMR

A sugar-FBHZs (using the new isotope-labelled compounds) were analysized in ¹⁹F NMR^{a}. The results were shown in Table 5.

TABLE 5
Chemical shifts of sugar-FBHZs in ¹⁹F NMR^{a}

| | Solvent (D₂O)^{b} | Solvent (CD₃OD)^{b} |
|---|---|---|
| Sugar-4FBHZ | | |
| Fructose | −128.18 | −126.69/−128.18 (DMSO) |
| Sorbose | −128.24 | −126.82/−128.24 (DMSO) |
| Sugar-3,5F₂BHZ | | |
| Fructose | −111.18 | −111.30/−112.35 (DMSO) |
| Sorbose | −112.19 | −111.78/−112.19 (DMSO) |

^{a}470 MHz ¹⁹F NMR was used.
^{b}Trifluorotoluene was used as an internal standard at −63.72 ppm.

### Example 18

### Analysis for a Composition of Glycans by ¹⁹F-NMR and HPLC

18.1 Analysis for Polysaccharides of Ganoderma lucidum (FWS fraction):

Polysaccharides of Ganoderma lucidum (FWS fraction) were analysized by the method according to the invention.

Sugar-5FBIMs labeled: ¹⁹F-NMR (D2O; CD₃OD, 470 MHz):

The results of the composition analysis of FWS (polysaccharides of G. lucidum) in ¹⁹F NMR^{a} using Sugar-5FBIMs labeled are given in Table 6 below.

TABLE 6
Composition analysis of FWS (polysaccharides of G. lucidum)
in ¹⁹F NMR^{a}

| Sugar- | Chemical shift | Percentage | Chemical shift | Percentage |
|---|---|---|---|---|
| 5FBIM | (in D₂O)^{b} | (integration) | (in CD₃OD)^{b} | (integration) |
| Glucose | −116.23 | 41 (3.92) | −116.30 | 46 (5.62) |
| Fucose | −115.93 | 25 (2.45) | −115.60 | 22 (2.63) |
| Mannose | −116.03 | 21 (2.03) | −115.85 | 20 (2.46) |
| Galactose | −116.11 | 10 (1.00) | −116.28 | 8 (1.00) |
| Xylose | −115.71 | 3 (0.3) | −116.01 | 4 (0.50) |

^{a}470 MHz ¹⁹F NMR was used.
^{b}Trifluorotoulene was used as an internal standard at −63.72 ppm.

The results of the composition analysis of FWS (polysaccharides of G. lucidum) using Sugar-5FBIMs labeled: 19F by HPLC are given in Table below.

TABLE 7
Composition analysis of FWS (polysaccharides of G. lucidum) by HPLC

| Sugar-5FBIM | Retention time (min) | Percentage |
|---|---|---|
| Glucose | 11.2 | 50 |
| Fucose | 12.5 | 20 |
| Mannose | 10.4 | 16 |
| Galactose | 13.5 | 10 |
| Xylose | 14.5 | 4 |

Sugar-5,6F₂BIMs labeled: ¹⁹F-NMR (D2O; CD₃OD, 470 MHz):

The results of the composition analysis of FWS (polysaccharides of G. lucidum) in ¹⁹F NMR^{a} using Sugar-5,6F₂BIMs labeled: ¹⁹F are given in Table 8 below.

TABLE 8
Composition analysis of FWS (polysaccharides of G. lucidum) by
¹⁹F NMR^{a}

| Sugar- | Chemical shift | Percentage | Chemical shift | Percentage |
|---|---|---|---|---|
| 5,6F₂BIM | (in D₂O)^{b} | (integration) | (in CD₃OD)^{b} | (integration) |
| Glucose | −138.91 | 49 (5.42) | −139.59 | 50 (3.50) |
| Fucose | −138.70 | 19 (2.00) | −138.80 | 18 (1.27) |
| Mannose | −138.51 | 16 (1.80) | −139.30 | 14 (1.00) |
| Galactose | −138.80 | 10 (1.15) | −139.53 | 11 (0.80) |
| Xylose | −138.24 | 6 (0.73) | −139.40 | 7 (0.40) |

^{a}470 MHz ¹⁹F NMR was used.
^{b}Trifluorotoulene was used as an internal standard at −63.72 ppm.

The composition analysis of FWS (polysaccharides of G. lucidum) by HPLC using Sugar-5,6F₂BIMs labeled: ¹⁹F are given in Table 9.

TABLE 9
Composition analysis of FWS (polysaccharides of G. lucidum) by HPLC

| Sugar-5,6F₂BIM | Retention time (min) | Percentage |
|---|---|---|
| Glucose | 17.0 | 50 |
| Fucose | 19.2 | 20 |
| Mannose | 16.1 | 16 |
| Galactose | 17.3 | 10 |
| Xylose | 21.2 | 4 |

18.2 Analysis for Xyloglucan:

Xyloglucan; Glc₄Xyl₃Gal₂

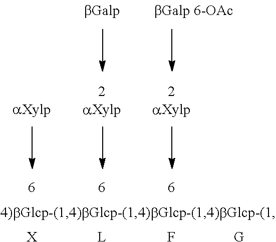

Xyloglucan was analysized by the method according to the invention.

Sugar-5FBIMs labeled: ¹⁹F-NMR (D2O; CD₃OD, 470 MHz):

The results of the composition analysis of xyloglucan (Glc₄Xyl₃Gal₂) in ¹⁹F NMR^{a} using Sugar-5FBIMs labeled: ¹⁹F are given in Table 10 below.

TABLE 10
Composition analysis of xyloglucan (Glc₄Xyl₃Gal₂) by ¹⁹F NMR^{a}

| Sugar- | Chemical shift | Percentage | Chemical shift | Percentage |
|---|---|---|---|---|
| 5FBIM | (in D₂O)^{b} | (integration) | (in CD₃OD)^{b} | (integration) |
| Glucose | −116.45 | 44 (4.06) | −116.23 | 44 (4.11) |
| Xylose | −116.26 | 34 (3.17) | −116.15 | 34 (3.11) |
| Galactose | −116.38 | 22 (2.00) | −115.74 | 22 (2.00) |

^{a}470 MHz ¹⁹F NMR was used.
^{b}Trifluorotoulene was used as an internal standard

at −63.72 ppm.

The results of the composition analysis of xyloglucan (Glc₄Xyl₃Gal₂) by HPLC using Sugar-5FBIMs labeled: ¹⁹F are given in Table 11 below.

TABLE 11
Composition analysis of xyloglucan (Glc₄Xyl₃Gal₂) by HPLC

| Sugar-5FBIM | Retention time (min) | Percentage |
|---|---|---|
| Glucose | 11.2 | 44 |
| Xylose | 12.6 | 33 |
| Galactose | 11.2 | 23 |

Sugar-5,6F₂BIMs labeled: ¹⁹F-NMR (D2O; CD₃OD, 470 MHz)

The results of the composition analysis of xyloglucan (Glc₄Xyl₃Gal₂) by ¹⁹F NMR^{a} using Sugar-5,6F₂BIMs labeled: ¹⁹F are given in Table 12 below.

TABLE 12
Composition analysis of xyloglucan (Glc₄Xyl₃Gal₂) by ¹⁹F NMR^{a}

| Sugar- | Chemical shift | Percentage | Chemical shift | Percentage |
|---|---|---|---|---|
| 5,6F₂BIM | (in D₂O)^{b} | (integration) | (in CD₃OD)^{b} | (integration) |
| Glucose | −139.24 | 45 (1.41) | −140.11 | 44 (—) |
| Xylose | −139.05 | 32 (1.00) | −140.11 | 33 (—) |
| Galactose | −139.24 | 23 (0.71) | −140.11 | (—) |

^{a}470 MHz ¹⁹F NMR was used.
^{b}Trifluorotoulene was used as an internal standard at −63.72 ppm.

The results of the composition analysis of xyloglucan (Glc₄Xyl₃Gal₂) by HPLC using Sugar-5,6F₂BIMs labeled: ¹⁹F are given in Table 13 below.

TABLE 13
Composition analysis of xyloglucan (Glc₄Xyl₃Gal₂) by HPLC using
Sugar-5,6F₂BIMs labeled. ¹⁹F

| Sugar-5,6F₂BIM | Retention time (min) | Percentage |
|---|---|---|
| Glucose | 16.5 | 44 |
| Xylose | 17.2 | 33 |
| Galactose | 19.2 | 23 |

18.3 Analysis for LPS (E. coli. 055:B5, Sigma 034k4112)

Sugar-5FBIMs labeled: ·F-NMR (D2O; CD₃OD, 470 MHz):

The results of the composition analysis of LPS (E. coli. 055:B5) by ¹⁹F NMR^{a} using Sugar-5FBIMs labeled: ¹⁹F are given in Table 14 below.

TABLE 14
Composition analysis of LPS (E. coli. 055:B5) by ¹⁹F NMR^{a}

| Sugar- | Chemical shift | Percentage | Chemical shift | Percentage |
|---|---|---|---|---|
| 5FBIM | (in D₂O)^{b} | (integration) | (in CD₃OD)^{b} | (integration) |
| Rhamnose | −114.54 | 28 (7.72) | −115.76 | 28 (3.4) |
| Fucose | −115.56 | 6 (1.69) | −116.00 | 8 (0.9) |
| Galactose | −115.91 | 14 (3.71) | −116.40 | 14 (1.7) |
| Glucose | −116.02 | 33 (9.10) | −116.43 | 38 (4.5) |
| GlcNHAc | −116.23 | 8 (2.21) | −116.66 | 8 (1.0) |
| GalNHAc | −116.77 | 4 (1.00) | −117.60 | 4 (0.5) |
| KDO | −109.49 | 7 (1.79) | −108.50 | —(—) |

^{a}470 MHz ¹⁹F NMR was used.
^{b}Trifluorotoulene was used as an internal standard at −63.72 ppm.

The results of the composition analysis of LPS (E. coli. 055:B5) by HPLC using Sugar-5FBIMs labeled: ¹⁹F are given in Table 15 below.

TABLE 15
Composition analysis of LPS (E. coli. 055:B5) by HPLC

| Sugar-5FBIM | Retention time (min) | Percentage |
|---|---|---|
| Rhamnose | 12.0 | 18 |
| Fucose | 11.9 | 6 |
| Galactose | 11.8 | 14 |
| Glucose | 11.7 | 33 |
| GlcNHAc | 25.1 | 18 |
| GalNHAc | 22.8 | 4 |
| KDO | 8.0 | 7 |

Sugar-5,6F₂BIMs labeled: ¹⁹F-NMR (D2O; CD₃OD, 470 MHz):

The results of the composition analysis of LPS (E. coli. 055:B5) by ¹⁹F NMR^{a} using Sugar-5,6F₂BIMs labeled: ¹⁹F are given in Table 16 below.

TABLE 16
Composition analysis of LPS (E. coli. 055:B5) by ¹⁹F NMR^{a}

| | Chemical | | Chemical | |
|---|---|---|---|---|
| | shift | Percentage | shift | Percentage |
| Sugar-5,6F₂BIM | (in D₂O)^{b} | (integration) | (in CD₃OD)^{b} | (integration) |
| Rhamnose | −138.52 | 25 (1.50) | −139.02 | 25 (1.36) |
| Fucose | −138.38 | 11 (0.64) | −138.70 | 9 (0.50) |
| Galactose | −138.75 | 12 (0.72) | −138.98 | 11 (0.60) |
| Glucose | −138.52 | 30 (1.75) | −139.01 | 30 (1.66) |
| GlcNHAc | −139.39 | 3 (0.18) | −139.03 | 4 (0.24) |
| GalNHAc | −137.71 | 2 (0.12) | −139.02 | 3 (0.16) |
| KDO | −131.15 | 17 (1.00) | −132.93 | 18 (1.00) |

^{a}470 MHz ¹⁹F NMR was used.
^{b}Trifluorotoulene was used as an internal standard at −63.72 ppm.

The results of the composition analysis of LPS (E. coli. 055:B5) by HPLC using Sugar-5,6F₂BIMs labeled: ¹⁹F are given in Table 17 below.

TABLE 17
Composition analysis of LPS (E. coli. 055:B5) by HPLC

| Sugar-5,6F₂BIM | Retention time (min) | Percentage |
|---|---|---|
| Rhamnose | 14.3 | 19 |
| Fucose | 14.5 | 6 |
| Galactose | 17.9 | 14 |
| Glucose | 17.8 | 43 |
| GlcNHAc | 23.3 | 5 |
| GalNHAc | 22.2 | 4 |
| KDO | 11.0 | 9 |

18.4 Analysis for GM3; 3′sialyllactose; Neu5Acα2-3Galβ1-4Glc; ganglioside sugar

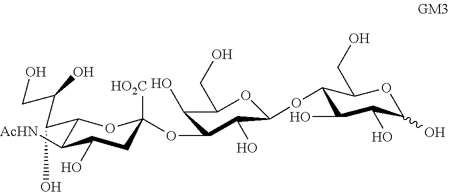

Sugar-5FBIMs labeled: ¹⁹F-NMR (D20; CD₃OD, 470 MHz):

The results of the composition analysis of GM3 (Neu5Acα2-3Galβ1-4Glc) by ¹⁹F NMR^{a} using Sugar-5FBIMs labeled: ¹⁹F are given in Table 18 below.

TABLE 18
Composition analysis of GM3 (Neu5Acα2-3Galβ1-4Glc)
by ¹⁹F NMR^{a}

| | Chemical | | Chemical | |
|---|---|---|---|---|
| | shift | Percentage | shift | Percentage |
| Sugar-5FBIM | (in D₂O)^{b} | (integration) | (in CD₃OD)^{b} | (integration) |
| Glucose | −116.35 | 42 (4.11) | −116.37 | 43 (3.4) |
| Galactose | −116.15 | 47 (4.70) | −115.96 | 44 (3.5) |
| Sialic acid | −109.51 | 11 (1.00) | −110.03 | (1.0) |

^{a}470 MHz ¹⁹F NMR was used.
^{b}Trifluorotoulene was used as an internal standard at −63.72 ppm.

The results of the composition analysis of GM3 (Neu5Acα2-3Galβ1-4Glc) by HPLC using Sugar-5FBIMs labeled: ¹⁹F were given in Table 19 below.

TABLE 19
Composition analysis of GM3 (Neu5Acα2-3Galβ1-4Glc) by HPLC

| Sugar-5FBIM | Retention time (min) | Percentage |
|---|---|---|
| Glucose | 11.2 | 44 |
| Galactose | 11.2 | 43 |
| Sialic acid | 9.2 | 13 |

Sugar-5,6F₂BIMs labeled: ¹⁹ F-NMR (D2O; CD₃OD, 470 MHz):

The results of the composition analysis of GM3 (Neu5Acα2-3Galβ1-4Glc) by ¹⁹F NMR^{a} using Sugar-5,6F₂BIMs labeled: ¹⁹F are given in Table 20 below.

TABLE 20
Composition analysis of GM3 (Neu5Acα2-3Galβ1-4Glc)
by ¹⁹F NMR^{a}

| | Chemical | | Chemical | |
|---|---|---|---|---|
| | shift | Percentage | shift | Percentage |
| Sugar-5,6F₂BIM | (in D₂O)^{b} | (integration) | (in CD₃OD)^{b} | (integration) |
| Glucose | −139.44 | 39 (2.0) | −139.71 | 40 (2.1) |
| Galactose | −139.26 | 41 (2.1) | −139.70 | 41 (2.2) |
| Sialic acid | −133.07 | 20 (1.0) | −134.90 | 19 (1.0) |

^{a}470 MHz ¹⁹F NMR was used.
^{b}Trifluorotoulene was used as an internal standard at −63.72 ppm.

The results of the composition analysis of GM3 (Neu5Acα2-3Galβ1-4Glc) by HPLC using Sugar-5,6F₂BIMs labeled: ¹⁹F are given in Table 21 below.

TABLE 21
Composition analysis of GM3 (Neu5Acα2-3Galβ1-4Glc)
by HPLC

| Sugar-5,6F₂BIM | Retention time (min) | Percentage |
|---|---|---|
| Glucose | 17.0 | 40 |
| Galactose | 17.5 | 40 |
| Sialic acid | 15.1 | 20 |

18.5 Analysis for Globopentose (Gb5); Galβ1-3GalNAβ1-3Galα1-4Galβ1-4Glc; Stage Specific Embryonic Antigen 3 (SSEA-3)

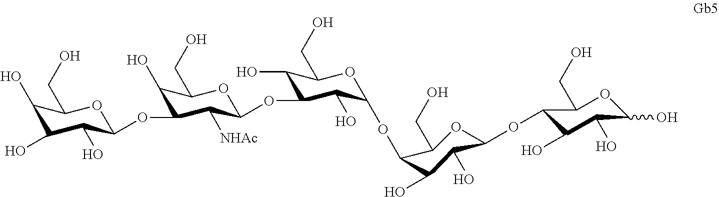

Sugar-5FBIMs labeled: ¹⁹F-NMR (D2O; CD₃OD, 470 MHz):

The results of the composition analysis of Gb5 (Galβ1-3GalNAcβ1-3Galα1-4Galβ1-4Glc) by ¹⁹F-NMR using Sugar-5FBIMs labeled: ¹⁹F are given in Table 22 below.

TABLE 22
Composition analysis of Gb5 (Galβ1-3GalNAcβ1-3Galα1-
4Galβ1-4Glc) by ¹⁹F-NMR^{a}

| | Chemical | | Chemical | |
|---|---|---|---|---|
| | shift | Percentage | shift | Percentage |
| Sugar-5FBIM | (in D₂O)^{b} | (integration) | (in CD₃OD)^{b} | (integration) |
| Glucose | −116.12 | 39 (5.20) | −115.79 | 40 (1.83) |
| Galactose | −116.10 | 38 (5.14) | −115.70 | 39 (1.75) |
| Gal-NHAc | −116.31 | 23 (3.15) | −116.27 | 21 (1.00) |

^{a}470 MHz ¹⁹F NMR was used.
^{b}Trifluorotoulene was used as an internal standard at −63.72 ppm.

The results of the composition analysis of Gb5 (Galβ1-3GalNAcβ1-3Galα1-4Galβ1-4Glc) by HPLC using Sugar-5FBIMs labeled: ¹⁹F are given in Table 23 below.

TABLE 23
Composition analysis of Gb5 (Galβ1-3GalNAcβ1-
3Galα1-4Galβ1-4Glc) by HPLC

| Sugar-5FBIM | Retention time (min) | Percentage |
|---|---|---|
| Glucose | 11.1 | 40 |
| Galactose | 11.2 | 40 |
| Gal-NHAc | 23.0 | 20 |

Sugar-5,6F₂BIMs labeled: ¹⁹F-NMR (D2O; CD₃OD, 470 MHz):

The results of the composition analysis of Gb5 (Galβ1-3GalNAcβ1-3Galα1-4Galβ1-4Glc) by ¹⁹F-NMR using Sugar-5,6F₂BIMs labeled: ¹⁹F are given in Table 24 below.

TABLE 24
Composition analysis of Gb5 (Galβ1-3GalNAcβ1-
3Galα1-4Galβ1-4Glc)

| | Chemical | | Chemical | |
|---|---|---|---|---|
| | shift | Percentage | shift | Percentage |
| Sugar-5,6F₂BIM | (in D₂O)^{b} | (integration) | (in CD₃OD)^{b} | (integration) |
| Glucose | −139.41 | 40 (0.51) | −139.11 | 39 (1.60) |
| Galactose | −139.40 | 39 (0.49) | −139.10 | 37 (1.50) |
| Gal-NHAc | −139.60 | 21 (0.28) | −139.48 | 24 (1.00) |

^{a}470 MHz ¹⁹F NMR was used.
^{b}Trifluorotoulene was used as an internal standard at −63.72 ppm.

The results of the composition analysis of Gb5 (Galβ1-3GalNAcβ1-3Galα1-4Galβ1-4Glc) by HPLC using Sugar-5,6F₂BIMs labeled: ¹⁹F are given in Table 25 below.

TABLE 25
Composition analysis of Gb5 (Galβ1-3GalNAcβ1-
3Galα1-4Galβ1-4Glc) by HPLC

| Sugar-5,6F₂BIM | Retention time (min) | Percentage |
|---|---|---|
| Glucose | 17.4 | 40 |
| Galactose | 17.5 | 40 |
| Gal-NHAc | 16.8 | 20 |

18.6 Analysis for Lewis^{Y} pentaose; Fucα1-2Galβ1-4(fucα1-3)GlcNAcβ1-3Gal; Lewis antigen

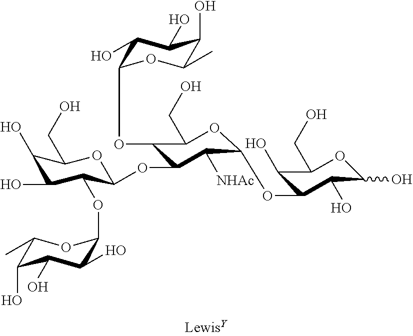

Sugar-5FBIMs labeled: ¹⁹F-NMR (D20; CD₃OD, 470 MHz):

The results of the composition analysis of Lewis^{Y} (Fucα1-2Galβ1-4(fucα1-3)GlcNAcβ1-3Gal) by ¹⁹F-NMR using Sugar-5FBIMs labeled: ¹⁹F are given in Table 26 below.

TABLE 26
Composition analysis of Lewis^{Y} (Fucα1-2Galβ1-
4(fucα1-3)GlcNAcβ1-3Gal) by ¹⁹F-NMR^{a}

| | Chemical | | Chemical | |
|---|---|---|---|---|
| | shift | Percentage | shift | Percentage |
| Sugar-5FBIM | (in D₂O)^{b} | (integration) | (in CD₃OD)^{b} | (integration) |
| Fucose | −116.11 | 40 (2.00) | −115.99 | 40 (2.00) |
| Galactose | −116.11 | 40 (2.00) | −115.99 | 40 (2.00) |
| Glc-NHAc | −116.11 | 20 (1.00) | −115.99 | 20 (1.00) |

^{a}470 MHz ¹⁹F NMR was used.
^{b}Trifluorotoulene was used as an internal standard at −63.72 ppm.

The results of the composition analysis of Lewis^{Y} (Fucα1-2Galβ1-4(fucα1-3)GlcNAcβ1-3Gal) by HPLC using Sugar-5FBIMs labeled: ¹⁹F are given in Table 27 below.

TABLE 27
Composition of Lewis^{Y} (Fucα1-2Galβ1-
4(fucα1-3)GlcNAcβ1-3Gal) by HPLC

| Sugar-5FBIM | Retention time (min) | Percentage |
|---|---|---|
| Fucose | 13.8 | 40 |
| Galactose | 11.8 | 40 |
| Glc-NHAc | 25.1 | 20 |

Sugar-5,6F₂BIMs labeled: ¹⁹F-NMR (D2O; CD₃OD, 470 MHz):

The results of the composition analysis of Lewis^{Y} (Fucα1-2Galβ1-4(fucα1-3)GlcNAcβ1-3Gal) by ¹⁹F-NMR using Sugar-5,6F₂BIMs labeled: ¹⁹F are given in Table 28 below.

TABLE 28
Composition analysis of Lewis^{Y} (Fucα1-2Galβ1-
4(fucα1-3)GlcNAcβ1-3Gal) by ¹⁹F-NMR^{a}

| | Chemical | | Chemical | |
|---|---|---|---|---|
| | shift | Percentage | shift | Percentage |
| Sugar-5,6F₂BIM | (in D₂O)^{b} | (integration) | (in CD₃OD)^{b} | (integration) |
| Fucose | −138.52 | 42 (0.95) | −141.20 | 40 (—) |
| Galactose | −139.40 | 44 (1.00) | −139.10 | 40 (—) |
| Glc-NHAc | −138.55 | 14 (0.28) | −141.20 | 20 (—) |

^{a}470 MHz ¹⁹F NMR was used.
^{b}Trifluorotoulene was used as an internal standard at −63.72 ppm.

The results of the composition analysis of Lewis^{Y} (Fucα1-2Galβ1-4(fucα1-3)GlcNAcβ1-3Gal) by HPLC using Sugar-5,6F₂BIMs labeled: ¹⁹F are given in Table 29 below.

TABLE 29
Composition of Lewis^{Y} (Fucα1-2Galβ1-
4(fucα1-3)GlcNAcβ1-3Gal) by HPLC

| Sugar-5,6F₂BIM | Retention time (min) | Percentage |
|---|---|---|
| Fucose | 19.8 | 40 |
| Galactose | 17.5 | 40 |
| Glc-NHAc | 21.3 | 20 |

### Example 19

### Quantitation a Saccharide in a Liquid Sample

19.1 Materials and Methods

1. General

All chemicals and solvents were of analytical grade and used without further purification. Sugars (glucose, galactose, mannose, maltose, lactose, fructose and sucrose), iodine, acetic acid (AcOH), 2,3-naphthalenediamine and deuterium solvents were purchased from Merck & Co., Inc. (Darmstadt, Germany). Beverages were purchased from tea drinking store and the crop samples, e.g. soybean (non-GMO), rice (common rice) and wheat (low gluten flour), were purchased from traditional local market in Taipei city. NAIM labeling kit used in this study was provided by Sugarlighter Co., Inc. (New Taipei City, Taiwan).

2. General Procedure for Preparation of Sugar-Naim Derivatives

According to the previously reported procedures (Lin, C. et al., J. Org. Chem. 2008, 73, 3848-3853), glucose (2.0 mg, 11 μmol), 2,3-naphthalenediamine (2.0 mg, 13 μmol), and iodine (2.0 mg, 8 μmol) in AcOH (1.0 mL) was stirred at room temperature. The reaction completed in 3 hours as indicated by the TLC analysis. The mixture was concentrated under reduced pressure to give the sample of Glc-NAIM derivative, which was directly subjected to NMR measurement without further purification. This reaction protocol is applicable to prepare other sugar-NAIM derivatives, including those of mixed sugars, in smaller quantities.

Alternatively, mono- and disaccharides were converted to the sugar-NAIM samples by using an NAIM labeling kit that consists of three vials (Sugarlighter Co., New Taipei City, Taiwan). In brief, vial A containing 2,3-naphthalenediamine and vial B containing iodine in AcOH solution are used for conversion of saccharides to the NAIM derivatives. Vial C containing D₂O (1.0 mL) and a small amount of dimethylsulfoxide (DMSO) as internal standard is used in recording ¹H-NMR spectra.

3. General Procedure for Analysis of Common Sugars in Beverage

Beverage (50 μL) was taken and directly treated with an NAIM labeling kit. Pretreatment or dilution of the beverage sample is not required in this typical analysis. The sugar components in beverage were converted to the corresponding NAIM derivatives at room temperature for 3 hours using the reagents from vials A and B of the NAIM labeling kit. The resulting solution was concentrated under reduced pressure, and the residue was dissolved in vial C for ¹H-NMR measurement.

4. General Procedure for Analysis of the Monosaccharides Released from the Glycan of Food Crops

In a typical procedure, food crop (1.0 g) was ground for homogenization and washed with water (10 mL×2) to remove free monosaccharides. The dried material (1.0 mg) was treated with trifluoroacetic acid (TFA) (1 mL of 4 M aqueous solution) at 110° C. for 4 hours. The resulting aqueous solution was concentrated by rotary evaporation under reduced pressure at room temperature. The residue containing the released monosaccharide components was subsequently converted to the corresponding NAIM derivatives using NAIM labeling kit at room temperature for 3 hours. The sample was dissolved in deuterium oxide (D₂O) solution containing DMSO as internal standard for the ¹H-NMR measurement.

5. ¹H-NMR Analysis

The ¹H-NMR spectra were recorded on a Bruker AV600 MHz NMR spectrometer (Bruker BioSpin GmbH, Rheinstetten, Germany) with a 5 mm dual cryoprobe DCI ¹H/¹³C. The sugar-NAIM product was dissolved in D₂O (1.0 mL) containing DMSO as internal standard. Quantification of sugars was based on the integral areas of the characteristic proton signals (e.g. H-2 in Glc-NAIM) by comparison with that of DMSO (six protons of the two methyl groups at δ 2.73).

6. HPAEC-PAD Analysis

Beverage was 100-fold diluted in double-distilled water (dd-H₂O), and 10 μL of the sample was injected for HPAEC-PAD analysis. Alternatively, food crop (1 mg) was hydrolyzed and concentrated. The residue was 100-fold diluted in dd-H₂O, and 10 μL of the sample was injected for HPAEC-PAD analysis. The above-prepared carbohydrate samples were analyzed using a Dionex™ ICS-3000 DC equipment containing a gradient pump and an eluent degas module. Separation of carbohydrate molecules was carried out on a CarboPac PA-10 anion-exchange column (250×2 mm). The mobile phase contained 100 mM NaOH (eluent A) and 500 mM NaOAc (eluent B) in gradients. Eluent A was constant (100%) during 0-10 min, and gradient (100% to 0%) was produced during 10-30 min with eluent B. The flow rate was 0.25 mL min⁻¹. Carbohydrates were detected by pulsed amperometric detection (PAD) with a gold working electrode and a hydrogen reference electrode. The temperature was set at 25° C. and all analyses were carried out in duplicate.

19.2 Results

1. Derivatization and NMR Spectrometric Analysis of Aldo-Sugars

An aldose molecule inherently exists in solution as a mixture of the α and β anomeric isomers to display a rather complicate ¹H-NMR spectrum. By transformation of both aldose anomers to a single NAIM compound would simplify the ¹H-NMR analysis. An aldose (2 mg) was generally converted to the NA1M derivative at room temperature in 3 h by using an NAIM labeling kit that contains the reagents of 2,3-naphthlenediamine and iodine in acetic acid. After removal of acetic acid under reduced pressure, the residue of sugar-NAIM derivative without further purification was dissolved in D₂O for recording the ¹H-NMR spectrum. Instead of using the conventional but less accessible reagent (CH₃)₃SiCD₂CD₂CO₂Na, the readily available and cost-effective reagent DMSO was applied as internal standard, showing the two methyl groups as a singlet at δ 7.23. The NAIM derivatives of several mono- and disaccharides, including glucose (Glc), galactose (Gal), mannose (Man), rhamnose (Rha), arabinose (Ara), glucuronic acid (GlcUA), N-acetylglucose (GlcNAc), maltose (Mal) and lactose (Lac), were individually prepared and subjected to ¹H-NMR spectral analyses. Table 30 below lists the characteristic proton signals of these NAIM compounds.

TABLE 30
¹H-NMR data (600 MHz, D₂O) of the NAIM derivatives
prepared from common mono- and disaccharides

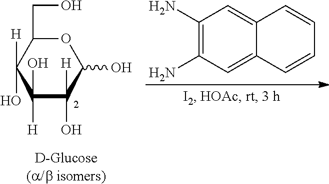

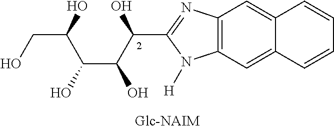 [SMILES: OC[C@@H](O)[C@@H](O)[C@H](O)[C@@H](O)c1nc2cc3ccccc3cc2[nH]1]

| | Chemical shift (δ, ppm)^{a} | | | | |
|---|---|---|---|---|---|
| Compound | H-2 | H-3 | H-4 | H-5 | H-6 |
| Glc-NAIM^{b} | 5.38 | 4.40 | 3.85 | 3.77 | 3.75, 3.62 |
| Gal-NAIM^{b} | 5.54 | 4.15 | 4.06 | 3.95 | 3.80, 3.80 |
| Man-NAIM | 5.59 | 4.69 | 4.28 | 3.93 | 3.87, 3.73 |
| Rha-NAIM | 5.13 | 4.33 | 3.95 | 3.72 | — |
| Ara-NAIM | 5.49 | 4.11 | 3.97 | 3.96, 3.80 | — |
| GlcUA-NAIM | 5.53 | 4.41 | 4.13 | 4.31 | — |
| GlcNAc-NAIM | 5.44 | 4.51 | 3.90 | 3.84 | 3.73, 3.60 |
| Mal-NAIM^{b} | 5.52 | 3.89 | 3.66 | 4.11 | 3.93, 3.82 |
| Lac-NAIM^{b} | 5.56 | 4.43 | 4.14 | 4.08 | 3.93, 3.84 |

^{a}The signal of HDO is set at δ 4.80.
^{b}DMSO (0.1%, v/v) was also included as internal standard at δ 2.73.

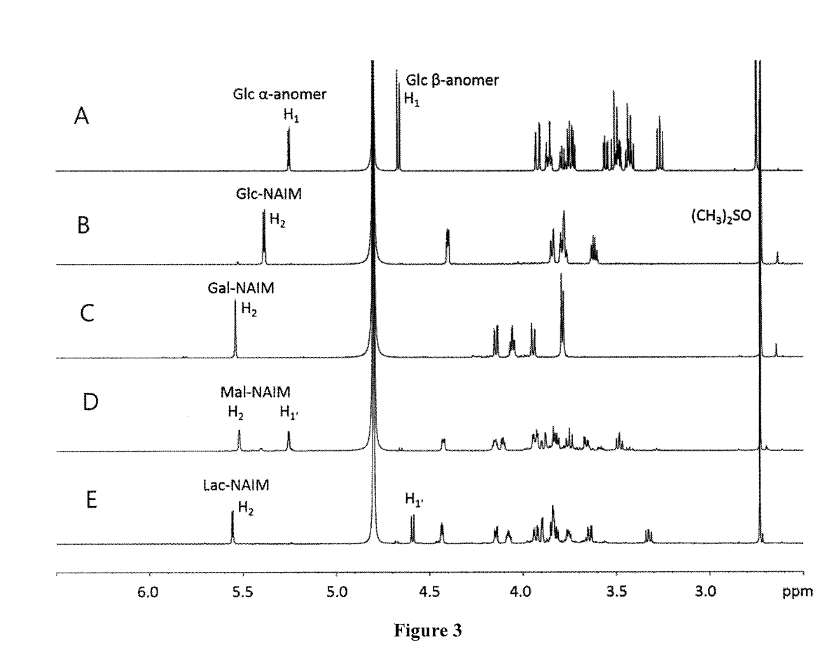

Glucose exists as a mixture of α and β anomers, which showed the C-1 protons at δ 5.24 and 4.66, respectively (FIG. 3, (A)). In comparison, the ¹H-NMR spectrum of Glc-NAIM was much simplified, and the 11-2 shifted downfield to δ 5.38 as a doublet (J=5.4 Hz, FIG. 3, (B)). The characteristic H-2 of Gal-NAIM appeared at δ 5.54 (d, J=1.8 Hz, FIG. 3, (C)). The disaccharide derivative Mal-NAIM exhibited H-2 at δ 5.52 (d, J=1.8 Hz) and the glycosidic proton (H-1′) at δ 5.25 (d, J=3.6 Hz, FIG. 3, (D)), whereas Lac-NAIM displayed H-2 at δ 5.56 (d, J=4.2 Hz) and H-1′ at δ 4.59 (d, J=7.8 Hz, FIG. 3, (E)). These sugar-NAIM derivatives consistently showed the characteristic patterns of C-2 protons in the region of 5.1-5.6 ppm along with other well recognizable proton signals in the ¹H-NMR spectra. Thus, the parental sugars could be easily inferred from their corresponding NAIM derivatives using ¹H-NMR spectrometry. Specifically, this ¹H-NMR method is versatile to distinguish glucose from mannose (C2-epimer) and galactose (C4-epimer). Maltose and lactose were also readily differentiated by the ¹H-NMR spectra of their NAIM derivatives.

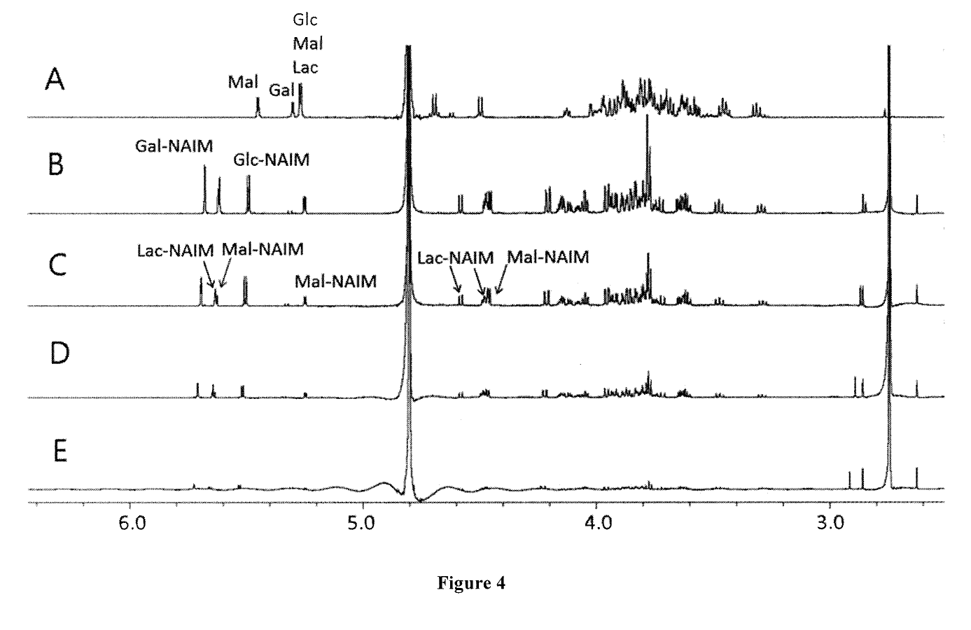

To demonstrate the advantage of using NAIM derivatives in ¹H-NMR analysis of sugar mixture, a sample containing 4 aldoses (Glc, Gal, Mal and Lac) in equal amounts (5 mg each) was subjected to NAIM derivatization using an NAIM labeling kit, followed by quantitative analysis using ¹H-NMR spectrometry. The parental sugars Glc, Mal and Lac could not be easily quantified because their C-1 protons overlapped on the same position (FIG. 4, (A)). In contrast, the NAIM derivatives were easily distinguished by their C-2 protons with the diagnostic patterns at distinct chemical shifts, which might have small variation due to intermolecular interactions (FIGS. 4, (B) (E)). Taking the integration area of the peak at δ 2.73 for the two methyl groups of DMSO (6 protons) as reference, one could calculate the amount of each NAIM derivative from its H-2 signal. The glycoside protons (H-1′) of Mal-NAIM (at δ 5.25) and Lac-NAIM (at δ 4.59) could also be utilized for the quantitative analysis. The sugar-NAIM mixture in a small amount (as low as 0.5 mg of each component) could be detected with S/N≧5 by ¹H-NMR spectroscopy (FIG. 4, (D)).

2. NMR Spectrometric Analysis of Six Common Sugars

Since Jul. 1 of 2014, all beverages on Taiwan market must be labeled in nutrition facts with the total amount of six common sugars (Glc, Gal, Fru, Mal, Lac and Suc), even the recommended content for each sugar is not yet given by TFDA. We first examined the ¹H-NMR spectrum of a mixture containing these six common sugars. In this spectrum, fructose has no diagnostic peak for identification. Furthermore, maltose could not be quantified because its anomeric protons (H-1α and H-1β) overlapped with those of glucose, and its glycosidic proton (H-1′β) was not well separated from that of sucrose (FIG. 5, (A)). Alternatively, the aldose components including Glc, Gal, Mal and Lac in the sample were converted to the corresponding NAIM derivatives on treatment with an NAIM labeling kit. The NAIM derivatives were readily distinguished by their characteristic signals in the ¹H-NMR spectrum (FIG. 5, (B)). Taking the integration areas of the characteristic proton signals, one can calculate the amount of each NAIM derivative, for example, from the H-2 signals of Glc-NAIM at δ 5.47, Gal-NAIM at δ 5.65, Mal-NAIM at δ 5.57 and Lac-NAIM at δ 5.59.

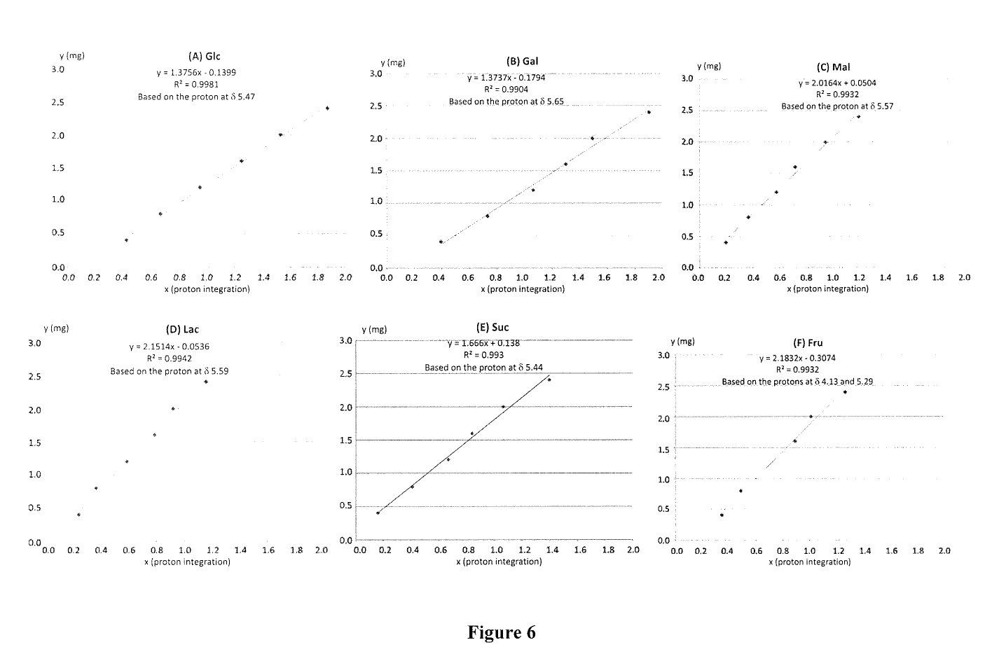

Furthermore, we established the calibration lines for individual sugar component based on their characteristic proton signals in the ¹H-NMR spectrum using DMSO at 0.03% concentration (4.3 μmol) as internal standard (FIG. 6, (A)-(F)). For example, the quantity of glucose (y) is calculated from the relative integration (x) of the selected proton signal of Glc-NAIM at δ 5.47 (FIG. 6, (A)): y=1.3756x-0.1399 with a high coefficient of determination (R²>0.99).

Sucrose, a nonreducing sugar, was retained without oxidative condensation by 2,3-naphthalenediamine under such reaction conditions. Nonetheless, sucrose can be identified by its glycosidic proton (H-1) at δ 5.44 and another proton at δ 5.23. Accordingly, the calibration line is established for quantification of sucrose (FIG. 6, (E)): y=1.666x+0.138 using the selected proton signal at δ 5.44.

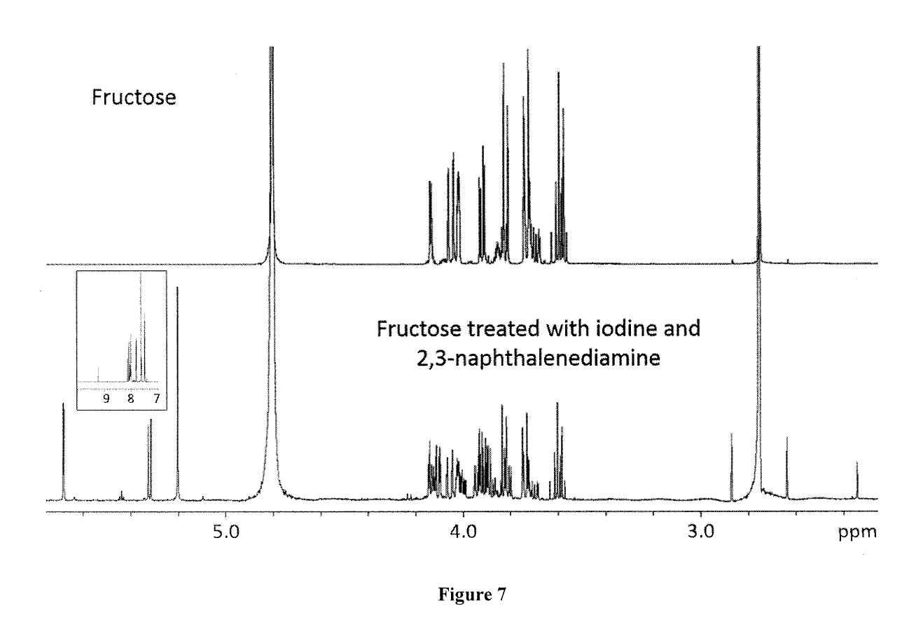

Interestingly, some small but distinct peaks were also observed at δ 5.20 (s), 5.32 (d, J=7.2 Hz), 5.38 (t, J=4.8 Hz), 5.66 (d, J=1.8 Hz) and 9.24 (s) in FIG. 5, (B). Though fructose could not form a NAIM derivative, we surmised that these peaks might belong to the intermediates due to the reaction of fructose with 2,3-naphthalenediamine. We thus performed a separate experiment by treating fructose with 2,3-naphthalenediamine in D₂O solution (without addition of iodine), and recorded the ¹H-NMR spectrum (FIG. 7, (B)). In comparison with the spectrum of fructose (FIG. 7, (A)), the emerging proton signals occurring in FIG. 5, (B) also appeared in FIG. 7, (B). The signals at δ 5.20 (s), 5.32 (d), and 5.66 (d) were tentatively ascribed to the structure of fructose-enamine [A] (containing the E and Z isomers), while the smaller signals at δ 5.38 (t, J=4.8 Hz) and 9.24 (s) might be attributable to the a-amino aldehyde [B] as a tautomer of [A]. Taking the combined integration (x) of the signals at δ 4.13 (d) and 5.29 (s) for unchanged fructose and the enamine [A] derivative, the original content of fructose (y) could be estimated by the following equation (FIG. 6, (F)): y=2.18x−0.31.

3. NMR Spectrometric Analysis of Six Common Sugars in Beverage

We have previously applied HPLC method to determine the contents of carbohydrates in five types of beverages, including fruit juice, yogurt, coffee drink, milk tea and flavored milk in Taiwan (Hung, W.-T.et al., Func. Food Health Dis. 2016, 6, 234-245). Here, we applied ¹H-NMR spectrometric method to determine the composition of six common sugars in beverages.

The samples of milk tea and soymilk were purchased from shops and street vendors, respectively. The sugar contents of these samples are marked as H (high), M (medium), L (low) and F (free) according to their sugar contents. Individual beverage sample (50 μL) was taken and directly treated with an NAIM labeling kit for 3 hours at room temperature. After removal of acetic acid under reduced pressure at room temperature, the residue of sugar-NAIM derivatives, without further purification, was dissolved in D₂O (1.0 mL) containing 0.1% DMSO as internal standard for the ¹H-NMR analysis. Table 31 below shows the quantities of individual sugars in each sample. In all the test samples of milk tea and soymilk, no glucose or fructose was found. However, in 100 mL of any kind of milk tea samples were found 1.0 g of lactose and 0.1 g of galactose due to the added milk. In contrast, there was no lactose or galactose found in the soymilk samples. Maltose was detected in all the soymilk samples which may come from fermentation of soybean starch. Sucrose appeared to be the sole sugar that was added by vendors to milk tea and soymilk. The content of sucrose was as high as 18.4 g in 100 mL of milk tea (H) compared to no sucrose in the original milk tea (F). It was noted that even the so call sugarless soymilk (F) still contained an appreciable amount of sucrose (1.8 g) that might be attributable to the original sugar of soybean. Therefore, by drinking a cup (300 mL) of so call low-sugar-content milk tea or medium-sugar-content soymilk, one may still intake excessive sugar, predominating in sucrose, over the daily need (25 g) as that is recommended by WHO and nutritionists.

TABLE 31
Quantitative analysis of common sugars in beverages using ¹H-
NMR spectrometry (600 MHz, D₂O containing 0.1% DMSO).^{a}

| Sample | Glc | Gal | Fru | Mal | Lac | Suc | Total sugar |
|---|---|---|---|---|---|---|---|
| (100 mL)^{a} | (g) | (g) | (g) | (g) | (g) | (g) | (g) |
| Milk tea (H) | 0 | 0.1 | 0 | 0 | 1.0 | 18.4 | 19.5 |
| Milk tea (M) | 0 | 0.1 | 0 | 0 | 1.0 | 13.5 | 14.6 |
| Milk tea (L) | 0 | 0.1 | 0 | 0 | 1.0 | 10.9 | 12.0 |
| Milk tea (F) | 0 | 0.1 | 0 | 0 | 1.0 | 0 | 2.1 |
| Soymilk (H) | 0 | 0 | 0 | 0.1 | 0 | 12.6 | 13.5 |
| Soymilk (M) | 0 | 0 | 0 | 0.1 | 0 | 9.3 | 9.4 |
| Soymilk (F) | 0 | 0 | 0 | 1.0 | 0 | 1.8 | 2.8 |

^{a}The sample is subjected to NAIM labeling, and the amounts of saccharides are deduced from their corresponding NAIM derivatives. The sugar contents in the samples of milk tea and soymilk are denoted as H (high), M (medium), L (low) and F (free) in parentheses, respectively.

In comparison, we also performed high-performance anion-exchange chromatography with pulsed amperometric detection (HPAEC-PAD) of the sugar contents in beverage, and found that the results were consistent with the data of ¹H NMR analysis shown in Table 31. HPAEC-PAC method is a very sensitive method in carbohydrate analysis; however, decay of electrodes may cause problems in calibration and quantitative measurement. It was noted that HPAEC-PAC is usually operated with minute amount (10⁻¹² to 10⁻¹⁵ mol) of beverage sample, even a small experimental error may be amplified to a large deviation in backward counting the real sugar content. These problems are less obvious by using ¹H-NMR spectrometry that is usually operated with 10⁻⁵ mol of carbohydrate sample.

4. NMR Spectrometric Analysis of Sugar Composition in Food Crops

We also investigated the feasibility of using ¹H-NMR spectrometric method to quantify the monosaccharides released from the glycans in food crops. A food crop (1 mg) was hydrolyzed in 4 M trifluoroacetic acid at 110° C. for 4 h. The crude hydrolysate was treated with an NAIM labeling kit at room temperature for 3 hours to give a sample containing the corresponding monosaccharide-NAIM derivatives. The sample was dissolved in D₂O solution containing 0.1% (v/v) of DMSO as internal standard for the ¹H-NMR measurement. As shown in Table 32, starch is the main ingredient in all the tested food crops, yielding 1.4-11.2% (w/w) glucose after hydrolysis. Galactose was also found in 1.0% and 0.3% in soybean and potato, respectively. Although soybean has the lowest sugar content among the test food crops, it contains an appreciable amount of arabinose (3.7%). In the crop hydrolysate, there are also other types of monosaccharides and oligosaccharides, such as maltose and its oligomers, presumably due to incomplete hydrolysis. However, no lactose or sucrose was found in all the crop samples. In this study, only a small amount (1 mg) of the raw material is required for the NAIM derivatization and ¹H-NMR analysis to quantify the monosaccharide contents. This method is potentially utilized in profiling and fingerprinting of food crops.

TABLE 32
Quantitative analysis of the glycan composition in food crops using
¹H-NMR spectrometry (600 MHz, D₂O containing 0.1% DMSO).^{a}

| | | | | | Total sugar |
|---|---|---|---|---|---|
| Crop (1 mg)^{a} | Glc (μg) | Gal (μg) | Ara (μg) | Others (μg) | (μg) |
| Rice | 112.2 | N.D.^{b} | N.D.^{b} | N.D.^{b} | 112.2 |
| Soybean | 14.0 | 10.3 | 37.3 | 11.3 | 72.9 |
| Wheat | 102.7 | N.D.^{b} | 9.5 | 13.4 | 125.6 |
| Potato | 82.9 | 2.8 | 4.4 | N.D.^{b} | 90.1 |
| Corn | 89.1 | N.D.^{b} | 2.0 | 2.2 | 93.3 |
| Mung bean | 91.2 | N.D.^{b} | N.D.^{b} | N.D.^{b} | 91.2 |
| Yam bean | 77.0 | 0.2 | 8.2 | 2.5 | 88.0 |
| Taro | 93.7 | N.D.^{b} | 3.4 | N.D.^{b} | 97.0 |
| Banana | 65.3 | N.D.^{b} | 4.7 | N.D.^{b} | 70.0 |

^{a}The sample is subjected to acidic hydrolysis, followed by NAIM labeling.
^{b}N.D. is not detected.

19.3 Conclusion

We demonstrate in this study that NMR spectrometry can be effectively utilized to quantify the common sugar ingredients in beverage and food crops via a simple treatment with an NAIM labeling kit. The NAIM reaction is smoothly performed at room temperature, and the product without further purification is directly subjected to the ¹H-NMR analysis. This operation renders the anomeric isomers of an aldose to a single NAIM derivative that shows the characteristic H-2 signal at downfield for diagnosis and quantitative analysis by ¹H-NMR spectrometry. Sucrose is unchanged under such NAIM reaction conditions, and is readily identified by its glycosidic proton (H-1) at δ 5.44. The content of fructose ingredient can be estimated from the calibration line that is established by taking the combined integration of the proton signals at δ 4.13 (d) and 5.29 (s) for unchanged fructose and the enamine derivative [A]. Thus, even small amounts of sugar ingredients in 50 μL of beverage and 1 mg of food crop can be quantified by the method using NAIM derivatization for ¹H-NMR analysis. This method is potentially useful for profiling and fingerprinting of food crops.

While the invention has been described with reference to the preferred embodiments above, it should be recognized that the preferred embodiments are given for the purpose of illustration only and are not intended to limit the scope of the present invention and that various modifications and changes, which will be apparent to those skilled in the relevant art, may be made without departing from the spirit and scope of the invention.

I/we claim:

1. A method for quantitating a saccharide in a liquid sample, comprising incubating the liquid sample with 2,3-naphthalenediamine in the presence of iodine to allow a naphthimidazole group to be linked to the saccharide to obtain a first mixture; obtaining an ¹H-NMR spectrum of the first mixture; and comparing, in said ¹H-NMR spectrum, the intensity or integral of a first proton signal corresponding to the saccharide to the intensity or integral of a second proton signal corresponding to an internal standard present in the first mixture.

2. The method of claim 1, wherein the internal standard is DMSO, tetramethylsilane, or (CH₃)₃SiCO₂Na.

3. The method of claim 1, wherein the liquid sample is prepared by acid hydrolysis of a solid sample.

4. The method of claim 1, wherein the first proton signal is a characterizing proton signal of the saccharide, and the second proton signal is a characterizing proton signal of the internal standard.

5. The method of claim 1, wherein the saccharide is selected from the group consisting of glucose (Glc), galactose (Gal), fructose (Fru), lactose (Lac), maltose (Mal), sucrose (Suc), mannose (Man), rhamnose (Rha), arabinose (Ara), glucuronic acid (GlcUA), and N-acetylglucose (GlcNAc).

6. The method of claim 4, wherein the first proton signal is a vinyl proton signal.

7. The method of claim 4, wherein the internal standard is DMSO.

8. The method of claim 7, wherein the second proton signal includes the NMR signals of six protons of the two methyl groups of DMSO at δ 2.73.
